(19) United States
(12) Patent Application Publication
(10) Pub. No.: US 2024/0336567 A1
(43) Pub. Date: Oct. 10, 2024

(54) PESTICIDAL AND HERBICIDAL COMPOUNDS AND METHODS OF USE THEREOF

(71) Applicant: AG Plenus Ltd, Rehovot (IL)

(72) Inventors: Boaz INBAL, Kfar Shmu'el (IL); Ezequiel WEXSELBLATT, Shoeva (IL); Yehoshua FREUD, Maale Adumim (IL); Yoav ATSMON-RAZ, Rehovot (IL); Willms LOTHAR, Hillscheid (DE)

(73) Assignee: AG Plenus Ltd, Rehovot (IL)

(21) Appl. No.: 18/272,577

(22) PCT Filed: Jan. 23, 2022
(86) PCT No.: PCT/IL2022/050095

§ 371 Date: Jul. 16, 2023

(30) Foreign Application Priority Data
Jan. 25, 2021 (IL) 280384

(51) Int. Cl. C07D 213/79; A01N 37/30; A01N 37/40; A01N 43/08; A01N 43/10; A01N 43/36; A01N 43/40; A01N 43/54; A01N 43/56; A01N 43/653; A01N 43/76; A01N 43/78; A01N 43/80; A01N 43/82; A01P 13/00; C07C 217/56; C07C 233/83; C07D 207/36; C07D 207/38; C07D 231/20; C07D 239/34; C07D 249/12; C07D 261/18; C07D 263/38; C07D 271/113; C07D 277/56; C07D 307/68; C07D 333/40

(52) U.S. Cl. CPC C07D 213/79; A01N 37/30; A01N 37/40; A01N 43/08; A01N 43/10; A01N 43/36; A01N 43/40; A01N 43/54; A01N 43/56; A01N 43/653; A01N 43/76; A01N 43/78; A01N 43/80; A01N 43/82; A01P 13/00; C07C 217/56; C07C 233/83; C07D 207/36; C07D 207/38; C07D 231/20; C07D 239/34; C07D 249/12; C07D 261/18; C07D 263/38; C07D 271/113; C07D 277/56; C07D 307/68; C07D 333/40

(57) ABSTRACT

The present invention relates to novel pesticidally and/or herbicidally active compounds, agrochemical composition thereof, methods of preparation thereof, and uses thereof for controlling the growth of undesirable plants (e.g., weeds), for example in crop fields.

### FIELD OF THE INVENTION

The present invention relates to novel pesticidally and/or herbicidally active compounds, agrochemical composition thereof, methods of preparation thereof, and uses thereof for controlling the growth of undesirable plants (e.g., weeds), for example in crop fields.

### BACKGROUND OF THE INVENTION

Weeds often interfere with efficient utilization of land and water resources and typically compete with desired plants for water, nutrients, light, carbon dioxide, and space. Many weeds are also aesthetically displeasing, especially when the weeds appear within a stand of a desired plant, such as St. Augustine grass or Kentucky bluegrass in a homeowner's lawn. Weeds may also obstruct visibility, become fire hazards around buildings, and reduce the efficiency of irrigation systems. When weeds appear in watercourses, such as rivers and lakes, the weeds may contribute to poor water quality, making the water unsuitable for culinary and industrial uses. Furthermore, some weeds act in a poisonous fashion against other plants, animals, and humans by secreting toxic substances known as alleopathic compounds or by spreading agents that may cause allergies and/or disease. Finally, weeds provide shelter for insects and rodents that spread disease or are otherwise harmful to desired plants, animals, or humans.

Weeds cause agricultural losses to crops that consistently exceed losses caused by other classes of agricultural pests, year after year. Besides reducing the quality of the crop, weed infestation may reduce achievable crop yield by up to 100% of the theoretically achievable yield. A number of approaches, including mechanical, agricultural, biological, and chemical techniques, have evolved in an attempt to control weed infestation.

Mechanical means, such as hand pulling, hoeing or cultivation, deep plowing, clipping, mowing, burning and/or mulching, may be employed in an attempt to eradicate or control weeds. Also, cover crops may be planted to keep the ground covered when not growing more valuable crops and thus weed infestation that would ordinarily be expected to occur in bare ground areas is typically minimized. Crop rotation and planting of “smother” crops that are adapted to grow more vigorously than weeds have also been attempted as means of controlling weed infestations. Besides these mechanical and agricultural techniques, biological methods of weed control, such as introduction of predator populations that feed on the weeds and thereby reduce weed population, have also been attempted.

Mechanical, agricultural, and biological methods of weed control, while sometimes helping to reduce the extent of weed infestations, are not fully satisfactory. First, mechanical and agricultural techniques are quite labor intensive and require use of limited physical and capital resources. Furthermore, environmental factors beyond the control of the farmer or homeowner, such as excessive rainfall, may diminish the effectiveness of these mechanical and agricultural techniques. Likewise, biological techniques, such as introduction of predator populations, are not entirely satisfactory, since the predators may not be selective for only the weed population.

Chemically active herbicides represent another potential weed control technique. These chemical herbicides may be broken down into pre-emergent herbicides and post-emergent herbicides. Pre-emergent herbicides typically interfere with germination of weed seeds, whereas post-emergent herbicides kill the weeds after the weed seeds have germinated and weed growth has begun.

Pre-emergent herbicides may be effective when present at the required dosage at the time weed seed germination is ready to occur. However, this timing issue points out a major problem with respect to pre-emergent herbicides. Specifically, if the pre-emergent herbicide is not applied, or degrades, prior to weed seed germination, the weed seeds are free to germinate and begin growing into mature weeds. Additionally, pre-emergent herbicides are typically weed specific and are not equally effective against all types of weeds. The timing problem present with pre-emergent herbicides may be avoided by employing post-emergent herbicides and by applying the post-emergent herbicide only after the weed seeds have germinated and the weeds are actively growing. However, many presently available post-emergent herbicides are non-selective herbicides and therefore will kill desirable plants in addition to weeds.

Many pre- and post-emergent herbicides also suffer from another problem. Specifically, many pre-emergent herbicides and post-emergent herbicides are either moderately or highly toxic to humans and animals, and may thereby have damaging effects far beyond the intended weed control effect. Toxic herbicides may cause injury either immediately or over the long term to persons applying the herbicides and to persons present when the herbicides are applied. Also, residual concentrations of toxic herbicides that remain in the soil or water after application of the herbicide may pose a significant threat to human beings and to animals, including land-based animals and amphibians and fish, upon contact with the treated area or runoff from the treated area. Furthermore, public alarm about the use of toxic chemicals as herbicides and their potential widespread and long-term effects on environmental quality dictate against the continued use of these toxic herbicides.

There is a need for a pesticidal and/or herbicidal solution that avoids the critical timing issues of pre-emergent herbicide applications. Furthermore, there is a need for a pesticidal and/or herbicidal solution that avoids the toxic effects of presently available pre-emergent and post-emergent herbicides on human beings, animals and the environment generally. Furthermore, there is a need for an economically efficient post-emergent weed technique that selectively controls weeds without destroying or hindering the growth of desired plants. In addition, there is a need for a composition that reduces the amount of herbicides necessary to obtain sufficient weed control while minimizing the harm to crop plants.

As more weeds become resistant to herbicides, alternative compositions with high weed control are desired. Further, as no-till farming continues to increase in popularity, there is a greater need for effective herbicides. A composition with effective weed control and lower dosage rate will lead to increased crop plant yields, and decreased environmental, human, and mammalian health concerns.

### SUMMARY OF THE INVENTION

In various embodiments, this invention is directed to a compound represented by the structure of formula I(d), I(e), or any one of the compounds as defined in Table 2 hereinbelow, or its agrochemically acceptable salt, zwitterion (inner salt), stereoisomer, tautomer, hydrate, N-oxide, reverse amide analog, isotopic variant (e.g., deuterated analog), or any combination thereof.

In various embodiments, this invention is directed to a pesticidal or an herbicidal compound represented by the structure of formula I-I(e), as defined herein below or its agrochemically acceptable salt, zwitterion (inner salt), stereoisomer, tautomer, hydrate, N-oxide, reverse amide analog, isotopic variant (e.g., deuterated analog), or any combination thereof.

In various embodiments, this invention is directed to a method for controlling the growth of undesired plants, comprising applying a compound represented by the structure of formula I-I(e), or its agrochemically acceptable salt, zwitterion (inner salt), stereoisomer, tautomer, hydrate, N-oxide, reverse amide analog, isotopic variant (e.g., deuterated analog), as defined herein below, or an agrochemical composition thereof, to a crop field.

In some embodiments, this invention is directed to a compound represented by any one of the compounds presented in Tables 1 and/or 2 herein below, or its agrochemically acceptable salt, zwitterion (inner salt), stereoisomer, tautomer, hydrate, N-oxide, reverse amide analog, isotopic variant, or any combination thereof. In various embodiments, the compound is not: 6-(2-aminopropoxy)picolinic acid; 6-(2-aminopropoxy)pyrazine-2-carboxylic acid, 2-(2-aminopropoxy)pyrimidine-4-carboxylic acid; 6-(2-aminopropoxy)-3-fluoropicolinic acid; 6-(2-aminopropoxy)-4-fluoropicolinic acid; 6-(2-aminobutoxy)picolinic acid; 6-((2-aminocyclopentyl)oxy)picolinic acid; methyl 6-(2-aminopropoxy)picolinate; or ethyl 6-(2-aminopropoxy)picolinate.

In various embodiments, the compound is a substantially pure single stereoisomer. In various embodiments, the compound is a substantially pure single enantiomer. In various embodiments, the compound is a mixture of stereoisomers. In various embodiments, the substantially pure stereoisomer or enantiomer has a purity higher than 90%, preferably higher than 95%, most preferably higher than 98%. In various embodiments, the compound is an herbicide or a pesticide (i.e., herbicidal or pesticidal compound).

In various embodiments, this invention is directed to an agrochemical composition comprising a pesticidally and/or herbicidally effective amount of a compound according to this invention, and an agrochemically-acceptable diluent or carrier. In various embodiments, the compound is for use in controlling the growth of undesired plants. In various embodiments, the compound is a substantially pure single stereoisomer, a mixture of stereoisomers, or a racemate. In various embodiments, the stereoisomer is an enantiomer. In various embodiments, the substantial pure stereoisomer has a purity higher than 90%, preferably higher than 95%, most preferably higher than 98%.

In various embodiments, this invention is directed to a method of controlling the growth of undesired plants, comprising applying a compound according to this invention or an agrochemical composition according to this invention, to crop fields.

In various embodiments, this invention is directed to a compound according to this invention, or an agrochemical composition according to this invention, for use in controlling the growth of undesired plants. In some embodiments, the plant is a eudicot (dicot) or a monocotyledon (monocot). In some embodiments, the plant is a weed. In some embodiments, the weed comprises: Abutilon theophrasti, Amaranthus palmeri, Ambrosia artemisiifolia, Alopecurus myosuroides, Avena sterilis, Chenopodium album, Conyza Canadensis, Digitaria sanguinalis, Echinochloa colona, Euphorbia heterophylla, Lolium perenne, Lolium rigidum, Matricaria chamomilla, Phalaris paradoxa, Poa annua, Portulaca oleracea, Setaria viridis, Solanum nigrum or any combination thereof. In some embodiments, the dicot plant is Arabidopsis thaliana, and/or the monocot plant is Dactyloctenium aegyptium or Eragrostis teff. In some embodiments, the compound is for use in pre-plant treatments, pre-emergence treatments, post-emergence treatments, or any combination thereof; each represents a separate embodiment according to this invention.

### BRIEF DESCRIPTION OF THE DRAWINGS

FIG. 1 depicts the NMR spectrum of compound 116.

FIG. 2 depicts the NMR spectrum of compound 106.

### DETAILED DESCRIPTION OF THE INVENTION

In various embodiments, this invention is directed to a compound represented by the structure of formula (I):

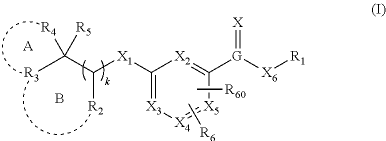

wherein
A and B rings are absent, or are each independently a substituted or unsubstituted aromatic or heteroaromatic ring system (e.g., B: aryl, pyridine), or a substituted or unsubstituted C₃-C₁₀ cycloalkyl (A: cyclopropyl, B: cyclopentyl, cyclohexyl), or a substituted or unsubstituted C₃-C₁₀ heterocyclic ring (e.g., pyrrolidine);
R₁ is H, substituted or unsubstituted C₁-C₁₀ linear or branched, or C₃-C₈ cyclic alkyl (e.g., methyl, ethyl, propyl, butyl, CH₂—CCH, CH₂—COOH, CH₂CH₂COOH), C(O)—R₁₀ (e.g., C(O)—CH₃), R₈—OH (e.g., CH₂—OH), R₈—COO—R₁₀ (e.g., CH₂—COOH, CH₂—COO—CH₃, CH₂—COO—CH₂CH₃, CH₂—CH₂—COOH, CH₂—CH₂—COO—CH₃, CH₂—CH₂—COO—CH₂CH₃), R₈—SH, —R₈—O—R₁₀, (e.g., —CH₂—O—CH₃), R₈—(C₃-C₈ cycloalkyl), R₈-(3-8 membered heterocyclic ring), CF₃, NH₂, NHR, N(R)₂, R₈—N(R₁₀)(R₁₁), C(O)H, C₁-C₈ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
R₂ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, R₈—N(R₁₀)(R₁₁), C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
R₃ is H, substituted or unsubstituted C₁-C₁₀ linear or branched, or C₃-C₈ cyclic alkyl (e.g., methyl, ethyl, propyl, CH₂—CCH, C(H)(OH)(CH₃)), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂); C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
or R₂ and R₃ are joined to form a C₃-C₈ substituted or unsubstituted, saturated or unsaturated, aliphatic (e.g. cyclopentyl, cyclohexyl), or aromatic (e.g. pyridine), carbocyclic or heterocyclic ring (e.g. pyrrolidine);
R₄ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂), C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
or R₃ and R₄ are joined to form a substituted or unsubstituted, saturated or unsaturated, aliphatic or aromatic, carbocyclic or heterocyclic 3-8 membered ring (e.g. cyclopropyl), C═O or ═N—OH;
R₅ is H, OH, SH, NH₂, NHNH₂, NHR, N(R₁₀)(R₁₁) (e.g., N(H)CH₃, N(H)CH₂CH₃), R₈—N(R₁₀)(R₁₁) (e.g. CH₂—NH₂); or R₂ and R₅ are joined to form a substituted or unsubstituted 4-8 membered heterocyclic ring (e.g. pyrrolidine);
R₆ and R₆₀ are each independently H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₈ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₈ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₈ linear or branched, substituted or unsubstituted alkenyl (e.g., ethenyl), C₂-C₈ linear or branched, substituted or unsubstituted alkynyl (e.g., ethynyl, CC—CH₂-cyclobutyl, CC—CF₃), C₁-C₈ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₈ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₈ linear or branched thioalkoxy, C₁-C₈ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl;
X₁ is O, S, NH, NR (e.g., N—CH₃), S═O, SO₂; or X₁ and R₄ are joined to form a 4-8 membered heterocyclic ring (e.g., pyrrolidine);
X₂, X₃, X₄, X₅ are each independently C or N;
X₆ is O, NH, NR or CH₂;
G=X is C═O or SO₂ or G(═X)—X₆—R₁ is a tetrazole moiety;
R₈ is [CH₂]p
   wherein p is between 1 and 10;
R₉ is [CH]_{q}, [C]_{q}
   wherein q is between 2 and 10;
R₁₀ and R₁₁ are each independently H, CN, C₁-C₈ linear or branched alkyl (e.g., methyl, ethyl), C(O)R (e.g., C(O)(OCH₃)), or S(O)₂R;
or R₁₀ and R₁₁ are joined to form a substituted or unsubstituted 3-8 membered heterocyclic ring,
R is H, C₁-C₈ linear or branched alkyl (e.g., methyl, ethyl), C₁-C₈ linear or branched alkoxy (e.g., methoxy), phenyl, aryl or heteroaryl, or two gem R substituents are joined together to form a 5 or 6 membered heterocyclic ring;
k is an integer number between 1 and 3 (e.g., 1, 2);
   or agrochemically acceptable salt, zwitterion (inner salt), stereoisomer, tautomer, hydrate, N-oxide, reverse amide analog, isotopic variant (e.g., deuterated analog), or any combination thereof.

In various embodiments, if k is not 1, then R₂ is H. In various embodiments, if k is 2, then R₂ is H. In various embodiments, if k is 3, then R₂ is H.

In various embodiments, the term “substituted” refers to substituted with at least one substitution selected from: F, Cl, Br, I, C₁-C₈ linear or branched alkyl, C₂-C₈ linear or branched alkenyl (e.g., ethenyl), C₂-C₈ linear or branched alkynyl, alkyl amide (i.e., C(O)NH—R or NHC(O)—R), alkyl ester (i.e., COOR or OC(O)—R), OH, alkoxy, N(R)₂, NH₂, CF₃, aryl, phenyl, heteroaryl, C₃-C₈ cycloalkyl, halophenyl, CN and NO₂.

In various embodiments, this invention is directed to a compound represented by the structure of formula I(a):

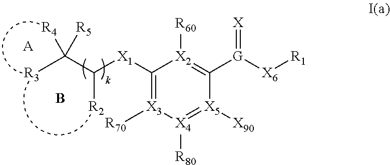

wherein
A and B rings are absent, or are each independently a substituted or unsubstituted aromatic or heteroaromatic ring system (e.g., B: aryl, pyridine), or a substituted or unsubstituted C₃-C₁₀ cycloalkyl (A: cyclopropyl, B: cyclopentyl, cyclohexyl), or a substituted or unsubstituted 3-10 membered heterocyclic ring (e.g., pyrrolidine);
R₁ is H, substituted or unsubstituted C₁-C₁₀ linear or branched, or C₃-C₈ cyclic alkyl (e.g., methyl, ethyl, propyl, butyl, CH₂—CCH, CH₂—COOH, CH₂CH₂COOH), C(O)—R₁₀ (e.g., C(O)—CH₃), R₈—OH (e.g., CH₂—OH), R₈—COO—R₁₀ (e.g., CH₂—COOH, CH₂—COO—CH₃, CH₂—COO—CH₂CH₃, CH₂—CH₂—COOH, CH₂—CH₂—COO—CH₃, CH₂—CH₂—COO—CH₂CH₃), R₈—SH, —R₈—O—R₁₀, (e.g., —CH₂—O—CH₃), R₈—(C₃-C₈ cycloalkyl), R₈-(3-8 membered heterocyclic ring), CF₃, NH₂, NHR, N(R)₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₂ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₃ is H, substituted or unsubstituted C₁-C₁₀ linear or branched, or C₃-C₈ cyclic alkyl (e.g., methyl, ethyl, propyl, CH₂—CCH, C(H)(OH)(CH₃)), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂); C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₂ and R₃ are joined to form a C₃-C₈ substituted or unsubstituted, saturated or unsaturated, aliphatic (e.g. cyclopentyl, cyclohexyl), or aromatic (e.g. pyridine), carbocyclic or heterocyclic ring (e.g. pyrrolidine);
R₄ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂), C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₃ and R₄ are joined to form a substituted or unsubstituted, saturated or unsaturated, aliphatic or aromatic, carbocyclic or heterocyclic 3-8 membered ring (e.g. cyclopropyl), C═O or ═N—OH;
R₅ is H, OH, SH, NH₂, NHNH₂, NHR, N(R₁₀)(R₁₁) (e.g., N(H)CH₃, N(H)CH₂CH₃), R₈—N(R₁₀)(R₁₁) (e.g. CH₂—NH₂); or R₂ and R₅ are joined to form a substituted or unsubstituted 4-8 membered heterocyclic ring (e.g. pyrrolidine);
R₆₀ is absent or is H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₈ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₈ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₈ linear or branched, substituted or unsubstituted alkenyl (e.g., ethenyl), C₂-C₈ linear or branched, substituted or unsubstituted alkynyl (e.g., ethynyl, CC—CH₂-cyclobutyl, CC—CF₃), C₁-C₈ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₈ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₈ linear or branched thioalkoxy, C₁-C₈ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl;
R₇₀ is absent or is H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₈ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₈ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₈ linear or branched, substituted or unsubstituted alkenyl (e.g., ethenyl), C₂-C₈ linear or branched, substituted or unsubstituted alkynyl (e.g., ethynyl, CC—CH₂-cyclobutyl, CC—CF₃), C₁-C₈ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₈ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₈ linear or branched thioalkoxy, C₁-C₈ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl;
R₈₀ is absent or is H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₈ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₈ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₈ linear or branched, substituted or unsubstituted alkenyl (e.g., ethenyl), C₂-C₈ linear or branched, substituted or unsubstituted alkynyl (e.g., ethynyl, CC—CH₂-cyclobutyl, CC—CF₃), C₁-C₈ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₈ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₈ linear or branched thioalkoxy, C₁-C₈ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl;
R₉₀ is absent or is H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₈ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₈ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₈ linear or branched, substituted or unsubstituted alkenyl (e.g., ethenyl), C₂-C₈ linear or branched, substituted or unsubstituted alkynyl (e.g., ethynyl, CC—CH₂-cyclobutyl, CC—CF₃), C₁-C₈ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₈ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₈ linear or branched thioalkoxy, C₁-C₈ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl;
X₁ is O, S, NH, NR (e.g., N—CH₃), S═O, SO₂; or X₁ and R₄ are joined to form a 4-8 membered heterocyclic ring (e.g., pyrrolidine);
X₂, X₃, X₄, X₅ are each independently C or N, wherein if any one of X₂-X₅ is N then the respective R₆₀—R₉₀ is absent;
X₆ is O, NH, NR or CH₂;
G=X is C═O or SO₂ or G(═X)—X₆—R₁ is a tetrazole moiety;
R₈ is [CH₂]p
   wherein p is between 1 and 10;
R₉ is [CH]_{q}, [C]_{q}
   wherein q is between 2 and 10;
R₁₀ and R₁₁ are each independently H, CN, C₁-C₈ linear or branched alkyl (e.g., methyl, ethyl), C(O)R (e.g., C(O)(OCH₃)), or S(O)₂R;
or R₁₀ and R₁₁ are joined to form a substituted or unsubstituted 3-8 membered heterocyclic ring;
R is H, C₁-C₈ linear or branched alkyl (e.g., methyl, ethyl), C₁-C₈ linear or branched alkoxy (e.g., methoxy), phenyl, aryl or heteroaryl, or two gem R substituents are joined together to form a 5 or 6 membered heterocyclic ring;
k is an integer number between 1 and 3 (e.g., 1, 2);
or agrochemically acceptable salt, zwitterion (inner salt), stereoisomer, tautomer, hydrate, N-oxide, reverse amide analog, isotopic variant (e.g., deuterated analog), or any combination thereof.

In various embodiments, if k is not 1, then R₂ is H. In various embodiments, if k is 2, then R₂ is H. In various embodiments, if k is 3, then R₂ is H.

In various embodiments, this invention is directed to a compound represented by the structure of formula I(b):

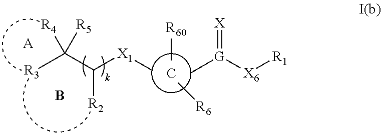

wherein
C ring is a saturated or unsaturated aliphatic or heterocyclic 4-7 membered ring (e.g., cyclohexyl, cyclohexenyl, isoxazole, triazole, pyrazole, oxadiazole, furan, thiazole, pyrrole, thiophene);
   or C is represented by the following structure:

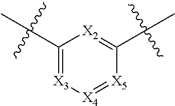

wherein X₂, X₃, X₄ and X₅ are each independently N or C, and
wherein at least one of X₂-X₅ is N;
A and B rings are absent, or are each independently a substituted or unsubstituted aromatic or heteroaromatic ring system (e.g., aryl, pyridine), or a substituted or unsubstituted C₃-C₁₀ cycloalkyl (cyclopropyl, cyclopentyl, cyclohexyl), or a substituted or unsubstituted C₃-C₁₀ heterocyclic ring (e.g., pyrrolidine);
R₁ is H, substituted or unsubstituted C₁-C₁₀ linear or branched, or C₃-C₈ cyclic alkyl (e.g., methyl, ethyl, propyl, butyl, CH₂—CCH, CH₂—COOH, CH₂CH₂COOH), C(O)—R₁₀ (e.g., C(O)—CH₃), R₈—OH (e.g., CH₂—OH), R₈—COO—R₁₀ (e.g., CH₂—COOH, CH₂—COO—CH₃, CH₂—COO—CH₂CH₃, CH₂—CH₂—COOH, CH₂—CH₂—COO—CH₃, CH₂—CH₂—COO—CH₂CH₃), R₈—SH, —R₈—O—R₁₀, (e.g., —CH₂—O—CH₃), R₈—(C₃-C₈ cycloalkyl), R₈-(3-8 membered heterocyclic ring), CF₃, NH₂, NHR, N(R)₂, R₈—N(R₁₀)(Ru), C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₂ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, R₈—N(R₁₀)(R₁₁), C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₃ is H, substituted or unsubstituted C₁-C₁₀ linear or branched, or C₃-C₈ cyclic alkyl (e.g., methyl, ethyl, propyl, CH₂—CCH, C(H)(OH)(CH₃)), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂); C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₂ and R₃ are joined to form a C₃-C₈ substituted or unsubstituted, saturated or unsaturated, aliphatic (e.g. cyclopentyl, cyclohexyl), or aromatic (e.g. pyridine), carbocyclic or heterocyclic ring (e.g. pyrrolidine);
R₄ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂), C(O)H, C₁-C₈ linear or branched C(O)-haloalkyl (e.g., C(O)—CF₃), —C(O)NH₂, C(O)NHR, C(O)N(R₁₀)(Ru), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₃ and R₄ are joined to form a substituted or unsubstituted, saturated or unsaturated, aliphatic or aromatic, carbocyclic or heterocyclic 3-8 membered ring (e.g. cyclopropyl), C═O or ═N—OH;
R₅ is H, OH, SH, NH₂, NHNH₂, NHR, N(R₁₀)(R₁₁) (e.g., N(H)CH₃, N(H)CH₂CH₃), R₈—N(R₁₀)(R₁₁) (e.g. CH₂—NH₂); or R₂ and R₅ are joined to form a substituted or unsubstituted 4-8 membered heterocyclic ring (e.g. pyrrolidine);
R₆ and R₆₀ are each independently H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₈ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₈ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₈ linear or branched, substituted or unsubstituted alkenyl (e.g., ethenyl), C₂-C₈ linear or branched, substituted or unsubstituted alkynyl (e.g., ethynyl, CC—CH₂-cyclobutyl, CC—CF₃), C₁-C₈ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₅ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₈ linear or branched thioalkoxy, C₁-C₈ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl;
X₁ is O, S, NH, NR (e.g., N—CH₃), S═O, SO₂; or X₁ and R₄ are joined to form a 4-8 membered heterocyclic ring (e.g., pyrrolidine);
X₆ is O, NH, NR or CH₂;
G=X is C═O or SO₂ or G(═X)—X₆—R₁ is a tetrazole moiety;
R₈ is [CH₂]p
   wherein p is between 1 and 10;
R₉ is [CH]_{q}, [C]_{q}
   wherein q is between 2 and 10;
R₁₀ and R₁₁ are each independently H, CN, C₁-C₈ linear or branched alkyl (e.g., methyl, ethyl), C(O)R (e.g., C(O)(OCH₃)), or S(O)₂R;
or R₁₀ and R₁₁ are joined to form a substituted or unsubstituted 3-8 membered heterocyclic ring;
R is H, C₁-C₈ linear or branched alkyl (e.g., methyl, ethyl), C₁-C₈ linear or branched alkoxy (e.g., methoxy), phenyl, aryl or heteroaryl, or two gem R substituents are joined together to form a 5 or 6 membered heterocyclic ring;
k is an integer number between 1 and 3 (e.g., 1, 2);
   or agrochemically acceptable salt, zwitterion (inner salt), stereoisomer, tautomer, hydrate, N-oxide, reverse amide analog, isotopic variant (e.g., deuterated analog), or any combination thereof.

In various embodiments, if k is not 1, then R₂ is H. In various embodiments, if k is 2, then R₂ is H. In various embodiments, if k is 3, then R₂ is H.

In various embodiments, ring C is cyclohexane, cyclohexene, isoxazole, triazole, pyrazole, oxadiazole, furan, thiazole, pyrrole or thiophene; each represents a separate embodiment according to this invention.

In various embodiments, ring C is represented by the following structure:

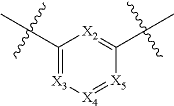

wherein
X₂, X₃, X₄ and X₅ are each independently N or C;

In various embodiments, at least one of X₂-X₅ is N. In various embodiments, at least two of X₂-X₅ are N. In various embodiments, X₂ is N and X₃, X₄ and X₅ are C. In various embodiments, X₂ and X₅ are N and X₃ and X₄ are C. In various embodiments, X₃ and X₅ are N and X₂ and X₄ are C.

In various embodiments, this invention is directed to a compound represented by the structure of formula I(c):

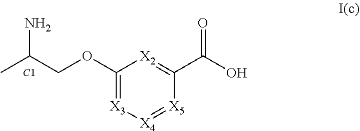

wherein
   each X₂, X₃, X₄ and X₅ is independently N or C; and
   C1 represents a chiral carbon center;
or its agrochemically acceptable salt, zwitterion (inner salt), hydrate, N-oxide, isotopic variant or any combination thereof.

In various embodiments, the compound of formula I(c) is chiral. In various embodiments, the compound of formula I(c) is a substantially pure single enantiomer. In various embodiments, the compound of formula I(c) is the R enantiomer. In various embodiments, compound of formula I(c) is the S enantiomer. In various embodiments, the compound comprises a single enantiomer in a purity of >80%. In various embodiments, the compound comprises a single enantiomer in a purity of >85%. In various embodiments, the compound comprises a single enantiomer in a purity of >90%. In various embodiments, the compound comprises a single enantiomer in a purity of >95%. In various embodiments, the compound comprises a single enantiomer in a purity of >99%.

In various embodiments, C1 of compound of formula I(c) has an R configuration. In various embodiments, C1 has an S configuration.

In various embodiments, at least one of X₂-X₅ of compound of formula I(c) is N. In various embodiments, at least two of X₂-X₅ are N. In various embodiments, X₂ is N and X₃, X₄ and X₅ are C. In various embodiments, X₂ and X₅ are N and X₃ and X₄ are C. In various embodiments, X₃ and X₅ are N and X₂ and X₄ are C.

In various embodiments, this invention is directed to a compound represented by the structure of formula I(d):

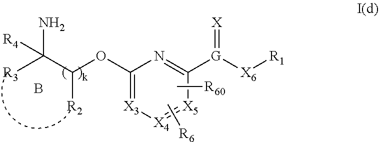

wherein
B ring is absent, or is a substituted or unsubstituted aromatic or heteroaromatic ring system (e.g., aryl, pyridine), or a substituted or unsubstituted C₃-C₁₀ cycloalkyl (cyclopropyl, cyclopentyl, cyclohexyl), or a substituted or unsubstituted C₃-C₁₀ heterocyclic ring (e.g., pyrrolidine);
R₁ is H, substituted or unsubstituted C₁-C₁₀ linear or branched, or C₃-C₈ cyclic alkyl (e.g., methyl, ethyl, propyl, butyl, CH₂—CCH, CH₂—COOH, CH₂CH₂COOH), C(O)—R₁₀ (e.g., C(O)—CH₃), R₈—OH (e.g., CH₂—OH), R₈—COO—R₁₀ (e.g., CH₂—COOH, CH₂—COO—CH₃, CH₂—COO—CH₂CH₃, CH₂—CH₂—COOH, CH₂—CH₂—COO—CH₃, CH₂—CH₂—COO—CH₂CH₃), R₈—SH, —R₈—O—R₁₀, (e.g., —CH₂—O—CH₃), R₈—(C₃-C₈ cycloalkyl), R₈-(3-8 membered heterocyclic ring), CF₃, NH₂, NHR, N(R)₂, R₈—N(R₁₀)(R₁₁), C(O)H, C₁-C₈ linear or branched C(O)-haloalkyl (e.g., C(O)—CF₃), —C(O)NH₂, C(O)NHR, C(O)N(R₁₀)(Ru), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₂ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, R₈—N(R₁₀)(R₁₁), C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₃ is H, substituted or unsubstituted C₁-C₁₀ linear or branched, or C₃-C₈ cyclic alkyl (e.g., methyl, ethyl, propyl, CH₂—CCH, C(H)(OH)(CH₃)), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂); C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₂ and R₃ are joined to form a C₃-C₈ substituted or unsubstituted, saturated or unsaturated, aliphatic (e.g. cyclopropyl, cyclopentyl, cyclohexyl), or aromatic (e.g. pyridine), carbocyclic or heterocyclic ring (e.g. pyrrolidine);
R₄ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂), C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₃ and R₄ are joined to form a substituted or unsubstituted, saturated or unsaturated, aliphatic or aromatic, carbocyclic or heterocyclic 3-8 membered ring (e.g. cyclopropyl), C═O or ═N—OH;
R₆ and R₆₀ are each independently H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₈ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₈ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₈ linear or branched, substituted or unsubstituted alkenyl (e.g., ethenyl), C₂-C₈ linear or branched, substituted or unsubstituted alkynyl (e.g., ethynyl, CC—CH₂-cyclobutyl, CC—CF₃), C₁-C₈ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₈ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₈ linear or branched thioalkoxy, C₁-C₈ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl;
X₃, X₄, X₅ are each independently C or N;
X₆ is O, NH, NR or CH₂;
G=X is C═O or SO₂ or G(═X)—X₆—R₁ is a tetrazole moiety;
R₈ is [CH₂]p
   wherein p is between 1 and 10;
R₉ is [CH]_{q}, [C]_{q}
   wherein q is between 2 and 10;
R₁₀ and R₁₁ are each independently H, CN, C₁-C₈ linear or branched alkyl (e.g., methyl, ethyl), C(O)R (e.g., C(O)(OCH₃)), or S(O)₂R;
or R₁₀ and R₁₁ are joined to form a substituted or unsubstituted 3-8 membered heterocyclic ring;
R is H, C₁-C₅ linear or branched alkyl (e.g., methyl, ethyl), C₁-C₅ linear or branched alkoxy (e.g., methoxy), phenyl, aryl or heteroaryl, or two gem R substituents are joined together to form a 5 or 6 membered heterocyclic ring;
k is an integer number between 1 and 3 (e.g., 1, 2);
   or agrochemically acceptable salt, zwitterion (inner salt), stereoisomer, tautomer, hydrate, N-oxide, reverse amide analog, isotopic variant (e.g., deuterated analog), or any combination thereof.

In various embodiments, if k is not 1, then R₂ is H. In various embodiments, if k is 2, then R₂ is H. In various embodiments, if k is 3, then R₂ is H.

In various embodiments, this invention is directed to a compound represented by the structure of formula I(e):

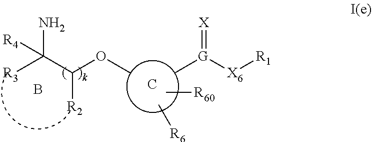

wherein
B ring is absent, or is a substituted or unsubstituted aromatic or heteroaromatic ring system (e.g., aryl, pyridine), or a substituted or unsubstituted C₃-C₁₀ cycloalkyl (cyclopropyl, cyclopentyl, cyclohexyl), or a substituted or unsubstituted C₃-C₁₀ heterocyclic ring (e.g., pyrrolidine); C ring is a saturated or unsaturated, 5 membered heterocyclic ring (e.g., isoxazole, triazole, pyrazole, oxadiazole, furan, thiazole, pyrrole, thiophene), a cyclohexane or a cyclohexene ring;
R₁ is H, substituted or unsubstituted C₁-C₁₀ linear or branched, or C₃-C₈ cyclic alkyl (e.g., methyl, ethyl, propyl, butyl, CH₂—CCH, CH₂—COOH, CH₂CH₂COOH), C(O)—R₁₀ (e.g., C(O)—CH₃), R₈—OH (e.g., CH₂—OH), R₈—COO—R₁₀ (e.g., CH₂—COOH, CH₂—COO—CH₃, CH₂—COO—CH₂CH₃, CH₂—CH₂—COOH, CH₂—CH₂—COO—CH₃, CH₂—CH₂—COO—CH₂CH₃), R₈—SH, —R₈—O—R₁₀, (e.g., —CH₂—O—CH₃), R₈—(C₃-C₈ cycloalkyl), R₈-(3-8 membered heterocyclic ring), CF₃, NH₂, NHR, N(R)₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
R₂ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₃ is H, substituted or unsubstituted C₁-C₁₀ linear or branched, or C₃-C₈ cyclic alkyl (e.g., methyl, ethyl, propyl, CH₂—CCH, C(H)(OH)(CH₃)), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂); C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₂ and R₃ are joined to form ring B as described above;
R₄ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂), C(O)H, C₁-C₈ linear or branched C(O)-haloalkyl (e.g., C(O)—CF₃), —C(O)NH₂, C(O)NHR, C(O)N(R₁₀)(R₁₁), C₂-C₈ linear or branched, substituted or unsubstituted alkenyl (e.g., CH═C(Ph)₂), C₁-C₈ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl or R₃ and R₄ are joined to form a substituted or unsubstituted, saturated or unsaturated, aliphatic or aromatic, carbocyclic or heterocyclic 3-8 membered ring (e.g. cyclopropyl), C═O or ═N—OH;
R₆ and R₆₀ are each independently H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₈ linear or branched C(O)-haloalkyl (e.g., C(O)—CF₃), —C(O)NH₂, C(O)NHR, C(O)N(R₁₀)(Ru) (e.g., C(O)N(CH₃)₂), SO₂R, SO₂N(R₁₀)(R₁₁) (e.g., SO₂N(CH₃)₂, SO₂NHC(O)CH₃), C₁-C₅ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₅ linear or branched, substituted or unsubstituted alkenyl (e.g., ethenyl), C₂-C₅ linear or branched, substituted or unsubstituted alkynyl (e.g., ethynyl, CC—CH₂-cyclobutyl, CC—CF₃), C₁-C₈ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₅ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₅ linear or branched thioalkoxy, C₁-C₅ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl,
X₆ is O, NH, NR or CH₂;
G=X is C═O or SO₂ or G(═X)—X₆—R₁ is a tetrazole moiety;
R₈ is [CH₂]p
   wherein p is between 1 and 10;
R₉ is [CH]_{q}, [C]_{q}
   wherein q is between 2 and 10;
R₁₀ and R₁₁ are each independently H, CN, C₁-C₅ linear or branched alkyl (e.g., methyl, ethyl), C(O)R (e.g., C(O)(OCH₃)), or S(O)₂R;
or R₁₀ and R₁₁ are joined to form a substituted or unsubstituted 3-8 membered heterocyclic ring;
R is H, C₁-C₅ linear or branched alkyl (e.g., methyl, ethyl), C₁-C₅ linear or branched alkoxy (e.g., methoxy), phenyl, aryl or heteroaryl, or two gem R substituents are joined together to form a 5 or 6 membered heterocyclic ring; and
k is an integer number between 1 and 3;
   or its agrochemically acceptable salt, zwitterion (inner salt), stereoisomer, tautomer, hydrate, N-oxide, reverse amide analog, isotopic variant (e.g., deuterated analog), or any combination thereof.

In various embodiments, if k is not 1, then R₂ is H. In various embodiments, if k is 2, then R₂ is H. In various embodiments, if k is 3, then R₂ is H.

In various embodiments, compound of formula I, or I(a)-I(e) is not: 6-(2-aminopropoxy)picolinic acid. In various embodiments, the compound is not 6-(2-aminopropoxy)pyrazine-2-carboxylic acid. In various embodiments, the compound is not 2-(2-aminopropoxy)pyrimidine-4-carboxylic acid In various embodiments, the compound is not 6-(2-aminopropoxy)-3-fluoropicolinic acid. In various embodiments, the compound is not 6-(2-aminopropoxy)-4-fluoropicolinic acid. In various embodiments, the compound is not 6-(2-aminobutoxy)picolinic acid. In various embodiments, the compound is not 6-((2-aminocyclopentyl)oxy)picolinic acid. In various embodiments, the compound is not methyl 6-(2-aminopropoxy)picolinate. In various embodiments, the compound is not ethyl 6-(2-aminopropoxy)picolinate.

It is understood that the compounds according to this invention may be also present in their inner salt form (i.e., zwitterion). Accordingly, in solution a chemical equilibrium will be established between the “parent” molecule and its zwitterionic form.

In various embodiments, the compound is an herbicide. In various embodiments, the compound is an herbicidal compound. In various embodiments, the compound is a pesticide. In various embodiments, the compound is a pesticidal compound.

In various embodiments, this invention is directed to a compound represented by any one of the following structures:

TABLE 1

| Compound | |
|---|---|
| Number | Structure |
| 101 | 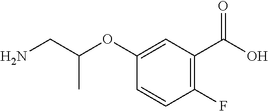 |
| 102 | 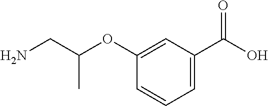 |
| 103 | 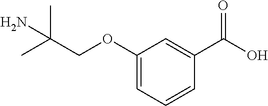 |
| 104 | 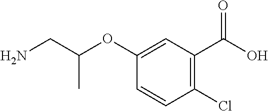 |
| 105 | 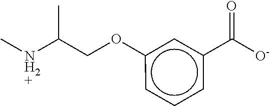 |
| 106 | 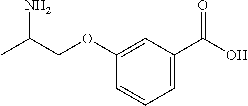 |
| 107 | 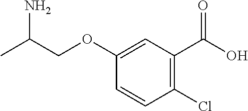 |
| 108 | 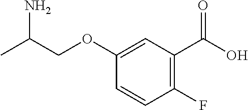 |
| 109 | 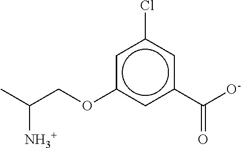 |
| 110 | 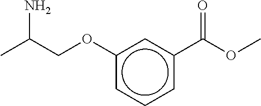 |
| 111 | 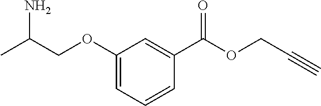 [SMILES: C#CCOC(=O)c1cccc(OCC(C)N)c1] |
| 112 | 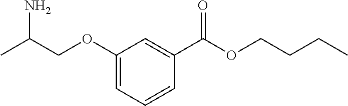 [SMILES: CCCCOC(=O)c1cccc(OCC(C)N)c1] |
| 113 | 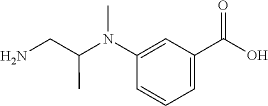 |
| 114 | 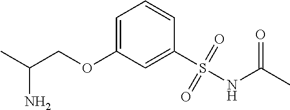 [SMILES: CC(=O)NS(=O)(=O)c1cccc(OCC(C)N)c1] |
| 115 | 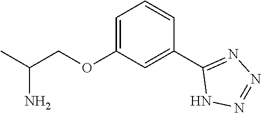 [SMILES: CC(N)COc1cccc(-c2nnn[nH]2)c1] |
| 116 | 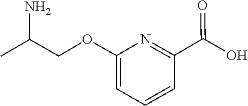 |
| 117 | 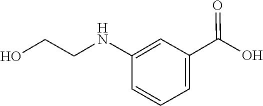 [SMILES: O=C(O)c1cccc(NCCO)c1] |
| 118 | 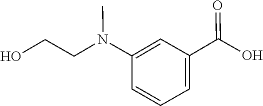 |
| 119 | 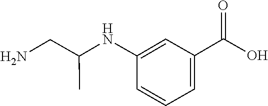 |
| 120 | 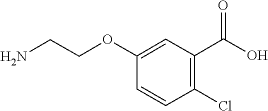 [SMILES: NCCOc1ccc(Cl)c(C(=O)O)c1] |
| 121 | 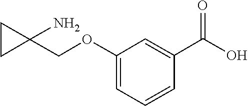 [SMILES: NC1(COc2cccc(C(=O)O)c2)CC1] |
| 122 | 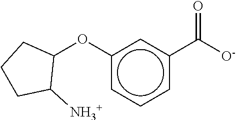 |
| 123 | 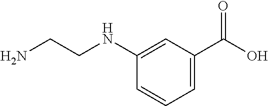 [SMILES: NCCNc1cccc(C(=O)O)c1] |
| 124 | 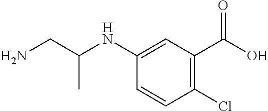 |
| 125 | 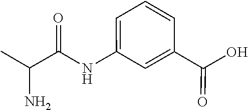 |
| 126 | 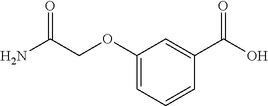 [SMILES: NC(=O)COc1cccc(C(=O)O)c1] |
| 127 | 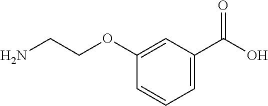 [SMILES: NCCOc1cccc(C(=O)O)c1] |
| 128 | 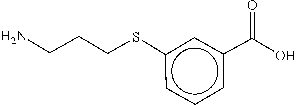 |
| 129 | 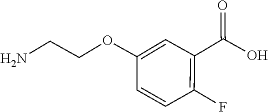 [SMILES: NCCOc1ccc(F)c(C(=O)O)c1] |
| 130 | 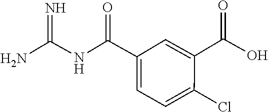 [SMILES: N=C(N)NC(=O)c1ccc(Cl)c(C(=O)O)c1] |
| 131 | 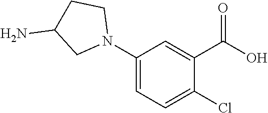 [SMILES: NC1CCN(c2ccc(Cl)c(C(=O)O)c2)C1] |
| 132 | 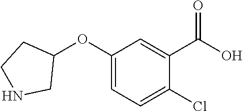 [SMILES: O=C(O)c1cc(OC2CCNC2)ccc1Cl] |
| 133 | 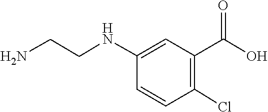 [SMILES: NCCNc1ccc(Cl)c(C(=O)O)c1] |
| 134 | 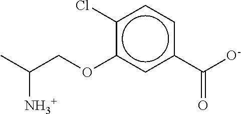 |
| 135 | 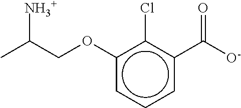 |
| 136 | 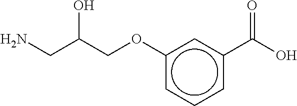 |
| 137 | 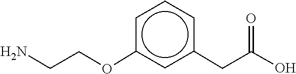 |
| 138 | 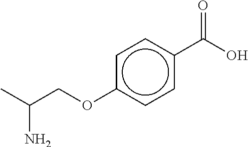 |
| 139 | 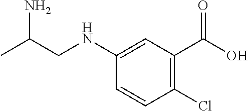 |
| 140 | 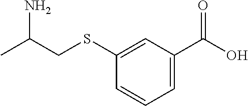 |
| 141 | 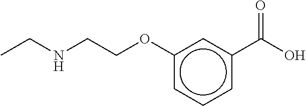 |
| 142 | 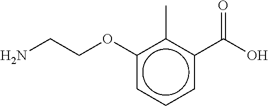 |
| 143 | 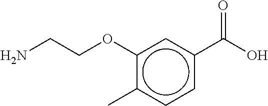 |
| 144 | 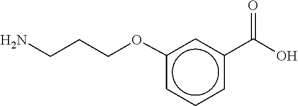 |
| 145 | 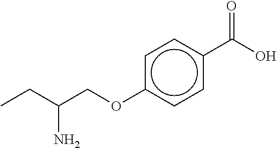 |
| 146 | 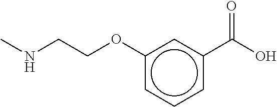 |
| 147 | 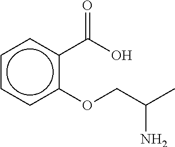 |
| 148 | 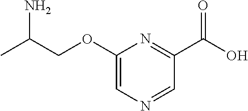 |
| 149 | 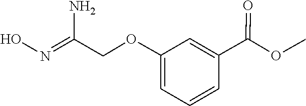 [SMILES: COC(=O)c1cccc(OC/C(N)=N/O)c1] |
| 150 | 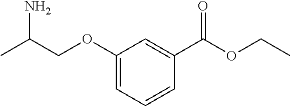 [SMILES: CCOC(=O)c1cccc(OCC(C)N)c1] |
| 151 | 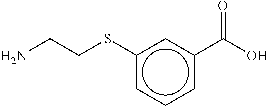 |
| 152 | 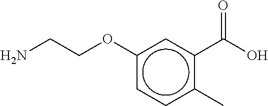 |
| 153 | 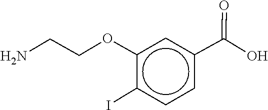 |
| 154 | 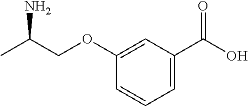 |
| 155 | 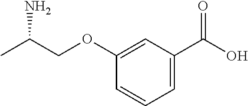 |
| 156 | 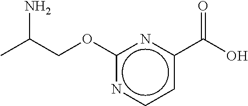 |
| 157 | 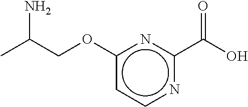 |
| 158 | 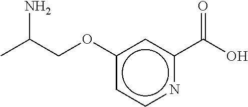 |
| 159 | 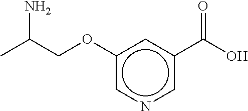 |
| 160 | 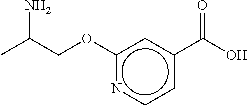 |
| 161 | 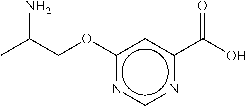 |
| 162 | 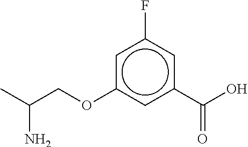 |
| 163 | 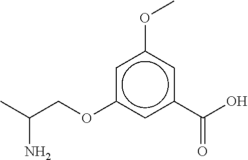 |
| 164 | 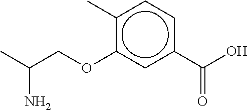 |
| 165 | 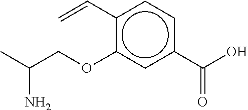 |
| 166 | 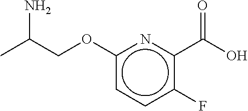 |
| 167 | 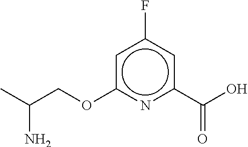 |
| 168 | 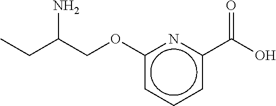 |
| 169 | 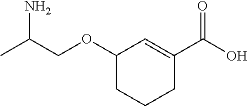 |
| 170 | 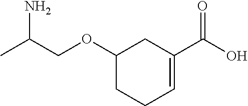 |
| 171 | 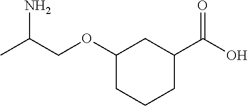 |
| 172 | 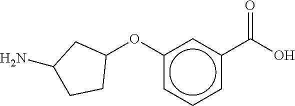 |
| 173 | 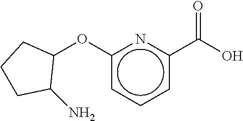 |
| 174 | 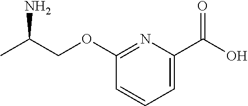 |
| 175 | 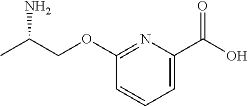 |
| 176 | 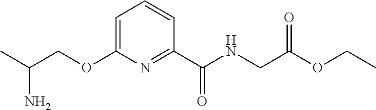 [SMILES: CCOC(=O)CNC(=O)c1cccc(OCC(C)N)n1] |
| 177 | 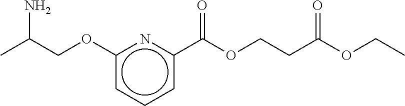 |
| 178 | 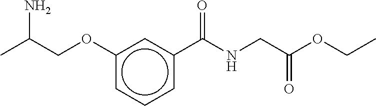 |
| 179 | 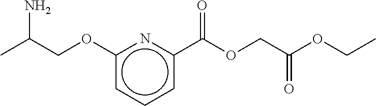 |
| 180 | 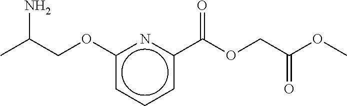 |
| 181 | 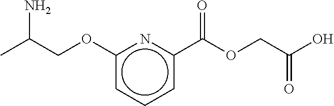 |
| 182 | 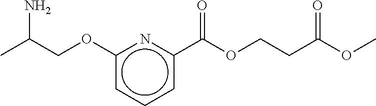 |
| 183 | 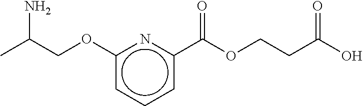 |
| 184 | 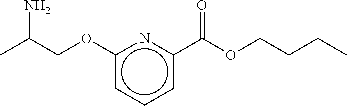 |
| 185 | 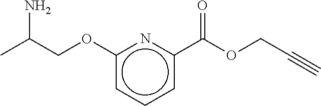 |
| 186 | 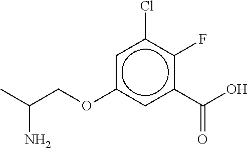 |
| 187 | 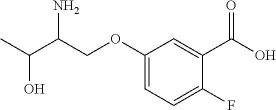 [SMILES: CC(O)C(N)COc1ccc(F)c(C(=O)O)c1] |
| 188 | 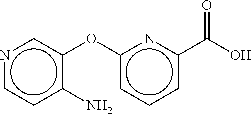 |
| 189 | 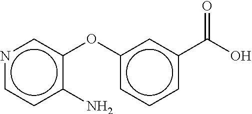 |
| 190 | 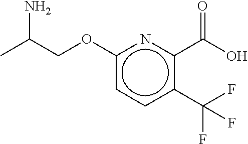 |
| 191 | 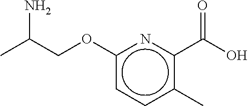 |
| 192 | 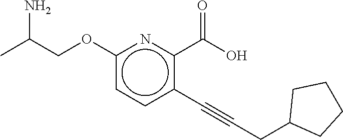 |
| 193 | 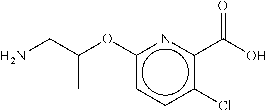 |
| 194 | 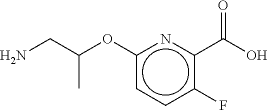 |
| 195 | 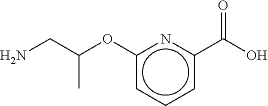 |
| 196 | 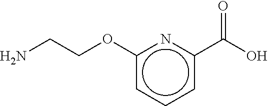 |
| 197 | 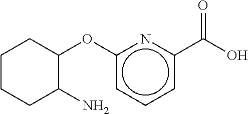 |
| 198 | 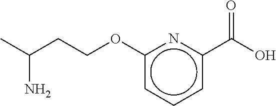 |
| 199 | 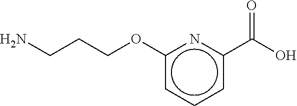 |
| 200 | 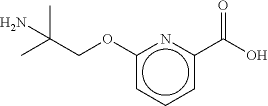 |
| 201 | 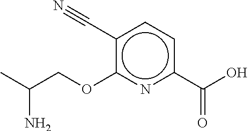 |
| 202 | 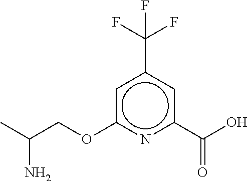 |
| 203 | 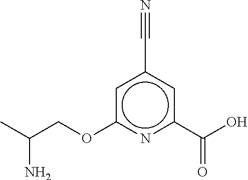 |
| 204 | 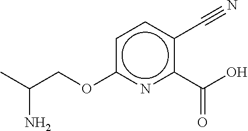 |
| 205 | 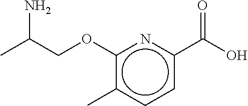 |
| 206 | 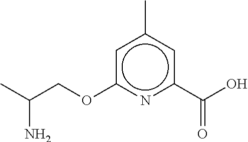 |
| 207 | 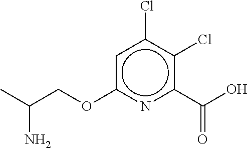 |
| 208 | 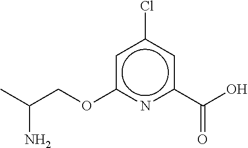 |
| 209 | 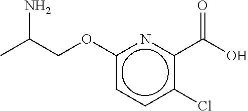 |
| 210 | 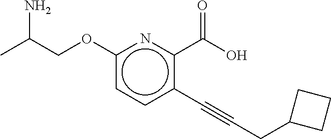 |
| 211 | 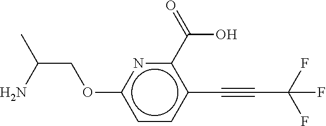 |
| 212 | 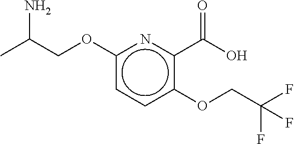 |
| 213 | 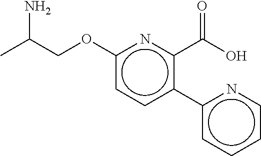 |
| 214 | 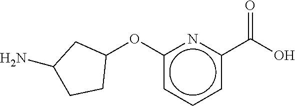 |
| 215 | 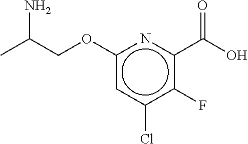 |
| 216 | 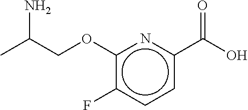 |
| 218 | 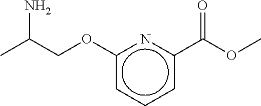 |
| 219 | 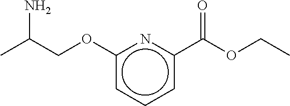 |
| 400 | 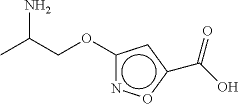 |
| 401 | 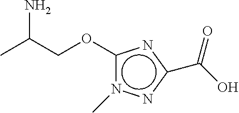 |
| 402 | 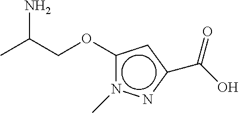 |
| 403 | 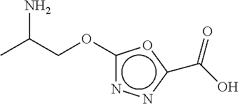 |
| 404 | 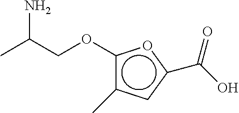 |
| 405 | 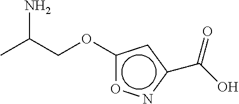 |
| 406 | 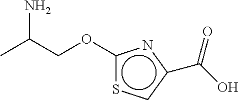 |
| 407 | 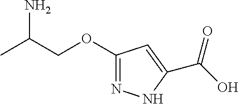 |
| 408 | 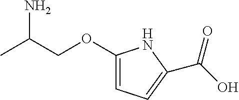 |
| 409 | 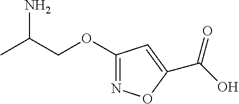 |
| 410 | 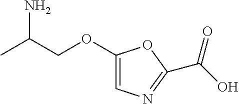 |
| 411 | 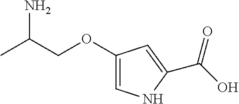 |
| 412 | 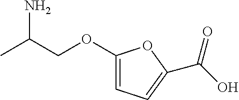 |
| 413 | 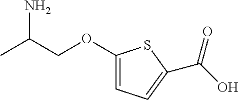 |
| 414 | 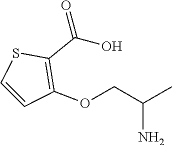 |
| 415 | 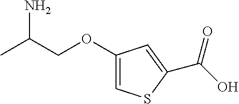 |

In various embodiments, the compound is an herbicide. In various embodiments, the compound is a pesticide. In various embodiments, the compound is herbicidal compound. In various embodiments, the compound is pesticidal compound.

In various embodiments, this invention is directed to a compound represented by any one of the following structures:

TABLE 2

| Compound | |
|---|---|
| Number | Structure |
| 157 | 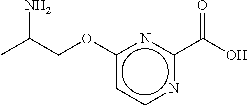 |
| 161 | 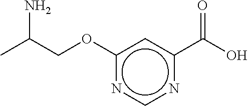 |
| 169 | 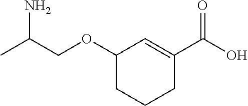 |
| 170 | 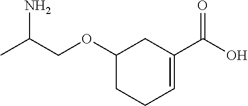 |
| 171 | 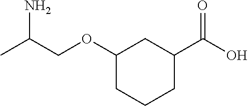 |
| 174 | 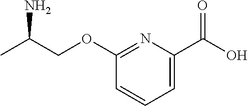 |
| 175 | 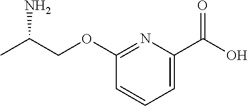 |
| 176 | 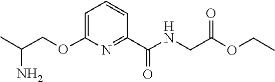 [SMILES: CCOC(=O)CNC(=O)c1cccc(OCC(C)N)n1] |
| 177 | 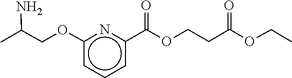 |
| 179 | 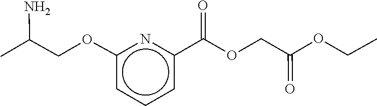 |
| 180 | 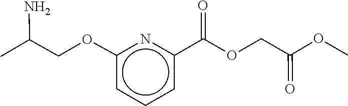 |
| 181 | 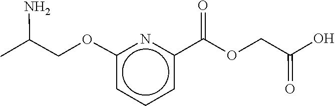 |
| 182 | 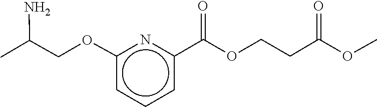 |
| 183 | 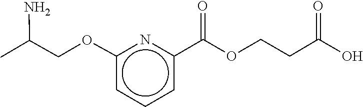 |
| 184 | 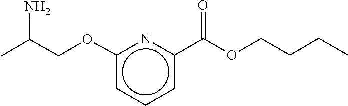 |
| 185 | 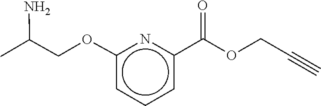 |
| 188 | 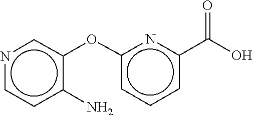 |
| 190 | 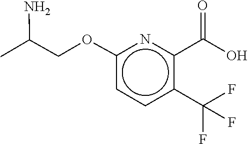 |
| 191 | 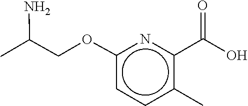 |
| 192 | 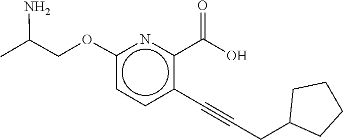 |
| 193 | 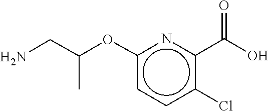 |
| 194 | 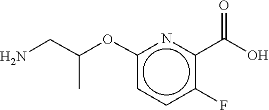 |
| 195 | 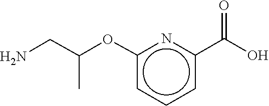 |
| 196 | 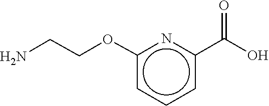 |
| 197 | 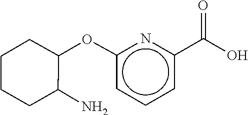 |
| 198 | 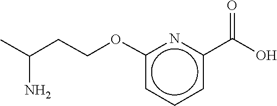 |
| 199 | 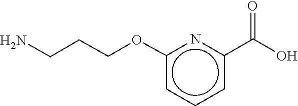 |
| 200 | 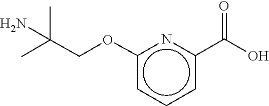 |
| 201 | 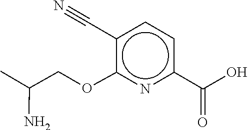 |
| 202 | 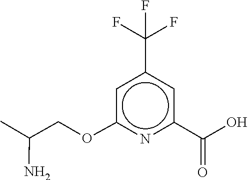 |
| 203 | 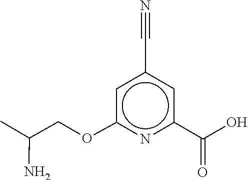 |
| 204 | 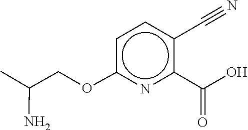 |
| 205 | 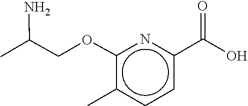 |
| 206 | 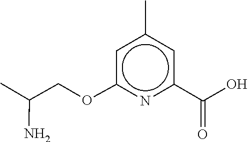 |
| 207 | 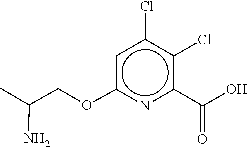 |
| 208 | 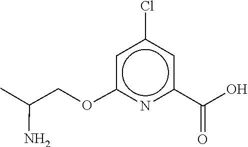 |
| 209 | 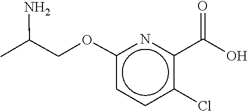 |
| 210 | 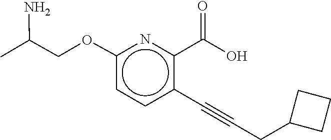 |
| 211 | 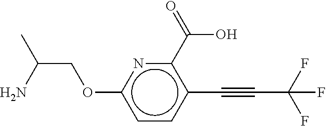 |
| 212 | 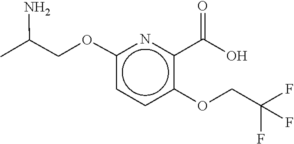 |
| 213 | 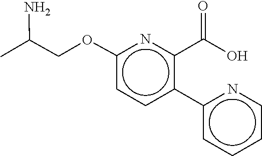 |
| 215 | 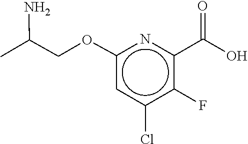 |
| 216 | 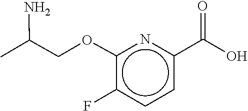 |

In various embodiments, the compound is an herbicide. In various embodiments, the compound is a pesticide. In various embodiments, the compound is herbicidal compound. In various embodiments, the compound is pesticidal compound.

In various embodiments, this invention is directed to a use of a compound represented by any one of the structures of Tables 1 or 2, or an agrochemical composition thereof, in controlling the growth of undesired plants; each structure represents a separate embodiment according to this invention.

In some embodiments, A of formula I, I(a), I(b), and/or I(c) is absent. In some embodiments, A is a substituted or unsubstituted single or fused aromatic or heteroaromatic ring system, or a substituted or unsubstituted single or fused C₃-C₁₀ cycloalkyl, or a substituted or unsubstituted single or fused C₃-C₁₀ heterocyclic ring; each is a separate embodiment according to this invention. In some embodiments, A is a single aromatic ring system. In some embodiments, A is a single heteroaromatic ring. In some embodiments, A is a single C₃-C₁₀ cycloalkyl. In some embodiments, A is cyclopropyl. In some embodiments, A is a single C₃-C₁₀ heterocyclic ring. In some embodiments, A is a fused aromatic ring system. In some embodiments, A is a fused heteroaromatic ring system. In some embodiments, A is a fused C₃-C₁₀ cycloalkyl. In some embodiments, A is a fused C₃-C₁₀ heterocyclic ring system. In other embodiments, A is cyclobutyl, cyclopentyl, cyclohexyl, cycloheptyl; each represents a separate embodiment according to this invention. In some embodiments, A ring may be further substituted, with at least one substituent selected from: C(O)—CH₃, C₁-C₅ linear or branched, substituted or unsubstituted alkyl, methyl, ethyl, propyl, iso-propyl, t-Bu, iso-butyl, pentyl, benzyl, F, Cl, Br, I, OH, SH, C₁-C₅ linear or branched alkyl, C₃-C₈ cycloalkyl (e.g., cyclopentyl), C₂-C₅ linear or branched alkenyl (e.g., ethenyl), C₂-C₅ linear or branched alkynyl, alkyl amide (i.e., C(O)NH—R or NHC(O)—R), linear, branched or cyclic alkoxy, COOH, alkyl ester (i.e., COOR or OC(O)—R), OH, COO(R), N(R)₂, NH₂, CF₃, aryl, phenyl, heteroaryl, R₈—(C₃-C₈ cycloalkyl), halophenyl, CN, NO₂ or any combination thereof; each represents a separate embodiment according to this invention.

In some embodiments, B of formula I, I(a), I(b), I(c), I(d) and/or I(e) is absent. In some embodiments, ring B is a substituted or unsubstituted single or fused aromatic or heteroaromatic ring system, or a substituted or unsubstituted single or fused C₃-C₁₀ cycloalkyl, or a substituted or unsubstituted single or fused C₃-C₁₀ heterocyclic ring; each represents a separate embodiment according to this invention. In some embodiments, B is a single aromatic ring system (i.e., arene). In some embodiments, B is a single heteroaromatic ring (e.g., pyridine). In some embodiments, B is a single C₃-C₁₀ cycloalkyl (e.g., cyclopropyl, cyclopentyl, cyclohexyl). In some embodiments, B is a single C₃-C₁₀ heterocyclic ring (e.g., pyrrolidinyl). In some embodiments, B is a fused aromatic ring system. In some embodiments, B is a fused heteroaromatic ring system. In some embodiments, B is a fused C₃-C₁₀ cycloalkyl. In some embodiments, B is a fused C₃-C₁₀ heterocyclic ring system. In some embodiments, B is a single C₃-C₁₀ cycloalkyl. In some embodiments, B is cyclopentyl. In some embodiments, B is cyclohexyl. In some embodiments, B is cyclopropyl. In some embodiments, B may be further substituted, with at least one substituent selected from: F, Cl, Br, I, OH, SH, R₈—OH, R₈—SH, —R₈—O—R₁₀, CF₃, CN, NO₂, NH₂, NHR, N(R)₂, R₈—N(R₁₀)(R₁₁), —OC(O)CF₃, —OCH₂Ph, NHC(O)OBz, —NHC(O)—R₁₀, COOH, —C(O)Ph, C(O)O—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), C₁-C₅ linear or branched or C₃-C₈ cyclic haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted 3-8 membered heterocyclic ring, substituted or unsubstituted aryl, C(O)—CH₃, C₁-C₅ linear or branched, substituted or unsubstituted alkyl, methyl, ethyl, propyl, iso-propyl, t-Bu, iso-butyl, pentyl, benzyl, C₂-C₅ linear or branched alkenyl (e.g., ethenyl), C₂-C₅ linear or branched alkynyl, alkyl ester (i.e., COOR or OC(O)—R), aryl, phenyl, R₈—(C₃-C₈ cycloalkyl), and halophenyl; each represents a separate embodiment according to this invention.

In some embodiments, C ring of formula I(b) or I(e) is a saturated or unsaturated, 5 membered heterocyclic ring. In some embodiments, C is isoxazole. In some embodiments, C is triazole. In some embodiments, C is pyrazole. In some embodiments, C is oxadiazole. In some embodiments, C is furan. In some embodiments, C is thiazole. In some embodiments, C is pyrrole. In some embodiments, C is thiophene. In some embodiments, C is isoxazole, triazole, pyrazole, oxadiazole, furan, thiazole, pyrrole, thiophene; each represents a separate embodiment according to this invention. In some embodiments, C ring is cyclohexane. In some embodiments, C ring is cyclohexene. In some embodiments, C ring is represented by any one of the following structures:

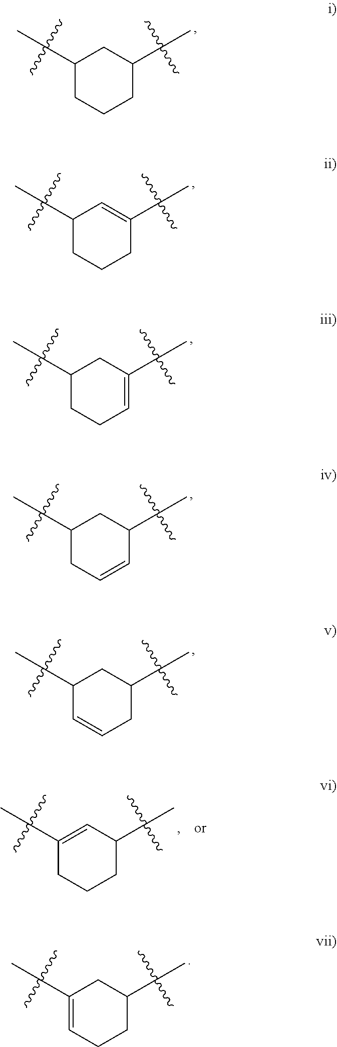

In some embodiments, C ring is represented by the following structure:

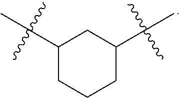 [SMILES: "." ; *C1CCCC(*)C1]

In some embodiments, C ring is represented by the following structure:

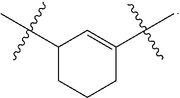 [SMILES: "." ; *C1=CC(*)CCC1]

In some embodiments, C ring is represented by the following structure:

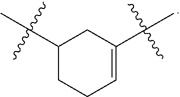 [SMILES: "." ; *C1=CCCC(*)C1]

In some embodiments, C ring is represented by the following structure:

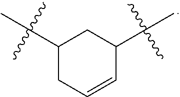 [SMILES: "." ; *C1C=CCC(*)C1]

In some embodiments, C ring is represented by the following structure:

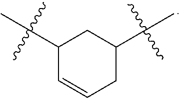 [SMILES: "." ; *C1C=CCC(*)C1]

In some embodiments, C ring is represented by the following structure:

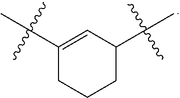 [SMILES: "." ; *C1=CC(*)CCC1]

In some embodiments, C ring is represented by the following structure:

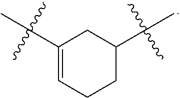 [SMILES: "." ; *C1=CCCC(*)C1]

In some embodiments, C ring is represented by the following structure:

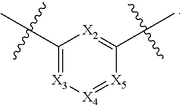

wherein X₂, X₃, X₄ and X₅ are each independently N or C. In various embodiments, at least one of X₂-X₅ is N. In various embodiments, at least two of X₂-X₅ are N. In various embodiments, X₂ is N and X₃, X₄ and X₅ are C. In various embodiments, X₂ and X₅ are N and X₃ and X₄ are C. In various embodiments, X₃ and X₅ are N and X₂ and X₄ are C.

In some embodiments, R₁ of compound of formula I and/or I(a)-I(e) is H. In some embodiments, R₁ is substituted or unsubstituted C₁-C₁₀ linear or branched, or C₃-C₈ cyclic alkyl. In some embodiments, R₁ is unsubstituted C₁-C₁₀ linear or branched alkyl. In some embodiments, R₁ is methyl. In some embodiments, R₁ is ethyl. In some embodiments, R₁ is propyl. In some embodiments, R₁ is iso-propyl. In some embodiments, R₁ is butyl. In some embodiments, R₁ is propyl, butyl, t-Bu, iso-butyl, pentyl, benzyl; each represents a separate embodiment according to this invention. In some embodiments, R₁ is substituted C₁-C₁₀ linear or branched alkyl. In some embodiments, R₁ is CH₂—CCH. In some embodiments, R₁ is CH₂—COOH. In some embodiments, R₁ is CH₂CH₂COOH. In some embodiments, R₁ is substituted or unsubstituted C₃-C₈ cyclic alkyl. In some embodiments, R₁ is C(O)—R₁₀. In some embodiments, R₁ is C(O)—CH₃. In some embodiments, R₁ is R₈—OH. In some embodiments, R₁ is CH₂—OH. In some embodiments, R₁ is R₈—COO—R₁₀. In some embodiments, R₁ is CH₂—COOH. In some embodiments, R₁ is CH₂—COO—CH₃. In some embodiments, R₁ is CH₂—COO—CH₂CH₃. In some embodiments, R₁ is CH₂—CH₂—COOH. In some embodiments, R₁ is CH₂—CH₂—COO—CH₃. In some embodiments, R₁ is CH₂—CH₂—COO—CH₂CH₃. R₈—SH, —R₈—O—R₁₀, —CH₂—O—CH₃, R₈—(C₃-C₈ cycloalkyl), R₈-(3-8 membered heterocyclic ring), CF₃, NH₂, NHR, N(R)₂, R₈—N(R₁₀)(R₁₁), C(O)H, C₁-C₅ linear or branched C(O)-haloalkyl, C(O)—CF₃, —C(O)NH₂, C(O)NHR, C(O)N(R₁₀)(R₁₁), C₂-C₅ linear or branched, substituted or unsubstituted alkenyl, CH═C(Ph)₂, C₁-C₅ linear or branched or C₃-C₈ cyclic haloalkyl, substituted or unsubstituted 3-8 membered heterocyclic ring, substituted or unsubstituted aryl, phenyl, or substituted or unsubstituted benzyl; each represents a separate embodiment according to this invention. In some embodiments, R₁ is H, substituted or unsubstituted C₁-C₁₀ linear or branched, or C₃-C₅ cyclic alkyl, C(O)—R₁₀, R₈—OH, R₈—COO—R₁₀, or C₂-C₅ linear or branched alkenyl. In some embodiments, the alkyl is methyl, ethyl, propyl, butyl, CH₂—CCH, CH₂—COOH or CH₂CH₂COOH. In some embodiments, the C(O)—R₁₀ is C(O)—CH₃. In some embodiments, the alkenyl is ethenyl. In some embodiments, the R₈—OH is CH₂—OH. In some embodiments, the R₈—COO—R₁₀ is CH₂—COOH, CH₂—COO—CH₃, CH₂—COO—CH₂CH₃, CH₂—CH₂—COOH, CH₂—CH₂—COO—CH₃ or CH₂—CH₂—COO—CH₂CH₃. In some embodiments, R₁ may be further substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiments, R₂ of compound of formula I and/or I(a)-I(e) is H. In some embodiments, R₂ is substituted or unsubstituted C₁-C₁₀ linear or branched, or C₃-C₈ cyclic alkyl. In some embodiments, R₂ is unsubstituted C₁-C₁₀ linear or branched alkyl. In some embodiments, R₂ is methyl, ethyl, propyl, iso-propyl, butyl, t-Bu, iso-butyl, pentyl, benzyl; each represents a separate embodiment according to this invention. In some embodiments, R₂ is substituted C₁-C₁₀ linear or branched alkyl. In some embodiments, R₂ is substituted or unsubstituted C₃-C₈ cyclic alkyl. In some embodiments, R₂ is F, Cl, Br or I; each represents a separate embodiment according to this invention. In some embodiments, R₂ is OH, SH, R₈—OH, CH₂—OH, R₈—SH, —R₈—O—R₁₀, —CH₂—O—CH₃, R₈—(C₃-C₈ cycloalkyl), R₈-(3-8 membered heterocyclic ring), CF₃, CN, NO₂, R₈—N(R₁₀)(R₁₁), C(O)H, C₁-C₅ linear or branched C(O)-haloalkyl, C(O)—CF₃, —C(O)NH₂, C(O)NHR, C(O)N(R₁₀)(R₁₁), C₂-C₅ linear or branched, substituted or unsubstituted alkenyl, CH═C(Ph)₂, C₁-C₅ linear or branched or C₃-C₈ cyclic haloalkyl, substituted or unsubstituted 3-8 membered heterocyclic ring, substituted or unsubstituted aryl, phenyl or substituted or unsubstituted benzyl; each represents a separate embodiment according to this invention. In some embodiments, R₂ is H, substituted or unsubstituted C₁-C₁₀ linear or branched, or C₃-C₈ cyclic alkyl. In some embodiments, R₂ may be further substituted with at least one substitution selected from F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiments, R₃ of compound of formula I and/or I(a)-I(e) is H. In some embodiments, R₃ is substituted or unsubstituted C₁-C₁₀ linear or branched, or C₃-C₈ cyclic alkyl. In some embodiments, R₃ is unsubstituted C₁-C₁₀ linear or branched alkyl. In some embodiments, R₃ is methyl, ethyl, propyl, iso-propyl, butyl, t-Bu, iso-butyl, pentyl, benzyl. In some embodiments, R₃ is a substituted C₁-C₁₀ linear or branched alkyl. In some embodiments, R₃ is CH₂—CCH, C(H)(OH)(CH₃); each represents a separate embodiment according to this invention. In some embodiments, R₃ is substituted or unsubstituted C₃-C₈ cyclic alkyl. In some embodiments, R₃ is R₈—N(R₁₀)(R₁₁). In some embodiments, R₃ is CH₂—NH₂. In some embodiments, R₃ is F, Cl, Br, I, OH, SH, R₈—OH, CH₂—OH, R₈—SH, —R₈—O—R₁₀, —CH₂—O—CH₃, R₈—(C₃-C₈ cycloalkyl), R₈-(3-8 membered heterocyclic ring), CF₃, CN, NO₂, NH₂, NHR, N(R)₂, C(O)H, C₁-C₅ linear or branched C(O)-haloalkyl, C(O)—CF₃, —C(O)NH₂, C(O)NHR, C(O)N(R₁₀)(R₁₁), C₂-C₅ linear or branched, substituted or unsubstituted alkenyl, C₁-C₅ linear or branched or C₃-C₈ cyclic haloalkyl, substituted or unsubstituted 3-8 membered heterocyclic ring, substituted or unsubstituted aryl, phenyl, or substituted or unsubstituted benzyl; each represents a separate embodiment according to this invention. In some embodiments, R₃ is H, substituted or unsubstituted C₁-C₁₀ linear or branched, or C₃-C₈ cyclic alkyl, or R₈—N(R₁₀)(R₁₁). In some embodiments, R₃ may be further substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiments, R₂ and R₃ of compound of formula I and/or I(a)-I(e) are joined to form ring B as described above. In some embodiments, R₂ and R₃ are joined to form a substituted or unsubstituted, saturated or unsaturated, aliphatic (e.g. cyclopentyl, cyclohexyl), or aromatic (e.g. pyridine), carbocyclic or heterocyclic 3-8 membered ring (e.g. pyrrolidine). In some embodiments, R₂ and R₃ are joined to form an unsubstituted, saturated, aliphatic ring. In some embodiments, R₂ and R₃ are joined to form a cyclopentyl. In some embodiments, R₂ and R₃ are joined to form a cyclohexyl. In some embodiments, R₂ and R₃ are joined to form a heteroaromatic ring. In some embodiments, R₂ and R₃ are joined to form a pyridine. In some embodiments, R₂ and R₃ are joined to form a heterocyclic 3-8 membered ring. In some embodiments, R₂ and R₃ are joined to form a pyrrolidine. In some embodiments, R₂ and R₃ are joined to form a C₃-C₈ substituted or unsubstituted, aliphatic, carbocyclic or heterocyclic ring. In some embodiments, R₂ and R₃ are joined to form a C₄-C₈ substituted aliphatic ring. In some embodiments, R₂ and R₃ are joined to form a C₃-C₈ substituted aliphatic ring. In some embodiments, R₂ and R₃ are joined to form a 4-8 membered substituted or unsubstituted heterocyclic ring. In some embodiments, R₂ and R₃ are joined to form a 3-8 membered substituted or unsubstituted heterocyclic ring.

In some embodiments, R₄ of compound of formula I and/or I(a)-I(e) is H. In some embodiments, R₄ is substituted or unsubstituted C₁-C₁₀ linear or branched, or C₃-C₈ cyclic alkyl. In some embodiments, R₄ is unsubstituted C₁-C₁₀ linear or branched alkyl. In some embodiments, R₄ is substituted C₁-C₁₀ linear or branched alkyl. In some embodiments, R₄ is substituted or unsubstituted C₃-C₅ cyclic alkyl. In some embodiments, R₄ is methyl, ethyl, propyl, iso-propyl, butyl, t-Bu, iso-butyl, pentyl, benzyl; each represents a separate embodiment according to this invention. In some embodiments, R₄ is R₈—N(R₁₀)(R₁₁). In some embodiments, R₄ is CH₂—NH₂. In some embodiments, R₄ is F, Cl, Br, I, OH, SH, R₈—OH, CH₂—OH, R₈—SH, —R₈—O—R₁₀, —CH₂—O—CH₃, R₈—(C₃-C₈ cycloalkyl), R₈-(3-8 membered heterocyclic ring), CF₃, CN, NO₂, NH₂, NHR, N(R)₂, R₈—N(R₁₀)(R₁₁), C(O)H, C₁-C₅ linear or branched C(O)-haloalkyl, C(O)—CF₃, —C(O)NH₂, C(O)NHR, C(O)N(R₁₀)(R₁₁), C₂-C₅ linear or branched, substituted or unsubstituted alkenyl, CH═C(Ph)₂, C₁-C₅ linear or branched or C₃-C₈ cyclic haloalkyl, substituted or unsubstituted 3-8 membered heterocyclic ring, substituted or unsubstituted aryl, phenyl or substituted or unsubstituted benzyl; each represents a separate embodiment according to this invention. In some embodiments, R₄ is H, substituted or unsubstituted C₁-C₁₀ linear or branched, or C₃-C₅ cyclic alkyl. In some embodiments, R₄ is R₈—N(R₁₀)(R₁₁). In some embodiments, R₄ is CH₂—NH₂. In some embodiments, R₄ may be further substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiments, R₃ and R₄ of compound of formula I and/or I(a)-I(e) are joined to form a substituted or unsubstituted, saturated or unsaturated, aliphatic or aromatic, carbocyclic or heterocyclic 3-8 membered ring. In some embodiments, R₃ and R₄ are joined to form a substituted or unsubstituted, saturated aliphatic ring. In some embodiments, R₃ and R₄ are joined to form a cyclopropyl ring. In some embodiments, R₃ and R₄ are joined to form a substituted or unsubstituted, unsaturated aliphatic ring. In some embodiments, R₃ and R₄ are joined to form a substituted or unsubstituted aromatic ring. In some embodiments, R₃ and R₄ are joined to form a substituted or unsubstituted heterocyclic ring. In some embodiments, R₃ and R₄ are joined to form a ═N—OH. In some embodiments, R₃ and R₄ are joined to form a C═O.

In some embodiments, R₅ of compound of formula I, I(a), and I(b) is H. In some embodiments, R₅ is OH. In some embodiments, R₅ is SH. In some embodiments, R₅ is NH₂. In some embodiments, R₅ is NHNH₂. In some embodiments, R₅ is NHR. In some embodiments, R₅ is N(R₁₀)(R₁₁). In some embodiments, R₅ is N(H)CH₃. In some embodiments, R₅ is N(H)CH₂CH₃. In some embodiments, R₅ is R₈—N(R₁₀)(R₁₁). In some embodiments, R₅ is CH₂—NH₂. In some embodiments, R₂ and R % are joined to form a substituted or unsubstituted 4-8 membered heterocyclic ring. In some embodiments, R₂ and R₅ are joined to form a pyrrolidine. In some embodiments, R₅ is OH, NH₂, N(R₁₀)(R₁₁); each represents a separate embodiment according to this invention. In some embodiments, R₅ is N(H)CH₃ or N(H)CH₂CH₃. In some embodiments, R₅ is R₈—N(R₁₀)(R₁₁). In some embodiments, R₅ is CH₂—NH₂.

In some embodiments, R₆ of compound of formula I and I(a)-I(e) is H. In some embodiments, R₆ is F. In some embodiments, R₆ is Cl. In some embodiments, R₆ is Br. In some embodiments, R₆ is I. In some embodiments, R₆ is CN. In some embodiments, R₆ is C₁-C₅ linear or branched, substituted or unsubstituted alkyl. In some embodiments, R₆ is C₁-C₅ linear or branched, unsubstituted alkyl. In some embodiments, R₆ is C₁-C₅ linear or branched, substituted alkyl. In some embodiments, R₆ is methyl, ethyl, propyl, iso-propyl, t-Bu, iso-butyl, pentyl, benzyl; each represents a separate embodiment according to this invention. In some embodiments, R₆ is C₂-C₅ linear or branched, substituted or unsubstituted alkenyl. In some embodiments, R₆ is C₂-C₅ linear or branched, unsubstituted alkenyl. In some embodiments, R₆ is C₂-C₅ linear or branched, substituted alkenyl. In some embodiments, R₆ is ethenyl. In some embodiments, R₆ is C₁-C₅ linear or branched or C₃-C₈ cyclic haloalkyl. In some embodiments, R₆ is CF₃. In some embodiments, R₆ is C₁-C₅ linear or branched or C₃-C₈ cyclic alkoxy. In some embodiments, R₆ is C₂-C₅ linear or branched, substituted or unsubstituted alkynyl. In some embodiments, R₆ is CC—CH₂-cyclobutyl. In some embodiments, R₆ is CC—CF₃. In some embodiments, R₆ is substituted or unsubstituted C₁-C₅ linear or branched alkoxy. In some embodiments, R₆ is methoxy, ethoxy, propoxy, isopropoxy; each represents a separate embodiment according to this invention. In some embodiments, R₆ is methoxy. In some embodiments, R₆ is O—CH₂—CF₃. In some embodiments, R₆ is C₁-C₅ linear or branched or C₃-C₈ cyclic alkoxy wherein at least one methylene group (CH₂) in the alkoxy is replaced with an oxygen atom. In some embodiments, R₆ is a substituted or unsubstituted 3-8 membered heterocyclic ring. In some embodiments, R₆ is thiophene, oxazole, oxadiazole, imidazole, furane, triazole, tetrazole, pyridine, pyrimidine or pyrazine; each represents a separate embodiment according to this invention. In some embodiments, R₆ is OH, SH, R₈—OH, CH₂—OH, R₈—SH, —R₈—O—R₁₀, —CH₂—O—CH₃, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHC(O)CH₃, NHC(O)—N(R₁₀)(R₁₁), NHC(O)N(CH₃)₂, COOH, —C(O)Ph, C(O)O—R₁₀, C(O)O—CH₃, R₈—C(O)—R₁₀, CH₂C(O)CH₃, C(O)H, C(O)—R₁₀, C(O)—CH₃, C(O)—CH₂CH₃, C(O)—CH₂CH₂CH₃, C₁-C₅ linear or branched C(O)-haloalkyl, C(O)—CF₃, —C(O)NH₂, C(O)NHR, C(O)N(R₁₀)(R₁₁), C(O)N(CH₃)₂, SO₂R, SO₂N(R₁₀)(R₁₁), SO₂N(CH₃)₂, SO₂NHC(O)CH₃, substituted or unsubstituted C₃-C₈ cycloalkyl, cyclopropyl, cyclopentyl, C₂-C₅ linear or branched, substituted or unsubstituted alkenyl, ethenyl, C₂-C₅ linear or branched, substituted or unsubstituted alkynyl, ethynyl, CC—CH₂-cyclobutyl, CC—CF₃, C₁-C₅ linear or branched or C₃-C₈ cyclic haloalkyl, CF₃, CF₂CH₃, CH₂CF₃, CF₂CH₂CH₃, CH₂CH₂CF₃, CF₂CH(CH₃)₂, CF(CH₃)—CH(CH₃)₂, substituted or unsubstituted C₁-C₅ linear or branched or C₃-C₈ cyclic alkoxy, methoxy, ethoxy, propoxy, isopropoxy, O—CH₂—CF₃, C₁-C₅ linear or branched thioalkoxy, C₁-C₅ linear or branched haloalkoxy, OCF₃, OCHF₂, substituted or unsubstituted 3-8 membered heterocyclic ring, thiophene, oxazole, oxadiazole, imidazole, furane, triazole, tetrazole, pyridine, pyrimidine, pyrazine, substituted or unsubstituted aryl, phenyl or substituted or unsubstituted benzyl; each represents a separate embodiment according to this invention. In some embodiments, R₆ is H, F, Cl, Br, I, CF₃, CN, C₁-C₅ linear or branched, substituted or unsubstituted alkyl, C₂-C₅ linear or branched, substituted or unsubstituted alkenyl, C₂-C₅ linear or branched, substituted or unsubstituted alkynyl, C₁-C₅ linear or branched or C₃-C₈ cyclic haloalkyl, substituted or unsubstituted C₁-C₅ linear or branched or C₃-C₈ cyclic alkoxy, or substituted or unsubstituted 3-8 membered heterocyclic ring; each represents a separate embodiment according to this invention. In some embodiments, the alkyl is methyl or ethyl. In some embodiments, the alkenyl is ethenyl. In some embodiments, the C₁-C₅ linear or branched or C₃-C₈ cyclic haloalkyl is CF₃. In some embodiments, the alkoxy is methoxy or O—CH₂—CF₃. In some embodiments, the alkynyl is ethynyl, CC—CH₂-cyclobutyl, or CC—CF₃. In some embodiments, the substituted or unsubstituted 3-8 membered heterocyclic ring is pyridine. In some embodiments, R₆ may be further substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiments, X₁ of compound of formula I, I(a), and I(b) is 0. In some embodiments, X₁ is S. In some embodiments, X₁ is NH. In some embodiments, X₁ is NR. In some embodiments, X₁ is N—CH₃. In some embodiments, X₁ is S═O. In some embodiments, X₁ is SO₂. In some embodiments, X₁ and R₄ are joined to form a 4-8 membered heterocyclic ring. In some embodiments, X₁ and R₄ are joined to form a pyrrolidine ring. In some embodiments, X₁ is O, S, NH, or N—CH₃. In some embodiments, X₁ and R₄ are joined to form a C₄-C₅ heterocyclic ring. In some embodiments, X₁ and R₄ are joined to form a pyrrolidine.

In some embodiments, X₂ of formula I, and/or I(a)-I(c) is C. In other embodiments, X₂ is N.

In some embodiments, X₃ of formula I, and/or I(a)-I(c) is C. In other embodiments, X₃ is N.

In some embodiments, X₄ of formula I, and/or I(a)-I(c) is C. In other embodiments, X₄ is N.

In some embodiments, X₅ of formula I, and/or I(a)-I(c) is C. In other embodiments, X₅ is N.

In some embodiments, at least one of X₂-X₅ of formula I, and/or I(a)-I(c) is N. In some embodiments, at least two of X₂-X₅ are N. In some embodiments, X₂ and X₄ are N. In some embodiments, X₂ and X₅ are N. In some embodiments, X₂ and X₃ are N. In some embodiments, X₃ and X₅ are N.

In some embodiments, X₆ of formula I, and/or I(a)-I(e) is 0. In other embodiments X₆ is NH. In other embodiments X₆ is NR. In some embodiments, X₆ is CH₂. In some embodiments, X₆ is O, NH or NR.

In some embodiments, G=X of formula I, and/or I(a)-I(e) is C═O. In other embodiments G=X is SO₂. In other embodiments G(═X)—X₆—R₁ is a tetrazole moiety.

In some embodiment, R₆₀ of compound of formula I and/or I(a)-I(e) is absent. In some embodiments R₆₀ is H. In some embodiments R₆₀ is F. In some embodiments R₁₀ is Cl. In some embodiments R₁₀ is Br. In some embodiments R₆₀ is I. In some embodiments R₆₀ is CF₃. In some embodiments R₆₀ is CN. In some embodiments R₁₀ is C₁-C₅ linear or branched, substituted or unsubstituted alkyl. In some embodiments R₁₀ is C₁-C₅ linear or branched, unsubstituted alkyl. In some embodiments R₁₀ is methyl, ethyl, propyl, iso-propyl, t-Bu, iso-butyl, pentyl or benzyl; each represents a separate embodiment according to this invention. In some embodiments R₆₀ is alkenyl. In some embodiments R₆₀ is ethenyl. In some embodiments R₆₀ is C₂-C₅ linear or branched, substituted or unsubstituted alkynyl. In some embodiments R₆₀ is CC—CH₂-cyclobutyl. In some embodiments R₆₀ is CC—CF₃. In some embodiments R₆₀ is C₁-C₅ linear or branched or C₃-C₈ cyclic haloalkyl. In some embodiments R₆₀ is CF₃. In some embodiments R₆₀ is substituted or unsubstituted C₁-C₅ linear or branched or C₃-C₈ cyclic alkoxy. In some embodiments R₆₀ is methoxy. In some embodiments R₆₀ is 0-CH₂—CF₃. In some embodiments R₆₀ is substituted or unsubstituted 3-8 membered heterocyclic ring. In some embodiments R₆₀ is pyridine. In some embodiments R₆₀ is H, Cl or F. In some embodiments R₆₀ is OH, SH, R₈—OH, CH₂—OH, R₈—SH, —R₈—O—R₁₀, —CH₂—O—CH₃, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHC(O)CH₃), NHC(O)—N(R₁₀)(R₁₁), NHC(O)N(CH₃)₂, COOH, —C(O)Ph, C(O)O—R₁₀, C(O)O—CH₃, R₈—C(O)—R₁₀, CH₂C(O)CH₃, C(O)H, C(O)—R₁₀, C(O)—CH₃, C(O)—CH₂CH₃, C(O)—CH₂CH₂CH₃, C₁-C₅ linear or branched C(O)-haloalkyl, C(O)—CF₃, —C(O)NH₂, C(O)NHR, C(O)N(R₁₀)(R₁₁), C(O)N(CH₃)₂, SO₂R, SO₂N(R₁₀)(R₁₁), SO₂N(CH₃)₂, SO₂NHC(O)CH₃, C₁-C₅ linear or branched, substituted or unsubstituted alkyl, methyl, ethyl, propyl, iso-propyl, t-Bu, iso-butyl, pentyl, benzyl, substituted or unsubstituted C₃-C₈ cycloalkyl, cyclopropyl, cyclopentyl, C₂-C₅ linear or branched, substituted or unsubstituted alkenyl, ethenyl, C₂-C₅ linear or branched, substituted or unsubstituted alkynyl, ethynyl, CC—CH₂-cyclobutyl, CC—CF₃, C₁-C₅ linear or branched or C₃-C₈ cyclic haloalkyl, CF₃, CF₂CH₃, CH₂CF₃, CF₂CH₂CH₃, CH₂CH₂CF₃, CF₂CH(CH₃)₂, CF(CH₃)—CH(CH₃)₂, substituted or unsubstituted C₁-C₅ linear or branched or C₃-C₈ cyclic alkoxy, methoxy, ethoxy, propoxy, isopropoxy, O—CH₂—CF₃, C₁-C₅ linear or branched thioalkoxy, C₁-C₅ linear or branched haloalkoxy, OCF₃, OCHF₂, substituted or unsubstituted 3-8 membered heterocyclic ring, thiophene, oxazole, oxadiazole, imidazole, furane, triazole, tetrazole, pyridine, pyrimidine, pyrazine, substituted or unsubstituted aryl, phenyl, or substituted or unsubstituted benzyl; each is a separate embodiment according to this invention. In some embodiments, R₆₀ may be further substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention. In some embodiments, at least one of R₆ and R₆₀ of compound of formula I, I(a)-I(e) is not H. In some embodiments, R₆₀ is not H. In some embodiments, both R₆ and R₆₀ are not H.

In some embodiment, R₇₀ of compound of formula I(a) is absent. In some embodiments R₇₀ is H. In some embodiments R₇₀ is F. In some embodiments R₇₀ is Cl. In some embodiments R₇₀ is Br. In some embodiments R₇₀ is I. In some embodiments R₇₀ is CF₃. In some embodiments R₇₀ is CN. In some embodiments R₇₀ is C₁-C₅ linear or branched, substituted or unsubstituted alkyl. In some embodiments R₇₀ is C₁-C₅ linear or branched, unsubstituted alkyl. In some embodiments R₇₀ is methyl, ethyl, propyl, iso-propyl, t-Bu, iso-butyl, pentyl or benzyl; each represents a separate embodiment according to this invention. In some embodiments R₇₀ is alkenyl. In some embodiments R₇₀ is ethenyl. In some embodiments R₇₀ is C₂-C₅ linear or branched, substituted or unsubstituted alkynyl. In some embodiments R₇₀ is CC—CH₂-cyclobutyl. In some embodiments R₇₀ is CC—CF₃. In some embodiments R₇₀ is C₁-C₅ linear or branched or C₃-C₈ cyclic haloalkyl. In some embodiments R₇₀ is CF₃. In some embodiments R₇₀ is substituted or unsubstituted C₁-C₅ linear or branched or C₃-C₈ cyclic alkoxy. In some embodiments R₇₀ is methoxy. In some embodiments R₇₀ is O—CH₂—CF₃. In some embodiments R₇₀ is substituted or unsubstituted 3-8 membered heterocyclic ring. In some embodiments R₇₀ is pyridine. In some embodiments R₇₀ is OH, SH, R₈—OH, CH₂—OH, R₈—SH, —R₈—O—R₁₀, —CH₂—O—CH₃, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHC(O)CH₃), NHC(O)—N(R₁₀)(R₁₁), NHC(O)N(CH₃)₂, COOH, —C(O)Ph, C(O)O—R₁₀, C(O)O—CH₃, R₈—C(O)—R₁₀, CH₂C(O)CH₃, C(O)H, C(O)—R₁₀, C(O)—CH₃, C(O)—CH₂CH₃, C(O)—CH₂CH₂CH₃, C₁-C₅ linear or branched C(O)-haloalkyl, C(O)—CF₃, —C(O)NH₂, C(O)NHR, C(O)N(R₁₀)(R₁₁), C(O)N(CH₃)₂, SO₂R, SO₂N(R₁₀)(R₁₁), SO₂N(CH₃)₂, SO₂NHC(O)CH₃, C₁-C₅ linear or branched, substituted or unsubstituted alkyl, methyl, ethyl, propyl, iso-propyl, t-Bu, iso-butyl, pentyl, benzyl, substituted or unsubstituted C₃-C₅ cycloalkyl, cyclopropyl, cyclopentyl, C₂-C₅ linear or branched, substituted or unsubstituted alkenyl, ethenyl, C₂-C₅ linear or branched, substituted or unsubstituted alkynyl, ethynyl, CC—CH₂-cyclobutyl, CC—CF₃, C₁-C₅ linear or branched or C₃-C₈ cyclic haloalkyl, CF₃, CF₂CH₃, CH₂CF₃, CF₂CH₂CH₃, CH₂CH₂CF₃, CF₂CH(CH₃)₂, CF(CH₃)—CH(CH₃)₂, substituted or unsubstituted C₁-C₅ linear or branched or C₃-C₈ cyclic alkoxy, methoxy, ethoxy, propoxy, isopropoxy, O—CH₂—CF₃, C₁-C₅ linear or branched thioalkoxy, C₁-C₅ linear or branched haloalkoxy, OCF₃, OCHF₂, substituted or unsubstituted 3-8 membered heterocyclic ring, thiophene, oxazole, oxadiazole, imidazole, furane, triazole, tetrazole, pyridine, pyrimidine, pyrazine, substituted or unsubstituted aryl, phenyl, or substituted or unsubstituted benzyl; each is a separate embodiment according to this invention. In some embodiments, R₇₀ may be further substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiment, R₈₀ of compound of formula I(a) is absent. In some embodiments R₈₀ is H. In some embodiments R₈₀ is F. In some embodiments R₈₀ is Cl. In some embodiments R₈₀ is Br. In some embodiments R₈₀ is I. In some embodiments R₈₀ is CF₃. In some embodiments R₈₀ is CN. In some embodiments R₈₀ is C₁-C₅ linear or branched, substituted or unsubstituted alkyl. In some embodiments R₈₀ is C₁-C₅ linear or branched, unsubstituted alkyl. In some embodiments R₈₀ is methyl, ethyl, propyl, iso-propyl, t-Bu, iso-butyl, pentyl or benzyl; each represents a separate embodiment according to this invention. In some embodiments R₈₀ is alkenyl. In some embodiments R₈₀ is ethenyl. In some embodiments R₈₀ is C₂-C₅ linear or branched, substituted or unsubstituted alkynyl. In some embodiments R₈₀ is CC—CH₂-cyclobutyl. In some embodiments R₈₀ is CC—CF₃. In some embodiments R₈₀ is C₁-C₅ linear or branched or C₃-C₈ cyclic haloalkyl. In some embodiments R₈₀ is CF₃. In some embodiments R₈₀ is substituted or unsubstituted C₁-C₅ linear or branched or C₃-C₈ cyclic alkoxy. In some embodiments R₈₀ is methoxy. In some embodiments R₈₀ is O—CH₂—CF₃. In some embodiments R₈₀ is substituted or unsubstituted 3-8 membered heterocyclic ring. In some embodiments R₈₀ is pyridine. In some embodiments R₈₀ is OH, SH, R₈—OH, CH₂—OH, R₈—SH, —R₈—O—R₁₀, —CH₂—O—CH₃, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHC(O)CH₃), NHC(O)—N(R₁₀)(R₁₁), NHC(O)N(CH₃)₂, COOH, —C(O)Ph, C(O)O—R₁₀, C(O)O—CH₃, R₈—C(O)—R₁₀, CH₂C(O)CH₃, C(O)H, C(O)—R₁₀, C(O)—CH₃, C(O)—CH₂CH₃, C(O)—CH₂CH₂CH₃, C₁-C₅ linear or branched C(O)-haloalkyl, C(O)—CF₃, —C(O)NH₂, C(O)NHR, C(O)N(R₁₀)(R₁₁), C(O)N(CH₃)₂, SO₂R, SO₂N(R₁₀)(R₁₁), SO₂N(CH₃)₂, SO₂NHC(O)CH₃, C₁-C₅ linear or branched, substituted or unsubstituted alkyl, methyl, ethyl, propyl, iso-propyl, t-Bu, iso-butyl, pentyl, benzyl, substituted or unsubstituted C₃-C₅ cycloalkyl, cyclopropyl, cyclopentyl, C₂-C₅ linear or branched, substituted or unsubstituted alkenyl, ethenyl, C₂-C₅ linear or branched, substituted or unsubstituted alkynyl, ethynyl, CC—CH₂-cyclobutyl, CC—CF₃, C₁-C₅ linear or branched or C₃-C₈ cyclic haloalkyl, CF₃, CF₂CH₃, CH₂CF₃, CF₂CH₂CH₃, CH₂CH₂CF₃, CF₂CH(CH₃)₂, CF(CH₃)—CH(CH₃)₂, substituted or unsubstituted C₁-C₅ linear or branched or C₃-C₈ cyclic alkoxy, methoxy, ethoxy, propoxy, isopropoxy, O—CH₂—CF₃, C₁-C₅ linear or branched thioalkoxy, C₁-C₅ linear or branched haloalkoxy, OCF₃, OCHF₂, substituted or unsubstituted 3-8 membered heterocyclic ring, thiophene, oxazole, oxadiazole, imidazole, furane, triazole, tetrazole, pyridine, pyrimidine, pyrazine, substituted or unsubstituted aryl, phenyl, or substituted or unsubstituted benzyl; each is a separate embodiment according to this invention. In some embodiments, R₈₀ may be further substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiment, R₉₀ of compound of formula I(a) is absent. In some embodiments R₉₀ is H. In some embodiments R₉₀ is F. In some embodiments R₉₀ is Cl. In some embodiments R₉₀ is Br. In some embodiments R₉₀ is I. In some embodiments R₉₀ is CF₃. In some embodiments R₉₀ is CN. In some embodiments R₉₀ is C₁-C₅ linear or branched, substituted or unsubstituted alkyl. In some embodiments R₉₀ is C₁-C₅ linear or branched, unsubstituted alkyl. In some embodiments R₉₀ is methyl, ethyl, propyl, iso-propyl, t-Bu, iso-butyl, pentyl or benzyl; each represents a separate embodiment according to this invention. In some embodiments R₉₀ is alkenyl. In some embodiments R₉₀ is ethenyl. In some embodiments R₉₀ is C₂-C₅ linear or branched, substituted or unsubstituted alkynyl. In some embodiments R₉₀ is CC—CH₂-cyclobutyl. In some embodiments R₉₀ is CC—CF₃. In some embodiments R₉₀ is C₁-C₅ linear or branched or C₃-C₈ cyclic haloalkyl. In some embodiments R₉₀ is CF₃. In some embodiments R₉₀ is substituted or unsubstituted C₁-C₅ linear or branched or C₃-C₈ cyclic alkoxy. In some embodiments R₉₀ is methoxy. In some embodiments R₉₀ is O—CH₂—CF₃. In some embodiments R₉₀ is substituted or unsubstituted 3-8 membered heterocyclic ring. In some embodiments R₉₀ is pyridine. In some embodiments R₉₀ is OH, SH, R₈—OH, CH₂—OH, R₈—SH, —R₈—O—R₁₀, —CH₂—O—CH₃, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHC(O)CH₃), NHC(O)—N(R₁₀)(R₁₁), NHC(O)N(CH₃)₂, COOH, —C(O)Ph, C(O)O—R₁₀, C(O)O—CH₃, R₈—C(O)—R₁₀, CH₂C(O)CH₃, C(O)H, C(O)—R₁₀, C(O)—CH₃, C(O)—CH₂CH₃, C(O)—CH₂CH₂CH₃, C₁-C₅ linear or branched C(O)-haloalkyl, C(O)—CF₃, —C(O)NH₂, C(O)NHR, C(O)N(R₁₀)(R₁₁), C(O)N(CH₃)₂, SO₂R, SO₂N(R₁₀)(R₁₁), SO₂N(CH₃)₂, SO₂NHC(O)CH₃, C₁-C₅ linear or branched, substituted or unsubstituted alkyl, methyl, ethyl, propyl, iso-propyl, t-Bu, iso-butyl, pentyl, benzyl, substituted or unsubstituted C₃-C₅ cycloalkyl, cyclopropyl, cyclopentyl, C₂-C₅ linear or branched, substituted or unsubstituted alkenyl, ethenyl, C₂-C₅ linear or branched, substituted or unsubstituted alkynyl, ethynyl, CC—CH₂-cyclobutyl, CC—CF₃, C₁-C₅ linear or branched or C₃-C₈ cyclic haloalkyl, CF₃, CF₂CH₃, CH₂CF₃, CF₂CH₂CH₃, CH₂CH₂CF₃, CF₂CH(CH₃)₂, CF(CH₃)—CH(CH₃)₂, substituted or unsubstituted C₁-C₅ linear or branched or C₃-C₈ cyclic alkoxy, methoxy, ethoxy, propoxy, isopropoxy, O—CH₂—CF₃, C₁-C₅ linear or branched thioalkoxy, C₁-C₅ linear or branched haloalkoxy, OCF₃, OCHF₂, substituted or unsubstituted 3-8 membered heterocyclic ring, thiophene, oxazole, oxadiazole, imidazole, furane, triazole, tetrazole, pyridine, pyrimidine, pyrazine, substituted or unsubstituted aryl, phenyl, or substituted or unsubstituted benzyl; each is a separate embodiment according to this invention. In some embodiments, R₉₀ may be further substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiments, if any of X₂, X₃, X₄ and X₅ is N, then the respective substitution R₆₀, R₇₀, R₈₀ or R₉₀ is absent.

In some embodiments, R₅ of formula I and/or I(a)-I(e) is CH₂. In other embodiments, R₅ is CH₂CH₂. In other embodiments, R₅ is CH₂CH₂CH₂.

In some embodiments, p of formula I and/or I(a)-I(e) is 1. In other embodiments, p is 2. In other embodiments, p is 3.

In some embodiments, R₉ of formula I and/or I(a)-I(e) is C≡C.

In some embodiments, q of formula I and/or I(a)-I(e) is 2.

In some embodiments, R₁₀ of formula I and/or I(a)-I(e) is H. In other embodiments, R₁₀ is C₁-C₅ linear or branched alkyl. In other embodiments, R₁₀ is CH₃. In other embodiments, R₁₀ is CH₂CH₃. In other embodiments, R₁₀ is CH₂CH₂CH₃. In other embodiments, R₁₀ is CN. In other embodiments, R₁₀ is C(O)R. In other embodiments, R₁₀ is S(O)₂R. In other embodiments, R₁₀ is C(O)(OCH₃).

In some embodiments, Ru of formula I and/or I(a)-I(e) is C₁-C₅ linear or branched alkyl. In other embodiments, Ru is H. In other embodiments, Ru is CH₃. In other embodiments, Ru is CH₂CH₃. In other embodiments, Ru is CH₂CH₂CH₃. In other embodiments, Ru is CN. In other embodiments, R₁₁ is C(O)R. In other embodiments, Ru is S(O)₂R. In other embodiments, Ru is C(O)(OCH₃).

In some embodiments, R₁₀ and Ru of formula I and/or I(a)-I(e) are joined to form a substituted or unsubstituted 3-8 membered heterocyclic ring. In other embodiments, R₁₀ and Ru are joined to form a piperazine ring. In other embodiments, R₁₀ and Ru are joined to form a piperidine ring. In some embodiments, the rings may be further substituted with at least one substituent selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), COOH, alkyl ester (i.e., COOR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂; each represents a separate embodiment according to this invention.

In some embodiments, R of formula I and/or I(a)-I(e) is H. In other embodiments, R is C₁-C₅ linear or branched alkyl. In other embodiments, R is methyl. In other embodiments, R is ethyl. In other embodiments, R is C₁-C₅ linear or branched alkoxy. In other embodiments, R is methoxy. In other embodiments, R is phenyl. In other embodiments, R is aryl. In other embodiments, R is heteroaryl. In other embodiments, two gem R substituents are joined together to form a 5 or 6 membered heterocyclic ring.

In some embodiments, k is an integer number between 1 and 3. In some embodiments, k is an integer number between 1 and 2. In some embodiments, k is an integer number between 2 and 3. In some embodiments, k is 1. In some embodiments, k is 2. In some embodiments, k is 3. In some embodiments, if k is not 1, then R₂ is H. In some embodiments, if k is 2 or 3, then R₂ is H.

In some embodiments, if R₁ is H, methyl or ethyl; R₄ is H; R₃ is methyl or ethyl; and k is 1; then R₂ is not H. In some embodiments, if G=X is C═O; X₆ is O; R₁ is H, methyl or ethyl; R₄ is H; R₂ is H and R₃ is methyl or ethyl, or B ring is cyclopentyl; R₆₀ is H; and k is 1; then R₆ is not H or F. In some embodiments, if G=X is C═O; X₆ is O; R₁ is H, methyl or ethyl; R₄ is H; R₂ is H and R₃ is methyl or ethyl, or B ring is cyclopentyl; R₆₀ is H; R₆ is H or F; and k is 1; then the compound is a substantially pure single stereoisomer.

It is well understood that in structures presented in this invention wherein the carbon atom has less than 4 bonds, H atoms are present to complete the valence of the carbon. It is well understood that in structures presented in this invention wherein the nitrogen atom has less than 3 bonds, H atoms are present to complete the valence of the nitrogen.

In some embodiments, this invention is directed to the compounds listed hereinabove, agrochemical compositions and/or method of use thereof, wherein the compound is agrochemically acceptable salt, stereoisomer, optical isomer, tautomer, hydrate, N-oxide, reverse amide analog, isotopic variant (deuterated analog), or any combination thereof. In some embodiments, the compounds are herbicides. In various embodiments, the compounds are pesticides. In some embodiments, the compounds control the growth of undesired plants.

As used herein, “single or fused aromatic or heteroaromatic ring system” can be any such ring, including but not limited to phenyl, naphthyl, pyridinyl, (2-, 3-, and 4-pyridinyl), quinolinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, pyrazolyl, pyrrolyl, furanyl, thiophene-yl, quinolinyl, isoquinolinyl, 2,3-dihydroindenyl, indenyl, tetrahydronaphthyl, 3,4-dihydro-2H-benzo[b][1,4]dioxepine benzodioxolyl, benzo[d][1,3]dioxole, tetrahydronaphthyl, indolyl, 1H-indole, isoindolyl, anthracenyl, benzimidazolyl, 2,3-dihydro-1H-benzo[d]imidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, 1,2,3,4-tetrahydroquinoxaline, 1-(pyridin-1(2H)-yl)ethanone, cinnolinyl, phthalazinyl, quinolinyl, isoquinolinyl, acridinyl, benzofuranyl, 1-benzofuran, isobenzofuranyl, benzofuran-2(31)-one, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, oxadiaziolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]triazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 1H-imidazo[4,5-c]pyridine, 3H-imidazo[4,5-c]pyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, 3-methyl-4H-1,2,4-triazole, 5-methyl-1,2,4-oxadiazole, methyloxazolidin-2-one, etc.; each represents a separate embodiment according to this invention.

As used herein, the term “alkyl” can be any linear- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₈ carbons. In some embodiments, an alkyl includes C₂-C₅ carbons. In some embodiments, an alkyl includes C₂-C₈ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl), —OC(O)CF₃, —OCH₂Ph, —NHC(O)-alkyl, —C(O)Ph, C(O)O-alkyl, C(O)H, —C(O)NH₂ or any combination thereof.

The alkyl group can be a sole substituent, or it can be a component of a larger substituent, such as in an alkoxy, alkoxyalkyl, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylanido, methyl-urea, ethyl-urea, propyl-urea, 2, 3, or 4-CH₂—C₆H₄—Cl, C(OH)(CH₃)(Ph), etc.

As used herein, the term “alkenyl” can be any linear- or branched-chain alkenyl group containing up to about 30 carbons as defined hereinabove for the term “alkyl” and at least one carbon-carbon double bond. Accordingly, the term alkenyl as defined herein includes also alkadienes, alkatrienes, alkatetraenes, and so on. In some embodiments, the alkenyl group contains one carbon-carbon double bond. In some embodiments, the alkenyl group contains two, three, four, five, six, seven or eight carbon-carbon double bonds; each represents a separate embodiment according to this invention. Non limiting examples of alkenyl groups include: Ethenyl, Propenyl, Butenyl (i.e., 1-Butenyl, trans-2-Butenyl, cis-2-Butenyl, and Isobutylenyl), Pentene (i.e., 1-Pentenyl, cis-2-Pentenyl, and trans-2-Pentenyl), Hexene (e.g., 1-Hexenyl, (E)-2-Hexenyl, (Z)-2-Hexenyl, (E)-3-Hexenyl, (Z)-3-Hexenyl, 2-Methyl-1-Pentene, etc.), which may all be substituted as defined herein above for the term “alkyl”.

As used herein, the term “alkynyl” can be any linear- or branched-chain alkynyl group containing up to about 30 carbons as defined hereinabove for the term “alkyl” and at least one carbon-carbon triple bond. Accordingly, the term alkynyl as defined herein includes also alkadiynes, alkatriynes, alkatetraynes, and so on. In some embodiments, the alkynyl group contains one carbon-carbon triple bond. In some embodiments, the alkynyl group contains two, three, four, five, six, seven or eight carbon-carbon triple bonds; each represents a separate embodiment according to this invention. Non limiting examples of alkynyl groups include: acetylenyl, Propynyl, Butynyl (i.e., 1-Butynyl, 2-Butynyl, and Isobutylynyl), Pentyne (i.e., 1-Pentynyl, 2-Pentynyl), Hexyne (e.g., 1-Hexynyl, 2-Hexynyl, 3-Hexynyl, etc.), which may all be substituted as defined herein above for the term “alkyl”.

As used herein, the term “aryl” refers to any aromatic ring that is directly bound to another group and can be either substituted or unsubstituted. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, indolyl, phenylmethyl, phenylethyl, phenylamino, phenylamido, 3-methyl-4H-1,2,4-triazolyl, 5-methyl-1,2,4-oxadiazolyl, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, phenyl, halophenyl, (benzyloxy)phenyl, CN, NO₂, —CH₂CN, NH₂, NH-alkyl, N(alkyl)₂, hydroxyl, —OC(O)CF₃, —OCH₂Ph, —NHC(O)-alkyl, COOH, —C(O)Ph, C(O)O— alkyl, C(O)H, —C(O)NH₂ or any combination thereof.

As used herein, the term “alkoxy” refers to an ether group substituted by an alkyl group as defined above. Alkoxy refers both to linear and to branched alkoxy groups. Nonlimiting examples of alkoxy groups are methoxy, ethoxy, propoxy, iso-propoxy, tert-butoxy.

A “haloalkyl” group refers, in some embodiments, to an alkyl group as defined above, which is substituted by one or more halogen atoms, e.g. by F, Cl, Br or I. The term “haloalkyl” include but is not limited to fluoroalkyl, i.e., to an alkyl group bearing at least one fluorine atom. Nonlimiting examples of haloalkyl groups are CF₃, CF₂CF₃, CF₂CH₃, CH₂CF₃, CF₂CH₂CH₃, CH₂CH₂CF₃, CF₂CH(CH₃)₂ and CF(CH₃)—CH(CH₃)₂.

A “halophenyl” group refers, in some embodiments, to a phenyl substitutent which is substituted by one or more halogen atoms, e.g. by F, Cl, Br or I. In one embodiment, the halophenyl is 4-chlorophenyl.

An “alkoxyalkyl” group refers, in some embodiments, to an alkyl group as defined above, which is substituted by alkoxy group as defined above, e.g. by methoxy, ethoxy, propoxy, i-propoxy, t-butoxy etc. Nonlimiting examples of alkoxyalkyl groups are —CH₂—O—CH₃, —CH₂—O—CH(CH₃)₂, —CH₂—O—C(CH₃)₃, —CH₂—CH₂—O—CH₃, —CH₂—CH₂—O—CH(CH₃)₂, —CH₂—CH₂—O—C(CH₃)₃.

A “cycloalkyl” “cyclic alkyl” or “carbocyclic” group refers, in various embodiments, to a ring structure comprising carbon atoms as ring atoms, which may be either saturated or unsaturated, substituted or unsubstituted, single or fused. In some embodiments the cycloalkyl is a 3-10 membered ring. In some embodiments the cycloalkyl is a 3-12 membered ring. In some embodiments the cycloalkyl is a 6 membered ring. In some embodiments the cycloalkyl is a 5-7 membered ring. In some embodiments the cycloalkyl is a 3-8 membered ring. In some embodiments, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHC(O)-alkyl, —C(O)Ph, C(O)O-alkyl, C(O)H, —C(O)NH₂ or any combination thereof. In some embodiments, the cycloalkyl ring may be fused to another saturated or unsaturated cycloalkyl or heterocyclic 3-8 membered ring. In some embodiments, the cycloalkyl ring is a saturated ring. In some embodiments, the cycloalkyl ring is an unsaturated ring. Non limiting examples of a cycloalkyl group comprise cyclohexyl, cyclohexenyl, cyclopropyl, cyclopropenyl, cyclopentyl, cyclopentenyl, cyclobutyl, cyclobutenyl, cyclooctyl, cyclooctadienyl (COD), cyclooctane (COE) etc.

A “heterocycle” or “heterocyclic” group refers, in various embodiments, to a ring structure comprising in addition to carbon atoms, sulfur, oxygen, nitrogen or any combination thereof, as part of the ring. A “heteroaromatic ring” refers in various embodiments, to an aromatic ring structure comprising in addition to carbon atoms, sulfur, oxygen, nitrogen, selenium, or any combination thereof, as part of the ring. In some embodiments the heterocycle or heteroaromatic ring is a 3-10 membered ring. In some embodiments the heterocycle or heteroaromatic ring is a 3-12 membered ring. In some embodiments the heterocycle or heteroaromatic ring is a 6 membered ring. In some embodiments the heterocycle or heteroaromatic ring is a 5-7 membered ring. In some embodiments the heterocycle or heteroaromatic ring is a 3-8 membered ring. In some embodiments, the heterocycle group or heteroaromatic ring may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thiol, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHC(O)-alkyl, —C(O)Ph, C(O)O-alkyl, C(O)H, —C(O)NH₂ or any combination thereof. In some embodiments, the heterocycle ring or heteroaromatic ring may be fused to another saturated or unsaturated cycloalkyl or heterocyclic 3-8 membered ring. In some embodiments, the heterocyclic ring is a saturated ring. In some embodiments, the heterocyclic ring is an unsaturated ring. Non limiting examples of a heterocyclic ring or heteroaromatic ring systems comprise pyridine, piperidine, morpholine, piperazine, thiophene, pyrrole, benzodioxole, benzofuran-2(3H)-one, benzo[d][1,3]dioxole, indole, oxazole, isoxazole, imidazole and 1-methylimidazole, furane, triazole, pyrimidine, pyrazine, oxacyclobutane (1 or 2-oxacyclobutane), naphthalene, tetrahydrothiophene 1,1-dioxide, thiazole, benzimidazole, piperidine, 1-methylpiperidine, isoquinoline, 1,3-dihydroisobenzofuran, benzofuran, 3-methyl-4H-1,2,4-triazole, 5-methyl-1,2,4-oxadiazole, oxazolidin-2-one, methyloxazolidin-2-one or indole; each is a separate embodiment according to this invention.

In various embodiments, this invention provides a compound of this invention or its agrochemically acceptable salt, zwitterion (inner salt), stereoisomer, optical isomer, tautomer, hydrate, N-oxide, reverse amide analog, isotopic variants (e.g., deuterated analog), or any combination thereof. In various embodiments, this invention provides a single stereoisomer of the compound of this invention. In some embodiments, this invention provides an optical isomer of the compound of this invention. In some embodiments, this invention provides an agrochemically acceptable salt of the compound of this invention. In some embodiments, this invention provides a tautomer of the compound of this invention. In some embodiments, this invention provides a hydrate of the compound of this invention. In some embodiments, this invention provides an N-oxide of the compound of this invention. In some embodiments, this invention provides a reverse amide analog of the compound of this invention. In some embodiments, this invention provides an isotopic variant (including but not limited to deuterated analog) of the compound of this invention. In some embodiments, this invention provides a polymorph of the compound of this invention. In some embodiments, this invention provides a crystal of the compound of this invention. In some embodiments, this invention provides an agrochemical composition comprising a compound of this invention, as described herein, or, in some embodiments, any combination of a stereoisomer, optical isomer, agrochemically acceptable salt, tautomer, hydrate, N-oxide, isotopic variant (deuterated analog), polymorph, or crystal of the compound of this invention.

In various embodiments, the term “isomer” includes, but is not limited to, stereoisomers including optical isomers and analogs, structural isomers and analogs, conformational isomers and analogs, and the like. In some embodiments, the isomer is a stereoisomer. In another embodiment, the isomer is an optical isomer.

In various embodiments, this invention encompasses the use of various stereoisomers of the compounds of the invention. It will be appreciated by those skilled in the art that the compounds of the present invention may contain at least one chiral center. Accordingly, the compounds used in the methods of the present invention may exist in, and be isolated in, optically-active or racemic forms. The compounds according to this invention may further exist as stereoisomers which may be also optically-active isomers (e.g., enantiomers such as (R) or (S)), as enantiomerically enriched mixtures, racemic mixtures, or as single diastereomers, diastereomeric mixtures, or any other stereoisomers, including but not limited to: (R)(R), (R)(S), (S)(S), (S)(R), (R)(R)(R), (R)(R)(S), (R)(S)(R), (S)(R)(R), (R)(S)(S), (S)(R)(S), (S)(S)(R) or (S)(S)(S) stereoisomers. Some compounds may also exhibit polymorphism. It is to be understood that the present invention encompasses any racemic, optically-active, polymorphic, or stereoisomeric form, or mixtures thereof, which form possesses properties useful in controlling the growth of various undesired plants as described herein.

It is well known in the art how to prepare optically-active forms (for example, by resolution of the racemic form by recrystallization techniques, by synthesis from optically-active starting materials, by chiral synthesis, or by chromatographic separation using a chiral stationary phase).

The compounds of the present invention can also be present in the form of a racemic mixture, containing substantially equivalent amounts of stereoisomers. In some embodiments, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

In various embodiments, a compound according to the present invention comprises a substantially pure stereoisomer. In various embodiments, the compound is a mixture of stereoisomers. In various embodiments, the compound is a racemate. In various embodiments, the stereoisomer is an enantiomer. In various embodiments, the compound is a substantially pure single enantiomer. By substantially pure, it is intended that a stereoisomer is at least about 80% pure, more preferably at least about 90% pure, even more preferably at least about 95% pure, even more preferably at least about 98% pure, most preferably at least about 99% pure. In various embodiments, the compound comprises a single enantiomer in a purity of >80%; >85%; >90%; >91%; >92%; >93%; >94%; >95%; >96%; >97%; >98%; >99%; >99.5% enantiomeric excess (ee); each represents a separate embodiment according to this invention. In various embodiments, the compound comprises a single enantiomer in a purity >80%; >85%; >90%; >91%; >92%; >93%; >94%; >95%; >96%; >97%; >98%; >99%; >99.5% enantiomeric ratio (er); each represents a separate embodiment according to this invention. In various embodiments, the compound comprises a single stereoisomer in a purity higher than 80%; 85%; 90%; 91%; 92%; 93%; 94%; 95%; 96%; 97%; 98%; 99%; 99.5%; each represents a separate embodiment according to this invention.

In various embodiments, the compound is a substantially pure single enantiomer. In various embodiments, the compound is the substantially pure R enantiomer. In various embodiments, the compound is the substantially pure S enantiomer. In various embodiments, the compound comprises a mixture of enantiomers. In various embodiments, the compound is a racemate. In various embodiments, the compound comprises a mixture of stereoisomers.

In various embodiments, the compound has two chiral centers. In various embodiments, the compound comprises a mixture of stereoisomers. In various embodiments, the compound comprises a mixture of 2, 3, or 4 stereoisomers; each represents a separate embodiment according to this invention. In various embodiments, the compound is a single stereoisomer. In various embodiments, the compound is a substantially pure single stereoisomer. In various embodiments, the compound is the substantially pure RR stereoisomer. In various embodiments, the compound is the substantially pure SS stereoisomer. In various embodiments, the compound is the substantially pure RS stereoisomer. In various embodiments, the compound is the substantially pure SR stereoisomer.

Compounds of the present invention can also be in the form of a hydrate, which means that the compound further includes a stoichiometric or non-stoichiometric amount of water bound by non-covalent intermolecular forces.

As used herein, when some chemical functional group (e.g. alkyl or aryl) is said to be “substituted”, it is herein defined that one or more substitutions are possible. In some embodiments, these substitutions include but not limited to at least one selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), alkyl ester (i.e., COOR or OC(O)—R), OH, alkoxy, N(R)₂, NH₂, CF₃, aryl, phenyl, heteroaryl, C₃-C₈ cycloalkyl, halophenyl, CN and NO₂.

Compounds of the present invention may exist in the form of one or more of the possible tautomers and depending on the conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included:

### Tautomerization of the Imidazole Ring

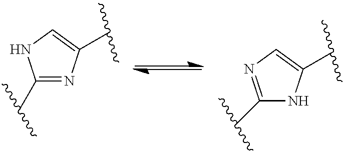 [SMILES: *c1c[nH]c(*)n1 ; *c1cnc(*)[nH]1]

### Tautomerization of the Pyrazolone Ring:

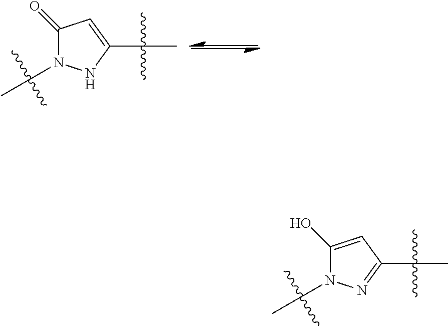 [SMILES: *c1cc(=O)n(*)[nH]1 ; *c1cc(O)n(*)n1]

The invention includes “agrochemically acceptable salts” of the compounds of this invention, which may be produced, by reaction of a compound of this invention with an acid or base. Certain compounds, particularly those possessing acidic or basic groups, can also be in the form of a salt, preferably an agrochemically acceptable salt. The term “agrochemically acceptable salt” refers to those salts that retain the agrochemical effectiveness and properties of the free bases or free acids, which are not agrochemically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable agrochemically-acceptable salts of amines of the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphates, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

### Agrochemical Composition

Another aspect of the present invention relates to an agrochemical composition including an agrochemically acceptable carrier or diluent and a compound according to the aspects of the present invention. The agrochemical composition can contain one or more of the above-identified compounds of the present invention. Typically, the agrochemical composition of the present invention will include a compound of the present invention or its agrochemically acceptable salt, as well as an agrochemically acceptable carrier or diluent. The term “agrochemically acceptable carrier” refers to any suitable adjuvants, carriers, excipients, or stabilizers, and can be in solid or liquid form such as sprays, aerosols, powders, solutions, suspensions, or emulsions.

The compounds according to the invention can be used as pesticidal or herbicidal agents in unmodified form, but they are generally formulated into compositions in various ways using formulation adjuvants, such as carriers, solvents, and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known. Such formulations can either be used directly or diluted prior to use. The dilutions can be made, for example, with water, liquid fertilizers, micronutrients, biological organisms, oil or solvents.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art.

The formulations can be prepared e.g., by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances, or combinations thereof.

The active ingredients can also be contained in very fine microcapsules. Microcapsules contain the active ingredients in a porous carrier. This enables the active ingredients to be released into the environment in controlled amounts (e.g., slow release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, dimethylformamide (DMF), dimethyl sulfoxide (DMSO), 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; as well as further substances known to the skilled in the arts.

Further adjuvants that can be used in pesticidal and/or herbicidal formulations include crystallization inhibitors, viscosity modifiers, suspending agents, dyes, anti-oxidants, foaming agents, light absorbers, mixing auxiliaries, antifoams, complexing agents, neutralizing or pH-modifying substances and buffers, corrosion inhibitors, fragrances, wetting agents, take-up enhancers, micronutrients, plasticizers, glidants, lubricants, dispersants, thickeners, antifreezes, microbicides, and liquid and solid fertilizers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Other oil derivatives are known to the skilled in the arts, for examples from the Compendium of Herbicide Adjuvants, 10^{h} Edition, Southern Illinois University, 2010.

The pesticidal and/or herbicidal compositions generally comprise from 0.1 to 99% by weight, especially from 0.1 to 95% by weight, compounds according to this invention and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha. Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 90 to 99.9%, preferably 99 to 99.9%

### Suspension Concentrates:

active ingredient: 5 to 75%, preferably 10 to 50%
water: 24 to 94%, preferably 30 to 88%
surface-active agent: 1 to 40%, preferably 2 to 30%

### Wettable Powders:

active ingredient: 0.5 to 90%, preferably 1 to 80%
surface-active agent 0.5 to 20%, preferably 1 to 15%
solid carrier: 5 to 95%, preferably 15 to 90%

### Granules:

active ingredient: 0.1 to 30%, preferably 0.1 to 15%
solid carrier: 70 to 99.5%, preferably 85 to 97%

When the compounds or agrochemical compositions of the present invention are administered to control the growth of undesired plants, the agrochemical composition can also contain, or can be administered in conjunction with, other agrochemical agents or treatment regimen presently known or hereafter developed for the growth control of various types of plants.

Accordingly, the composition of the present invention may further comprise at least one additional pesticide including but not limited to herbicide. For example, the compounds according to the invention can also be used in combination with other pesticides, herbicides, or plant growth regulators. In a preferred embodiment the additional pesticide is an herbicide and/or herbicide safener.

Examples of herbicides that can be used in combination with the compounds of the invention, include but are not limited to: acetochlor, acifluorfen (including acifluorfen-sodium), aclonifen, alachlor, alloxydim, ametryn, amicarbazone, amidosulfuron, aminocyclopyrachlor, aminopyralid, amitrole, asulam, atrazine, bensulfuron (including bensulfuron-methyl), bentazone, bicyclopyrone, bilanafos, bifenox, bispyribac-sodium, bixlozone, bromacil, bromoxynil, butachlor, butafenacil, cafenstrole, carfentrazone (including carfentrazone-ethyl); cloransulam (including cloransulam-methyl), chlorimuron (including chlorimuron-ethyl), chlorotoluron, cinosulfuron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethythexyl ester thereof), 2,4-DB, daimuron, desmedipham, dicamba (including the aluminum, aminopropyl, bis-aminopropylmethyl, choline, dichloroprop, diglycolamine, dimethylamine, dimethylammonium, potassium and sodium salts thereof), diclofop-methyl, diclosulam, diflufenican, difenzoquat, diflufenican, diflufenzopyr, dimethachlor, dimethenamid-P, diquat dibromide, diuron, esprocarb, ethalfluralin, ethofumesate, fenoxaprop (including fenoxaprop-P-ethyl), fenoxasulfone, fenquinotrione, fentrazamide, flazasulfuron, florasulam, florpyrauxifen, fluazifop (including fluazifop-P-butyl), flucarbazone (including flucarbazone-sodium); flufenacet, flumetralin, flumetsulam, flumioxazin, flupyrsulfuron (including flupyrsulfuron-methyl-sodium); fluroxypyr (including fluroxypyr-meptyl); fluthiacet-methyl, fomesafen, foramsulfuron, glufosinate (including the ammonium salt thereof), glyphosate (including the diammonium, isopropylammonium and potassium salts thereof), halauxifen (including halauxifen-methyl), halosulfuron-methyl, haloxyfop (including haloxyfop-methyl), hexazinone, hydantocidin, imazamox, imazapic, imazapyr, imazaquin, imazethapyr, indaziflam, iodosulfuron (including iodosulfuron-methyl-sodium), iofensulfuron, iofensulfuron-sodium, ioxynil, ipfencarbazone, isoproturon, isoxaben, isoxaflutole, lactofen, lancotrione, linuron, MCPA, MCPB, mecoprop-P, mefenacet, mesosulfuron, mesosulfuron-methyl, mesotrione, metamitron, metazachlor, methiozolin, metobromuron, metolachlor, metosulam, metoxuron, metribuzin, metsulfuron, molinate, napropamide, nicosulfuron, norflurazon, orthosulfamuron, oxadiargyl, oxadiazon, oxasulfuron, oxyfluorfen, paraquat dichloride, pendimethalin, penoxsulam, phenmedipham, picloram, picolinafen, pinoxaden, pretilachlor, primisulfuron-methyl, prodiamine, prometryn, propachlor, propanil, propaquizafop, propham, propyrisulfuron, propyzamide, prosulfocarb, prosulfuron, pyraclonil, pyraflufen (including pyraflufen-ethyl), pyrasulfotole, pyrazolynate, pyrazosulfuron-ethyl, pyribenzoxim, pyridate, pyriftalid, pyrimisulfan, pyrithiobac-sodium, pyroxasulfone, pyroxsulam, quinclorac, quinmerac, quizalofop (including quizalofop-P-ethyl and quizalofop-P-tefuryl), rimsulfuron, saflufenacil, sethoxydim, simazine, S-metolachlor, sulcotrione, sulfentrazone, sulfosulfuron, tebuthiuron, tefuryltrione, tembotrione, terbuthylazine, terbutryn, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tritosulfuron, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 3-[2-(3,4-dimethoxyphenyl)-6-methyl-3-oxo-pyridazine-4-carbonyl]bicyclo[3.2.1]octane-2,4-dione, 2-[2-(3,4-dimethoxyphenyl)-6-methyl-3-oxo-pyridazine-4-carbonyl]-5-methyl-cyclohexane-1,3-dione, 2-[2-(3,4-dimethoxyphenyl)-6-methyl-3-oxo-pyridazine-4-carbonyl]cyclohexane-1,3-dione, 2-[2-(3,4-dimethoxyphenyl)-6-methyl-3-oxo-pyridazine-4-carbonyl]-5,5-dimethyl-cyclohexane-1,3-dione, 6-[2-(3,4-dimethoxyphenyl)-6-methyl-3-oxo-pyridazine-4-carbonyl]-2,2,4,4-tetramethyl-cyclohexane-1,3,5-trione, 2-[2-(3,4-dimethoxyphenyl)-6-methyl-3-oxo-pyridazine-4-carbonyl]-5-ethyl-cyclohexane-1,3-dione, 2-[2-(3,4-dimethoxyphenyl)-6-methyl-3-oxo-pyridazine-4-carbonyl]-4,4,6,6-tetramethyl-cyclohexane-1,3-dione, 2-[6-cyclopropyl-2-(3,4-dimethoxyphenyl)-3-oxo-pyridazine-4-carbonyl]-5-methyl-cyclohexane-1,3-dione, 3-[6-cyclopropyl-2-(3,4-dimethoxyphenyl)-3-oxo-pyridazine-4-carbonyl]bicyclo[3.2.1]octane-2,4-dione, 2-[6-cyclopropyl-2-(3,4-dimethoxyphenyl)-3-oxo-pyridazine-4-carbonyl]-5,5-dimethyl-cyclohexane-1,3-dione, 6-[6-cyclopropyl-2-(3,4-dimethoxyphenyl)-3-oxo-pyridazine-4-carbonyl]-2,2,4,4-tetramethyl-cyclohexane-1,3,5-trione, 2-[6-cyclopropyl-2-(3,4-dimethoxyphenyl)-3-oxo-pyridazine-4-carbonyl]cyclohexane-1,3-dione, 4-[2-(3,4-dimethoxyphenyl)-6-methyl-3-oxo-pyridazine-4-carbonyl]-2,2,6,6-tetramethyl-tetrahydropyran-3,5-dione and I+4-[6-cyclopropyl-2-(3,4-dimethoxyphenyl)-3-oxo-pyridazine-4-carbonyl]-2,2,6,6-tetramethyl-tetrahydropyran-3,5-dione. These additional agents may also be present in the form of esters or salts thereof.

Compounds of the present invention may also be combined with herbicide safeners. Example of herbicide safeners include but are not limited to: benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen, N-(2-methoxybenzoyl)-4-[(methylaminocarbonyl)amino] benzenesulfonamide and oxabetrinil; all of which may be in the form of esters or salts thereof.

The compound of the invention can also be used in mixtures with other agrochemicals such as fungicides, nematicides or insecticides, examples of which are known to the skilled in the art.

The mixing ratio of compound of the invention and the additional agent, is preferably from 1:100 to 1000:1. Preferably the mixing ratio of compound of the invention to safener is from 100:1 to 1:10, especially from 20:1 to 1:1.

The mixtures can advantageously be used in the above-mentioned formulations (in which case “active ingredient” relates to the respective mixture of compound of the invention with the additional agent.

### Herbicidal Activity

In various embodiments, the invention provides compounds and compositions, including any embodiment described herein, for use in any of the methods of this invention. In various embodiments, use of a compound of this invention or a composition comprising the same, will have utility in inhibiting, suppressing, enhancing, or stimulating a desired response, as will be understood by one skilled in the art. In some embodiments, the compositions may further comprise additional active ingredients, whose activity is useful for the particular application for which the compound of this invention is being administered.

The compounds of this invention are useful as pesticides and/or herbicides. The present invention therefore further comprises a method for controlling the growth of undesired plants, comprising applying to the plants or a locus comprising them, an effective amount of a compound according to this invention, or an agrochemical composition thereof, under conditions effective to control the growth of the undesired plants, in particular the growth of weeds, in crops of useful plants.

In some embodiments, “Controlling” according to this invention refers to killing, reducing or retarding growth or preventing or reducing germination. Generally, the plants to be controlled are unwanted plants (weeds).

In some embodiments, “Locus” refers to the area in which the plants are growing or will grow.

The rates of application of compounds of the invention may vary within wide limits and depend on the nature of the soil, the method of application (for example: pre-plant, pre-emergence; post-emergence; application to the seed furrow; no tillage application etc.), the crop plant, the weed(s) to be controlled, the prevailing climatic conditions, and other factors governed by the method of application, the time of application and the target crop. The compounds of the invention are generally applied at a rate of from 10 to 2000 g/ha, especially from 50 to 1000 g/ha.

In some embodiments, the application is made by spraying the composition, typically by tractor mounted sprayer for large areas, but other methods such as dusting (for powders), drip or drench can also be used.

In some embodiments, useful plants in which the composition according to the invention can be used upon include crops such as cereals including but not limited to barley and wheat, cotton, oilseed rape, sunflower, maize, rice, soybeans, sugar beet, sugar cane and turf.

In some embodiments, crop plants also include trees, such as fruit trees, palm trees, coconut trees or other nuts. Also included are vines such as grapes, fruit bushes, fruit plants and vegetables.

In some embodiments, the crops are resistant crops. Therefore, according to some embodiments, crops also include those crops which have been rendered tolerant to herbicides or classes of herbicides (including but not limited to ALS-, GS-, EPSPS-, PPO-, ACCase- and HPPD-inhibitors) by conventional methods of breeding or by genetic engineering. Examples of crops that have been rendered tolerant to herbicides by genetic engineering methods include but not limited to glyphosate- and glufosinate-resistant maize varieties commercially available under the trade names RoundupReady® and LibertyLink®. According to other embodiments, crops also include those which have been rendered resistant to harmful insects by genetic engineering methods, examples of such crops include but are not limited to: Bt maize (resistant to European corn borer), Bt cotton (resistant to cotton boll weevil) and Bt potatoes (resistant to Colorado beetle). Non limiting examples of Bt maize include the Bt 176 maize hybrids of NK® (Syngenta Seeds). Non limiting examples of transgenic plants comprising one or more genes that code for an insecticidal resistance and express one or more toxins are: KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events).

In some embodiments, crops include those which are obtained by conventional methods of breeding or genetic engineering and contain so-called output traits (e.g., improved storage stability, higher nutritional value and improved flavor). Other useful plants include turf grass for example in golf-courses, lawns, parks and roadsides, or grown commercially for sod, and ornamental plants such as flowers or bushes.

Herbicidal compounds, or chemically active herbicides, may be broken down into pre-plant herbicides, pre-emergence herbicides and post-emergence herbicides. Pre-plant and pre-emergence herbicides typically interfere with germination of weed seeds and are applied before and after planting or sowing, respectively, but before seed germination, whereas post-emergence herbicides kill the weeds after the weed seeds have germinated and weed growth has begun.

When administering the compounds of the present invention, they can be administered in pre-plant or pre-emergence treatments, in post-emergence treatments, or both.

In various embodiments, this invention is directed to a method of controlling the growth of undesired plants, comprising applying a compound according to this invention, or an agrochemical composition thereof, to crop fields. In some embodiments, the compound is a pre-plant herbicide. In some embodiments, the compound is a pre-emergence herbicide. In some embodiments, the compound is a post-emergence herbicide. Therefore, in some embodiment, the compound is applied to crop fields before the undesired plants emerge (i.e., pre-emergence or pre-plant herbicide). In some embodiments, the compound is applied to crop fields after the undesired plants emerge (i.e., post-emergence herbicide).

In various embodiments, compounds according to this invention, and agrochemical compositions thereof, are used to control undesired plants, which include a wide variety of monocotyledonous and dicotyledonous weed species.

In some embodiments, the undesired plant is a weed. In some embodiments, the undesired plant is a eudicot (dicotyledonous or dicot). In some embodiments, the undesired plant is a monocotyledon (monocotyledonous or monocot).

Non limiting examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor; each represents a separate embodiment according to this invention.

Non limiting examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium; each represents a separate embodiment according to this invention.

In some embodiments, the undesired plant is Abutilon theophrasti, Amaranthus palmeri, Ambrosia artemisiifolia, Alopecurus myosuroides, Avena sterilis, Chenopodium album, Conyza Canadensis, Digitaria sanguinalis, Echinochloa colona, Euphorbia heterophylla, Lolium perenne, Lolium rigidum, Matricaria chamomilla, Phalaris paradoxa, Poa annua, Portulaca oleracea, Setaria viridis, Solanum nigrum or any combination thereof. In some embodiments, the compound is any one of the compounds listed in Table 1 and 2; each compound represents a separate embodiment according to this invention.

In some embodiments, compounds, and compositions according to this invention are utilized to control undesirable vegetation in rice. In certain embodiments, the undesirable vegetation is Brachiaria platyphylla (Groseb.) Nash (broadleaf signalgrass, BRAPP), Digitaria sanguinalis (L.) Scop, (large crabgrass, DIGSA), Echinochloa crus-galli (L.) P. Beauv. (bamyardgrass, ECHCG), Echinochloa colonum (L.) LINK (junglerice, ECHCO), Echinochloa oryzoides (Ard.) Fritsch (early watergrass, ECHOR), Echinochloa oryzicola (Vasinger) Vasinger (late watergrass, ECHPH), Ischaemum rugosum Salisb. (saramollagrass, ISCRU), Leptochloa chinensis (L.) Nees (Chinese sprangletop, LEFCH), Leptochloa fascicularis (Lam.) Gray (bearded sprangletop, LEFFA), Leptochloa panicoides (Presl.) Hitchc. (Amazon sprangletop, LEFPA), Panicum dichotomiflorum (L.) Michx. (fall panicum, PANDI), Paspalum dilatatum Poir. (dallisgrass, PASDI), Cyperus difformis L. (smallflower flatsedge, CYPDI), Cyperus esculentus L. (yellow nutsedge, CYPES), Cyperus iria L. (rice flatsedge, CYPIR), Cyperus rotundus L. (purple nutsedge, CYPRO), Eleocharis species (ELOSS), Fimbristylis miliacea (L.) Vahl (globe fringerush, FIMMI), Schoenoplectus juncoides Roxb. (Japanese bulrush, SCPJU), Schoenoplectus maritimus L. (sea clubrush, SCPMA), Schoenoplectus mucronatus L. (ricefield bulrush, SCPMU), Aeschynomene species, (jointvetch, AESSS), Alternanthera philoxeroides (Mart.) Griseb. (alligatorweed, ALRPH), Alisma plantago-aquatica L. (common waterplantain, ALSPA), Amaranthus species, (pigweeds and amaranths, AMASS), Ammannia coccinea Rottb. (redstem, AMMCO), Eclipta alba (L.) Hassk. (American false daisy, ECLAL), Heteranthera limosa (SW.) Willd./Vahl (ducksalad, HETLI), Heteranthera reniformis R. & P. (roundleaf mudplantain, HETRE), Ipomoea hederacea (L.) Jacq. (ivyleaf momingglory, IPOHE), Lindernia dubia (L.) Pennell (low false pimpernel, LIDDU), Monochoria korsakowii Regel & Maack (monochoria, MOOKA), Monochoria vaginalis (Burm. F.) C. Presl ex Kuhth, (monochoria, MOOVA), Murdannia nudifiora (L.) Brenan (doveweed, MUDNU), Polygonum pensylvanicum L. (Pennsylvania smartweed, POLPY), Polygonum persicaria L. (ladysthumb, POLPE), Polygonum hydropiperoides Michx. (POLHP, mild smartweed), Rotala indica (Willd.) Koehne (Indian toothcup, ROTIN), Sagittaria species, (arrowhead, SAGSS), Sesbania exaltata (Raf) Cory/Rydb. Ex Hill (hemp sesbania, SEBEX), or Sphenoclea zeylanica Gaertn. (gooseweed, SPDZE); each is a separate embodiment according to this invention. In some embodiments, the compound is any one of the compounds listed in Table 1 and 2; each compound represents a separate embodiment according to this invention.

In some embodiments, the compounds and compositions according to this invention are utilized to control undesirable vegetation in cereals. In certain embodiments, the undesirable vegetation is Alopecurus myosuroides Huds. (blackgrass, ALOMY), Apera spica-venti (L.) Beauv. (windgrass, APESV), Avena fatua L. (wild oat, AVEFA), Bromus tectorum L. (downy brome, BROTE), Lolium multiflorum Lam. (Italian ryegrass, LOLMU), Phalaris minor Retz. (littleseed canarygrass, PHAMI), Poa annua L. (annual bluegrass, POAAN), Setaria pumila (Poir.) Roemer & J. A. Schultes (yellow foxtail, SETLU), Setaria viridis (L.) Beauv. (green foxtail, SETVI), Cirsium arvense (L.) Scop. (Canada thistle, CIRARy), Galium aparine L. (catchweed bedstraw, GALAP), Kochia scoparia (L.) Schrad. (kochia, KCHSC), Lamium purpureum L. (purple deadnettle, LAMPU), Matricaria recutita L. (wild chamomile, MATCH), Matricaria matricarioides (Less.) Porter (pineappleweed, MATMT), Papaver rhoeas L. (common poppy, PAPRH), Polygonum convolvulus L. (wild buckwheat, POLCO), Salsola tragus L. (Russian thistle, SASKR), Stellaria media (L.) VilL (common chickweed, STEME), Veronica persica Poir. (Persian speedwell, VERPE), Viola arvensis Murr. (field violet, VIOAR), or Viola tricolor L. (wild violet, VIOTR); each is a separate embodiment according to this invention. In some embodiments, the compound is any one of the compounds listed in Table 1 and 2; each compound represents a separate embodiment according to this invention.

In some embodiments, the compounds and compositions according to this invention are utilized to control undesirable vegetation in range and pasture. In certain embodiments, the undesirable vegetation is Ambrosia artemisiifolia L. (common ragweed, AMBEL), Cassia obtusifolia (sickle pod, CASOB), Centaurea maculosa auct, non-Lam. (spotted knapweed, CENMA), Cirsium arvense (L.) Scop. (Canada thistle, CIRAR), Convolvulus arvensis L. (field bindweed, CONAR), Euphorbia esula L. (leafy spurge, EPHES), Lactuca serriola L./Tom. (prickly lettuce, LACSE), Plantago lanceolata L. (buckhom plantain, PLALA), Rumex obtusifolius L. (broadleaf dock, RUMOB), Sida spinosa L. (prickly sida, SIDSP), Sinapis arvensis L. (wild mustard, SINAR), Sonchus arvensis L. (perennial sowthistle, SONAR), Solidago species (goldenrod, SOOSS), Taraxacum officinale G. H. Weber ex Wiggers (dandelion, TAROF), Trifolium repens L. (white clover, TRFRE), or Urtica dioica L. (common nettle, URTDI); each is a separate embodiment according to this invention. In some embodiments, the compound is any one of the compounds listed in Table 1 and 2; each compound represents a separate embodiment according to this invention.

In some embodiments, the compounds and compositions according to this invention are utilized to control undesirable vegetation found in row crops. In certain embodiments, the undesirable vegetation is Alopecurus myosuroides Huds. (blackgrass, ALOMY), Avena fatua L. (wild oat, AVEFA), Brachiaria platyphylla (Groseb.) Nash (broadleaf signalgrass, BRAPP), Digitaria sanguinalis (L.) Scop, (large crabgrass, DIGSA), Echinochloa crus-galli (L.) P. Beauv. (bamyardgrass, ECHCG), Echinochloa colonum (L.) Link (junglerice, ECHCO), Lolium multiflorum Lam. (Italian ryegrass, LOLMU), Panicum dichotomiflorum Michx. (fall panicum, PANDI), Panicum miliaceum L. (wild-proso millet, PANMI), Setariafaberi Herrm. (giant foxtail, SETFA), Setaria viridis (L.) Beauv. (green foxtail, SETVI), Sorghum halepense (L.) Pers. (Johnsongrass, SORHA), Sorghum bicolor (L.) Moench ssp. Arundinaceum (shattercane, SORVU), Cyperus esculentus L. (yellow nutsedge, CYPES), Cyperus rotundus L. (purple nutsedge, CYPRO), Abutilon theophrasti Medik. (velvetleaf, ABUTH), Amaranthus species (pigweeds and amaranths, AMASS), Ambrosia artemisiifolia L. (common ragweed, AMBEL), Ambrosia psilostachya DC. (western ragweed, AMBPS), Ambrosia trifida L. (giant ragweed, AMBTR), Asclepias syriaca L. (common milkweed, ASCSY), Chenopodium album L. (common lambsquarters, CHEAL), Cirsium arvense (L.) Scop. (Canada thistle, CIRAR), Commelina benghalensis L. (tropical spiderwort, COMBE), Datura stramonium L. (jimsonweed, DATST), Daucus carota L. (wild carrot, DAUCA), Euphorbia heterophylla L. (wild poinsettia, EPHHL), Erigeron bonariensis L. (hairy fleabane, ERIBO), Erigeron canadensis L. (Canadian fleabane, ERICA), Helianthus annuus L. (common sunflower, HELAN), Jacquemontia tamnifolia (L.) Griseb. (smallflower momingglory, IAQTA), Ipomoea hederacea (L.) Jacq. (ivyleaf momingglory, IPOHE), Ipomoea lacunosa L. (white momingglory, IPOLA), Lactuca serriola L./Tom. (prickly lettuce, LACSE), Portulaca oleracea L. (common purslane, POROL), Sida spinosa L. (prickly sida, SIDSP), Sinapis arvensis L. (wild mustard, SINAR), Solanum ptychanthum Dunal (eastern black nightshade, SOLPT), or Xanthium strumarium L. (common cocklebur, XANST); each is a separate embodiment according to this invention. In some embodiments, the compound is any one of the compounds listed in Table 1 and 2; each compound represents a separate embodiment according to this invention.

The following examples are presented in order to more fully illustrate the preferred embodiments of the invention. They should in no way, however, be construed as limiting the broad scope of the invention.

### EXAMPLES

### Example 1

### Synthetic Details for Compounds of the Invention

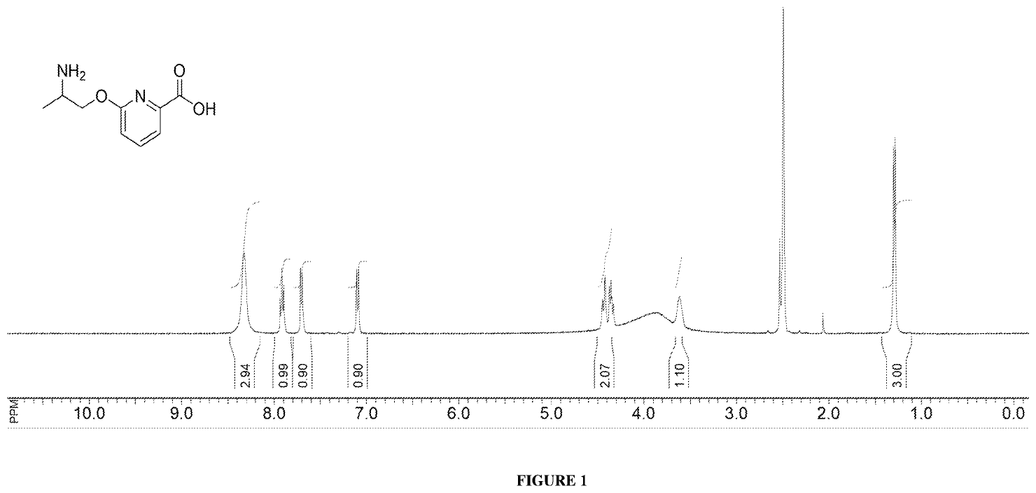

Compounds 116, 174, 175 and their analogs, are synthesized as depicted in scheme 1 below. The first step involves a Mitsunobu coupling (Mitsunobu, O., Synthesis, 1 (1981)) between an hydroxypyridine and the desired, amino-protected, aminoalcohol. This coupling is followed by two de-protection steps: basic hydrolysis of the ester and acidic cleavage of the amino group. The NMR spectrum for compound 116 is depicted in FIG. 1.

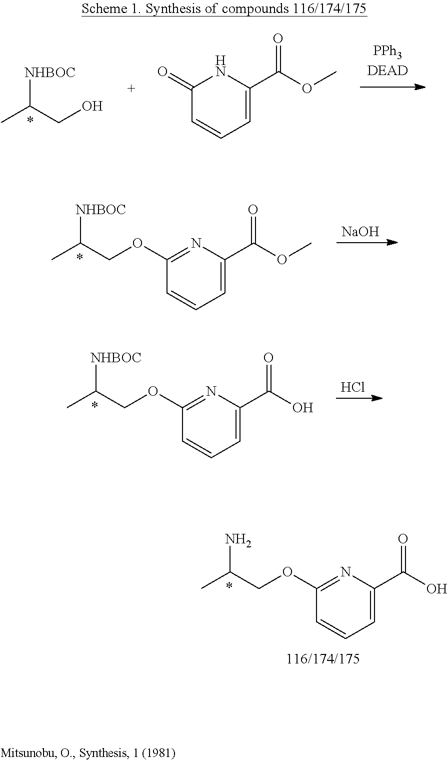

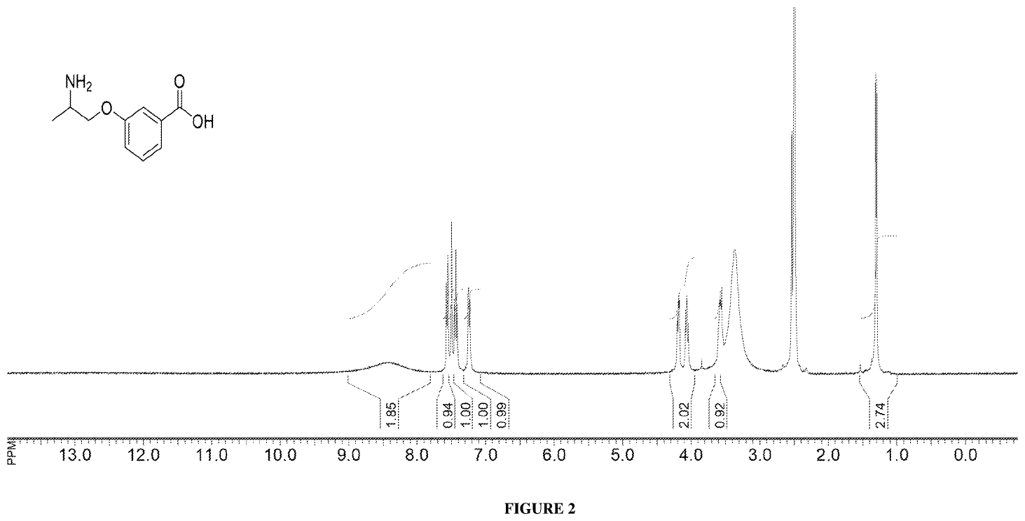

Compounds 106, 154, 155 and their analogs, are synthesized as depicted in scheme 2 below. The first step involves a phenolic nucleophilic attack on the desired, suitably protected, aminoalcohol as described in Bower et al. [Bower, John F. et al., Org Lett, 9(17), (2007)]. This reaction is followed by two de-protection steps: basic hydrolysis of the ester and acidic cleavage of the amino group. The NMR spectrum for compound 106 is depicted in FIG. 2.

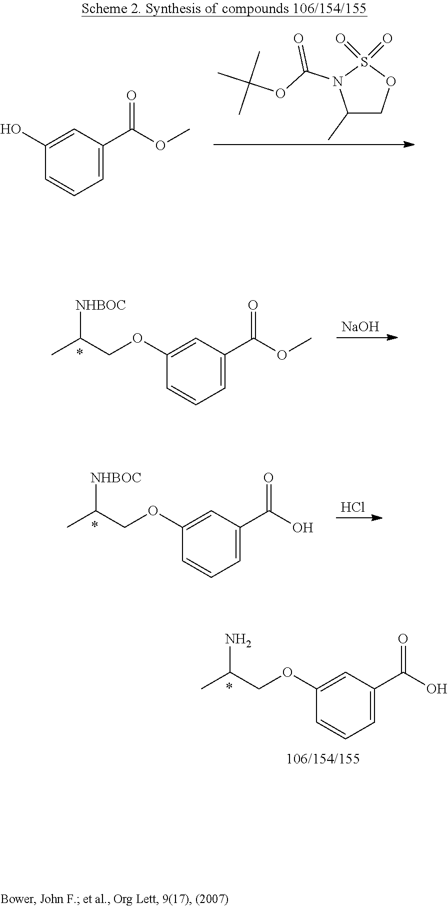

### Synthesis of Compound 167

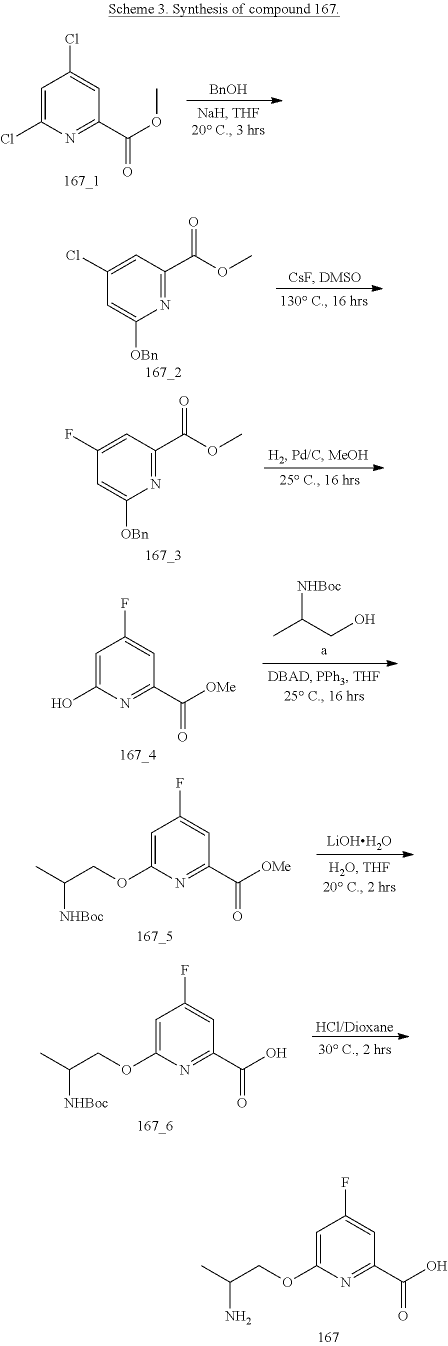

### Step 1—Synthesis of Compound 167_2

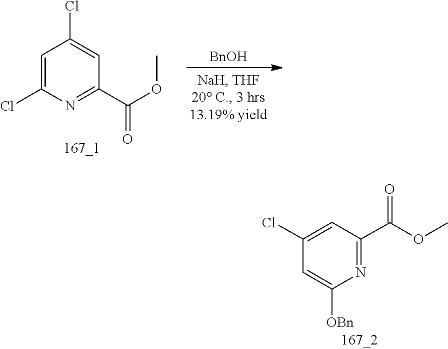 [SMILES: COC(=O)c1cc(Cl)cc(Cl)n1 ; "BnOH" ; COC(=O)c1cc(Cl)cc(OCc2ccccc2)n1]

To a solution of NaH (6.11 g, 152.84 mmol, 60% purity, 1 eq) in THF (300 mL) was added BnOH (16.53 g, 152.84 mmol, 15.89 mL, 1 eq) and compound 167_1 (31.49 g, 152.84 mmol, 1 eq) at 0° C. The reaction mixture was stirred at 20° C. for 3 hrs. LCMS showed the starting material was consumed and 48.2% of desired product was formed. The reaction mixture was poured into water (100 mL) and extracted with EA (100 mL*3). The combined organic layers were dried over MgSO₄, filtered and concentrated to give crude product. The crude was purified by column chromatography (SiO₂, Ethyl acetate:Petroleum ether=0 to 3/97) to give compound 1672 (10.0 g, 20.17 mmol, 13.19% yield, 56% purity) as a yellow solid, confirmed by LCMS (56.6%).

### Step 2—Synthesis of Compound 167_3

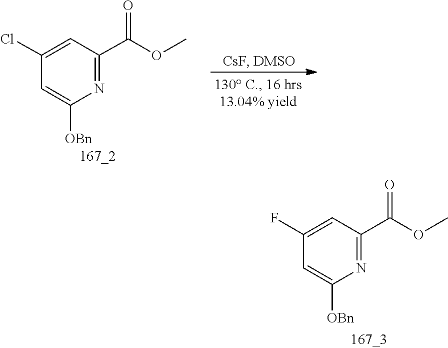

To s solution of compound 167_2 (10 g, 36.01 mmol, 1 eq) in DMSO (25 mL) was added CsF (16.41 g, 108.03 mmol, 3.98 mL, 3 eq) in one portion. The reaction mixture was stirred at 130° C. for 16 hrs. LCMS showed the starting material was consumed and 19.6% of desired product was formed. The reaction mixture was poured into water (50 mL) and filtered. The filtrate was extracted with EtOAc (50 mL*3). The combined organic layers were dried over MgSO₄, filtered and concentrated to give crude product. The crude was purified by column chromatography (SiO₂, Ethyl acetate:Petroleum ether=0 to 7/93) to give compound 1673 (3 g, 4.70 mmol, 13.04% yield, 40.9% purity) as yellow solid.

### Step 3—Synthesis of Compound 167_4

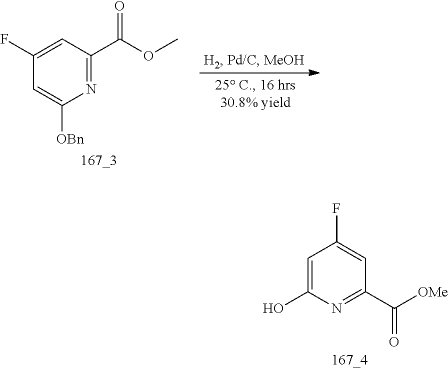

To a solution of compound 1673 (3.00 g, 11.48 mmol, 1 eq) in MeOH (20 mL) was added Pd/C (2.00 g, 1.88 mmol, 10% purity, 1.64e-1 eq) under N₂ at 25° C. The suspension was degassed under vacuum and purged with H₂ for several times. The mixture was stirred under H₂ (15 psi) at 25° C. for 16 hours. LCMS showed the starting material was consumed and 32.6% of desired product was formed. The mixture was filtered with celite and the filtrate was concentrated under reduced pressure to give a colourless oil. The crude was purified by column chromatography (SiO₂, Tetrahydrofuran/Petroleum ether=0 to 46/54) to give compound 1674 (0.682 g, 3.55 mmol, 30.89% yield, 89% purity) as a white solid, confirmed by LCMS.

### Step 4—Synthesis of Compound 167_5

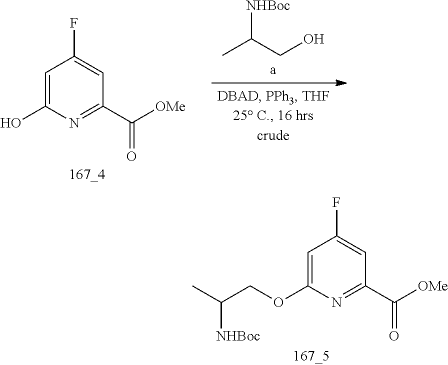 [SMILES: COC(=O)c1cc(F)cc(O)n1 ; CC(CO)NC(=O)OC(C)(C)C ; COC(=O)c1cc(F)cc(OCC(C)NC(=O)OC(C)(C)C)n1]

To a mixture of compound 1674 (680 mg, 3.97 mmol, 1 eq), compound a (1.04 g, 5.96 mmol, 1.5 eq) and PPh₃ (1.56 g, 5.96 mmol, 1.5 eq) in THF (14 mL) was added DBAD (1.37 g, 5.96 mmol, 1.5 eq) in batches at 0-10° C. After addition, the reaction mixture was stirred at 25° C. for 16 hrs. LCMS showed the starting material was consumed and 18.1% of desired product was formed. The reaction mixture was poured into water (50 mL) and extracted with EtOAc (50 mL*3), washed with brine (50 mL*2). The combined organic layers were dried over MgSO₄, filtered and concentrated to give crude product, which was purified by column chromatography (SiO₂, Ethylacetate/Petroleum ether=0 to 9/91) to give compound 167_5 (2.00 g, crude) as a white solid, confirmed by LCMS (94.76%).

### Step 5—Synthesis of Compound 167_6

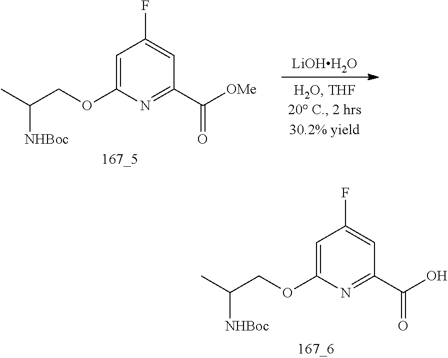 [SMILES: COC(=O)c1cc(F)cc(OCC(C)NC(=O)OC(C)(C)C)n1 ; "LiOH·H2O" ; CC(COc1cc(F)cc(C(=O)O)n1)NC(=O)OC(C)(C)C]

To a solution of compound 1675 (2 g, 6.09 mmol, 1 eq) in THF (20 mL) and H₂O (20 mL) was added LiOH·H₂O (383.39 mg, 9.14 mmol, 1.5 eq) at 20° C. The reaction was stirred at 20° C. for 2 hrs. LCMS showed the starting material was consumed and 94.4% of desired product was formed. The reaction mixture was poured into water (50 mL) and extracted with MTBE (50 mL*3). The aqueous phase was acidified with 1 M HCl to pH=4 and extracted with EtOAc (50 mL*3). The combined organic layers were dried over MgSO₄, filtered and concentrated to give compound 167_6 (0.594 g, 1.84 mmol, 30.25% yield, 97.5% purity) as a yellow solid, confirmed by LCMS (97.5%).

### Step 6—Synthesis of Compound 167

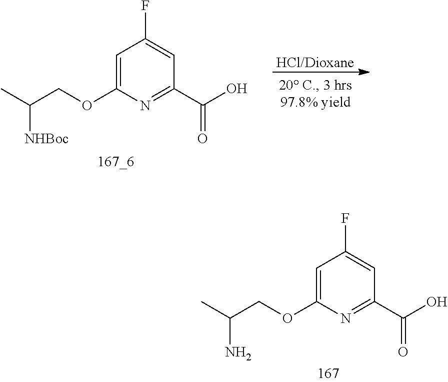 [SMILES: CC(COc1cc(F)cc(C(=O)O)n1)NC(=O)OC(C)(C)C ; "HCl/Dioxane" ; CC(N)COc1cc(F)cc(C(=O)O)n1]

To a solution of compound 1676 (0.594 g, 1.89 mmol, 1 eq) in dioxane (6 mL) was added HCl/dioxane (4 M, 6 mL, 12.70 eq) dropwise at 0-10° C. The reaction mixture was stirred at 20° C. for 3 hrs. LCMS showed the starting material was consumed and 99.0% of desired product was formed. The reaction mixture was concentrated to give Compound 167 (0.468 g, 1.85 mmol, 97.81% yield, 99.0% purity, HCl) as a white solid.

¹H NMR (400 MHz, DMSO-d) δ 8.64-8.12 (m, 3H), 7.69-7.45 (m, 1H), 7.15-6.89 (m, 1H), 4.50-4.45 (m, 1H), 4.41-4.37 (m, 1H), 3.66-3.60 (m, 1H), 1.36-1.20 (m, 3H)

### Synthesis of Compound 173

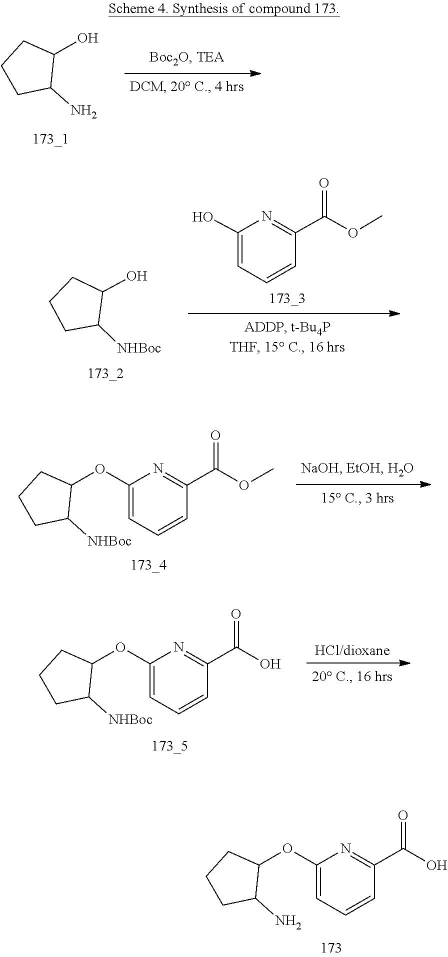 [SMILES: NC1CCCC1O ; CC(C)(C)OC(=O)NC1CCCC1O ; COC(=O)c1cccc(O)n1 ; COC(=O)c1cccc(OC2CCCC2NC(=O)OC(C)(C)C)n1 ; CC(C)(C)OC(=O)NC1CCCC1Oc1cccc(C(=O)O)n1 ; NC1CCCC1Oc1cccc(C(=O)O)n1]

### Step 1—Synthesis of Compound 173_2

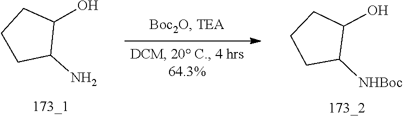 [SMILES: NC1CCCC1O ; CC(C)(C)OC(=O)NC1CCCC1O]

To a solution of compound 173_1 in DCM (50 mL) was added TEA (7.50 g, 74.2 mmol) and Boc₂O (8.09 g, 37.1 mmol). The mixture was stirred at 20° C. for 4 hrs. TLC (PE:EtOAc=1:1, SM R_{f}=0.0, Product R_{f}=0.5) indicated reactant 173_1 was consumed completely, and one major new spot with larger polarity was detected. The reaction mixture was concentrated under reduced pressure to remove THF. The residue was purified by flash silica gel chromatography (ISCO®; 40 g SepaHash® Silica Hash Column, Eluent of 0-50% Ethyl acetate/Petroleum ethergradient @ 50 mL/min). Compound 1732 (3.20 g, 64.3% yield) was obtained as a white solid.

¹H NMR: (400 MHz, CDCl₃) δ 4.70 (br s, 1H), 4.04-3.94 (m, 1H), 3.70-3.58 (m, 1H), 2.14-2.06 (m, 1H), 2.03-1.96 (m, 1H), 1.84-1.58 (m, 4H), 1.45 (s, 9H)

### Step 2—Synthesis of Compound 173_4

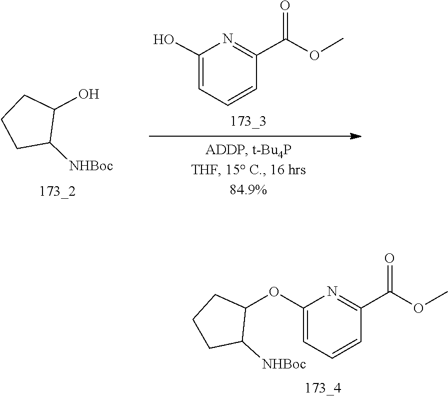 [SMILES: CC(C)(C)OC(=O)NC1CCCC1O ; COC(=O)c1cccc(O)n1 ; COC(=O)c1cccc(OC2CCCC2NC(=O)OC(C)(C)C)n1]

To a solution of compound 1733 (1.20 g, 7.83 mmol), compound 173_2 (1.50 g, 7.45 mmol) in THF (30 mL) was added ADDP (3.95 g, 15.7 mmol) and tributylphosphane (4.52 g, 22.4 mmol). The mixture was stirred at 15° C. for 16 hrs. LC-MS showed reactant 173_3 was consumed completely and desired mass was detected. The reaction mixture was concentrated under reduced pressure to remove THF. The residue was diluted with H₂O (20 mL) and extracted with EtOAc (10 mL*3). The combined organic layers were filtered and concentrated under reduced pressure to give a residue. The crude product was triturated with MTBE (25 mL) for 30 min. The product is in the filtrate. The residue was purified by flash silica gel chromatography (ISCO®; 12 g SepaHash® Silica Hash Column, Eluent of 0-20% Ethyl acetate/Petroleum ether gradient @ 20 m/min). Compound 1734 (2.13 g, 84.9% yield) was obtained as yellow oil.

¹H NMR: (400 MHz, DMSO-d) 37.87-7.82 (m, 1H), 7.63 (d, J=7.1 Hz, 1H), 7.01 (d, J=8.0 Hz, 1H), 6.71 (br d, J=7.9 Hz, 1H), 3.87-3.82 (m, 3H), 2.06-1.81 (m, 2H), 1.77-1.48 (m, 5H), 1.30-1.17 (m, 9H)

### Step 3—Synthesis of Compound 173_5

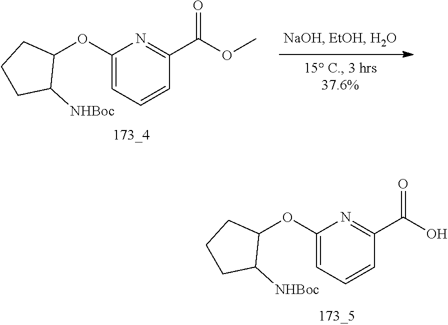 [SMILES: COC(=O)c1cccc(OC2CCCC2NC(=O)OC(C)(C)C)n1 ; CC(C)(C)OC(=O)NC1CCCC1Oc1cccc(C(=O)O)n1]

To a solution of compound 1734 (1.00 g, 2.97 mmol) in EtOH (10 mL) and H₂O (10 mL) was added NaOH (357 mg, 8.92 mmol). The mixture was stirred at 15° C. for 3 hrs. LC-MS showed reactant 173_4 was consumed completely and one main peak with desired mass was detected. The reaction mixture was acidified to 3 by addition 1M HCl (5 mL) and then extracted with EtOAc (30 mL*3). The combined organic layers were washed with brine (40 mL), dried over MgSO₄, filtered and concentrated under reduced pressure to give a residue. The residue was purified by prep-HPLC (column: Xtimate C18 150*40 mm*10 um; mobile phase: [water (0.05% HCl)-ACN]; B %: 40%-70%, 10 min). Compound 173_5 (360 mg, 37.6% yield) was obtained as a white solid.

### Step 4—Synthesis of Compound 173

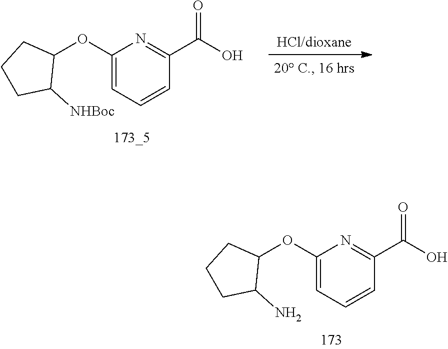 [SMILES: CC(C)(C)OC(=O)NC1CCCC1Oc1cccc(C(=O)O)n1 ; NC1CCCC1Oc1cccc(C(=O)O)n1]

To a solution of compound 1735 (326 mg, 1.01 mmol) in dioxane (3 mL) was added HCl/dioxane (3 mL). The mixture was stirred at 20° C. for 16 hrs. LC-MS showed reactant 173_5 was consumed completely and one main peak with desired mass was detected. The reaction mixture was concentrated under reduced pressure to remove solvent. The crude product was triturated with EtOAc (10 mL) for 16 hrs. CPD 173 (269 mg, crude, HCl) was obtained as a white solid.

¹H NMR: (400 MHz, DMSO-d) δ 8.32 (br s, 3H), 7.95-7.86 (m, 1H), 7.69 (d, J=7.0 Hz, 1H), 7.09 (d, J=8.1 Hz, 1H), 5.49-5.41 (m, 1H), 3.75-3.63 (m, 1H), 2.17-2.00 (m, 2H), 1.91-1.74 (m, 3H), 1.69-1.53 (m, 1H)

### Synthesis of Compound 180

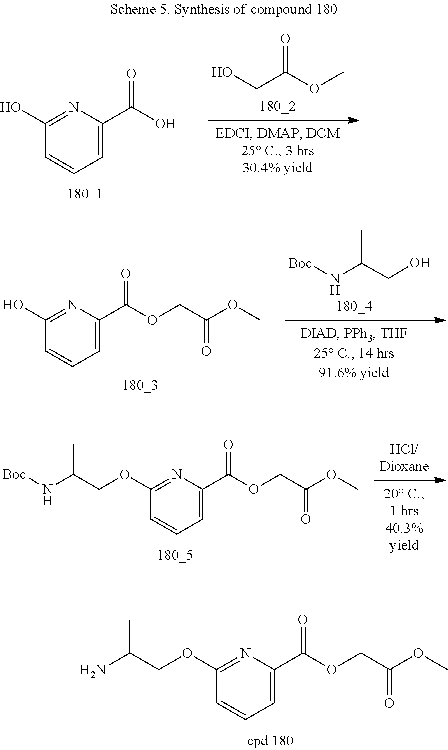

### Step 1—Synthesis of Compound 180_3

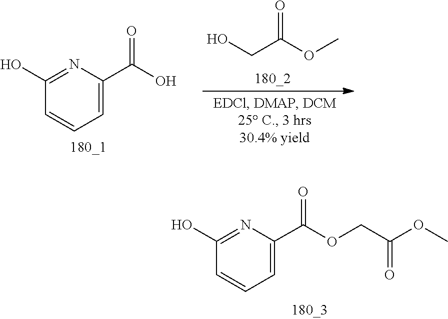

To a solution of compound 180_1 (3.00 g, 21.6 mmol) in DCM (60 mL) was added compound 180_2 (1.94 g, 21.6 mmol, 1.66 mL) and EDCI (5.37 g, 28.0 mmol), DMAP (263 mg, 2.16 mmol). The mixture was stirred at 25° C. for 3 hrs. TLC (petroleum ether:ethyl acetate=3:1, R_{f}=0.4) indicated compound 180_2 was consumed completely and one new spot with larger polarity formed. The reaction mixture was concentrated under reduced pressure to remove solvent. The residue was diluted with water (30 mL) and extracted with DCM (30 mL*3). The combined organic layers were washed with brine (90 mL), dried over MgSO₄, filtered and concentrated under reduced pressure to give a residue. The residue was purified by flash silica gel chromatography (ISCO®; 40 g SepaHash® Silica Hash Column, Eluent of 0-2% DCM/MeOH gradient @ 40 mL/min) to give compound 1803 (1.40 g, 6.56 mmol, 30.4% yield, 99% purity) as a white solid.

### Step 2—Synthesis of Compound 180_5

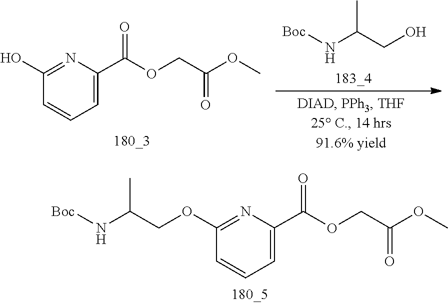 [SMILES: COC(=O)COC(=O)c1cccc(O)n1 ; CC(CO)NC(=O)OC(C)(C)C ; COC(=O)COC(=O)c1cccc(OCC(C)NC(=O)OC(C)(C)C)n1]

To a mixture of compound 1803 (1.40 g, 6.63 mmol) and compound 1834 (1.16 g, 6.63 mmol) in dry THF (40 mL) was added PPh₃ (1.74 g, 6.63 mmol), followed by the addition of DIAD (1.34 g, 6.63 mmol, 1.29 mL) at 0° C. under N₂, and then the mixture was stirred at 25° C. for 14 hrs under N₂ atmosphere. LC-MS showed compound 183_4 was consumed completely and one main peak with desired mass was detected. The reaction mixture was concentrated under reduced pressure to remove solvent. The residue was diluted with water (30 mL) and extracted with ethyl acetate (30 mL*3). The combined organic layers were washed with brine (90 mL), dried over Na₂SO₄ and concentrated under reduced pressure to give a residue. The residue was purified by flash silica gel chromatography (ISCO®; 60 g SepaHash® Silica Hash Column, Eluent of 0-19% Ethyl acetate/Petroleum ether gradient @ 40 mL/min) to give compound 180_5 (2.3 g, 6.06 mmol, 91.6% yield, 97% purity) as a white solid.

¹H NMR (400 MHz, CDCl₃) δ 7.83-7.77 (m, 1H), 7.76-7.69 (m, 1H), 7.04-6.96 (m, 1H), 5.08-4.92 (m, 4H), 4.91 (s, 2H), 4.42-4.33 (m, 2H), 4.20-4.06 (m, 1H), 3.81 (s, 3H), 1.45 (s, 9H).

### Step 3—Synthesis of Compound 180

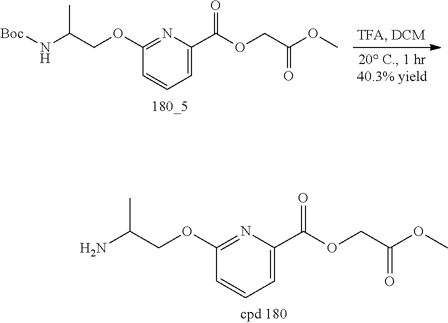 [SMILES: COC(=O)COC(=O)c1cccc(OCC(C)NC(=O)OC(C)(C)C)n1 ; COC(=O)COC(=O)c1cccc(OCC(C)N)n1]

A mixture of compound 1805 (1.00 g, 2.71 mmol) and 4 M HCl/dioxane (20 mL) was stirred at 20° C. for 1 hr. LC-MS showed compound 180_5 was consumed completely and one main peak with desired mass was detected. The reaction mixture was concentrated under reduced pressure to remove solvent. The residue was diluted with DCM (10 mL) and extracted with water (10 mL*3). The water phase was freeze-dried under vacuum to give a residue. The residue was purified by prep-HPLC [HCl condition, column: Xtimate C18 150*40 mm*10 um; mobile phase: [water (0.05% HCl)-ACN]; B %: 0%-30.0%, 10 min] to give cpd 180 (300 mg, 1.09 mmol, 40.3% yield, 97.8% purity) as a white solid.

¹H NMR (400 MHz, DMSO-d₆) δ 8.37 (br s, 3H), 8.01-7.94 (m, 1H), 7.78 (dd, J=2.2, 7.1 Hz, 1H), 7.18 (dd, J=4.1, 8.3 Hz, 1H), 4.96 (s, 1H), 4.84 (s, 2H), 4.44-4.37 (m, 2H), 3.71 (s, 2H), 3.63 (br s, 1H), 1.31 (d, J=6.8 Hz, 3H).

### Synthesis of Compound 181

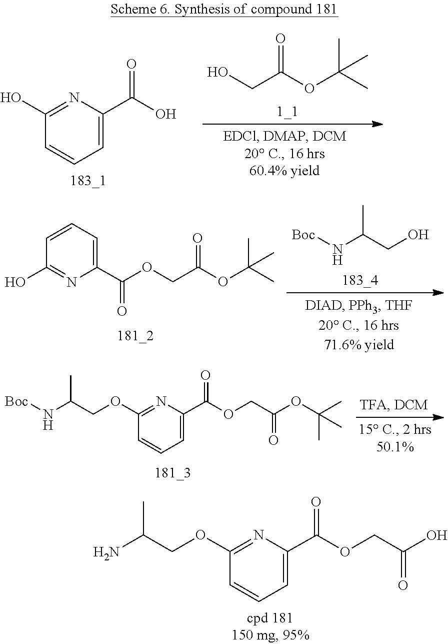

### Step 1—Synthesis of Compound 181_2

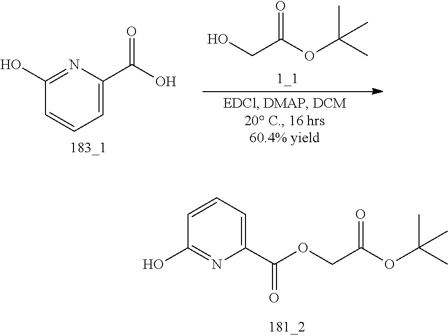

To a solution of compound 1831 (500 mg, 3.59 mmol) and compound 1_1 (475 mg, 3.59 mmol) in DCM (2 mL) was added EDCI (896 mg, 4.67 mmol) and DMAP (44.0 mg, 359 umol). The mixture was stirred at 20° C. for 16 hrs. LC-MS showed the reaction was complete and one main peak with desired mass was detected. The reaction mixture was diluted with H₂O (50 mL) and extracted with EtOAc (50 mL*3). The combined organic layers were washed with brine (100 mL), dried over MgSO₄, filtered and concentrated under reduced pressure to give a residue. The residue was purified by flash silica gel chromatography (ISCO®; 20 g SepaFlash® Silica Hash Column, Eluent of 0-20% DCM/MeOH @ 20 m/min) to give compound 181_2 (550 mg, 60.4% yield) as a white solid.

¹H NMR: (400 MHz, DMSO-d₆) δ 11.56 (br d, J=2.8 Hz, 1H), 7.71 (dd, J=7.3, 8.5 Hz, 1H), 7.30 (br d, J=6.0 Hz, 1H), 6.80 (d, J=8.8 Hz, 1H), 4.79 (s, 2H), 1.43 (s, 9H).

### Step 2—Synthesis of Compound 181_3

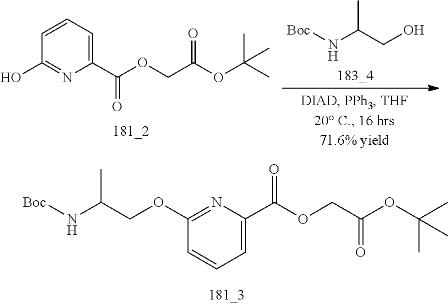

To a solution of compound 1812 (500 mg, 1.97 mmol) and compound 1834 (346 mg, 1.97 mmol) in THF (15 mL) was added DIAD (399 mg, 1.97 mmol) and PPh₃ (517 mg, 1.97 mmol). The mixture was stirred at 20° C. for 16 hrs. LC-MS showed the reaction was complete and one main peak with desired mass was detected. The reaction mixture was diluted with H₂O (50 mL) and extracted with EtOAc (50 mL*3). The combined organic layers were washed with brine (100 mL), dried over MgSO₄, filtered and concentrated under reduced pressure to give a residue. The residue was purified by flash silica gel chromatography (ISCO®; 20 g SepaHash® Silica Hash Column, Eluent of 0-20% Ethyl acetate/Petroleum ether gradient @ 20 m/min) to give compound 181_3 (580 mg, 71.6% yield) as a white solid.

¹H NMR: (400 MHz, DMSO-d₆) δ 7.92 (dd, J=7.4, 8.2 Hz, 1H), 7.73 (d, J=6.8 Hz, 1H), 7.12 (d, J=8.1 Hz, 1H), 6.92 (br d, J=8.1 Hz, 1H), 4.81 (s, 2H), 4.16 (br d, J=6.0 Hz, 2H), 3.93-3.81 (m, 1H), 1.44 (s, 9H), 1.38 (s, 9H), 1.12 (d, J=6.8 Hz, 3H).

### Step 3—Synthesis of Compound 181

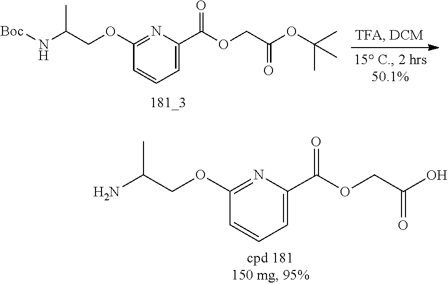 [SMILES: CC(COc1cccc(C(=O)OCC(=O)OC(C)(C)C)n1)NC(=O)OC(C)(C)C ; CC(N)COc1cccc(C(=O)OCC(=O)O)n1]

To a solution of compound 1813 (500 mg, 1.22 mmol) in DCM (5 mL) was added TFA (1 mL). The mixture was stirred at 15° C. for 2 hrs. LC-MS showed reactant was consumed completely and one main peak with desired mass was detected. The reaction mixture was filtered and concentrated under reduced pressure to give a residue. The residue was purified by prep-HPLC (HCl condition; column: Xtimate C18 150*40 mm*10 um; mobile phase: [water (0.05% HCl)-ACN]; B %: 0%-30%, 10 min), to give compound 181 (155 mg, 50.1% yield) as a white solid.

¹H NMR: (400 MHz, DMSO-d₆) δ 8.37 (br s, 3H), 7.98 (t, J=7.8 Hz, 1H), 7.79 (d, J=7.1 Hz, 1H), 7.19 (d, J=8.3 Hz, 1H), 4.85 (s, 2H), 4.45-4.32 (m, 2H), 3.64 (br s, 1H), 1.32 (br d, J=6.6 Hz, 3H)

### Synthesis of Compound 182

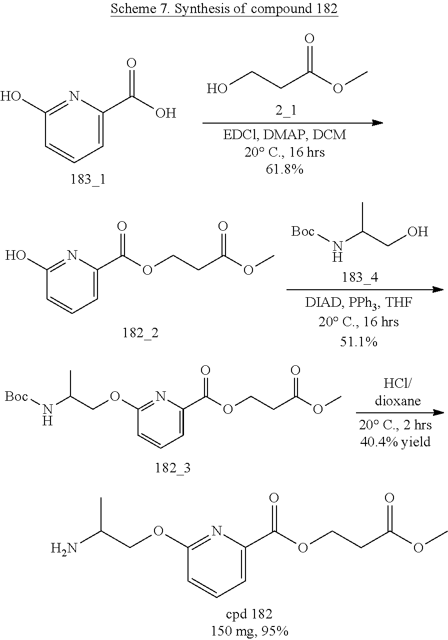

### Step 1—Synthesis of Compound 182_2

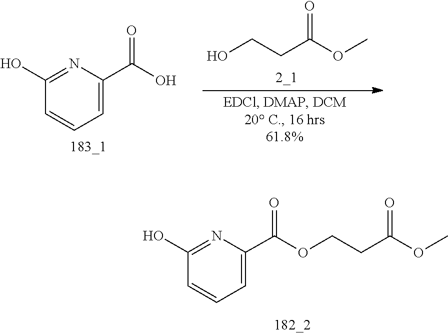

To a solution of compound 183_1 (1 g, 7.19 mmol) and compound 21 (748.5 mg, 7.19 mmol) in DCM (20 mL) was added EDCI (1.79 g, 9.35 mmol) and DMAP (87.8 mg, 719 μmol). The mixture was stirred at 20° C. for 16 hrs. LC-MS showed compound 183_1 was consumed completely and one main peak with desired mass was detected. The reaction mixture was diluted with H₂O (50 mL) and extracted with EtOAc (50 mL*3). The combined organic layers were washed with brine (100 mL), dried over MgSO₄, filtered and concentrated under reduced pressure to give a residue. The residue was purified by flash silica gel chromatography (ISCO®; 20 g SepaHash® Silica Hash Column, Eluent of 0-7% DCM/MeOH @ 20 mL/min) to give compound 1822 (1.00 g, 61.8% yield) as a white solid.

¹H NMR: (400 MHz, DMSO-d₆) δ 11.58 (br s, 1H), 7.68-7.60 (m, 1H), 7.19-7.07 (m, 1H), 6.73 (d, J=8.8 Hz, 1H), 4.47 (t, J=6.1 Hz, 2H), 3.64 (s, 4H), 2.83 (t, J=6.1 Hz, 2H).

### Step 2—Synthesis of Compound 182_3

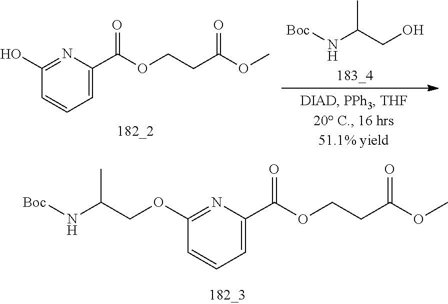 [SMILES: COC(=O)CCOC(=O)c1cccc(O)n1 ; CC(CO)NC(=O)OC(C)(C)C ; COC(=O)CCOC(=O)c1cccc(OCC(C)NC(=O)OC(C)(C)C)n1]

To a solution of compound 1822 (1.17 g, 6.66 mmol) and compound 1834 (1.00 g, 4.44 mmol) in THF (15 mL) was added DIAD (97.9 mg, 4.44 mmol) and PPh₃ (1.16 g, 4.44 mmol). The mixture was stirred at 20° C. for 16 hrs. TLC (petroleum ether:ethyl acetate=4:1, R_{f}=0.19) showed the reaction was complete. The reaction mixture was diluted with H₂O (50 mL) and extracted with EtOAc (50 mL*3). The combined organic layers were washed with brine (100 mL), dried over MgSO₄, filtered and concentrated under reduced pressure to give a residue. The residue was purified by flash silica gel chromatography (ISCO®; 80 g SepaHash® Silica Hash Column, Eluent of 0-17% Ethyl acetate/Petroleum ether gradient @ 80 mL/min) to give compound 1823 (1.30 g, 3.40 mmol, 51.1% yield) as a white solid.

¹H NMR: (400 MHz, CDCl₃) δ 7.75-7.67 (m, 2H), 7.00-6.92 (m, 1H), 4.64 (t, J=6.4 Hz, 2H), 4.46-4.29 (m, 2H), 4.10 (br s, 1H), 3.75 (s, 3H), 2.85 (t, J=6.4 Hz, 2H), 1.44 (s, 9H), 1.26 (s, 3H).

### Step 3—Synthesis of Compound 182

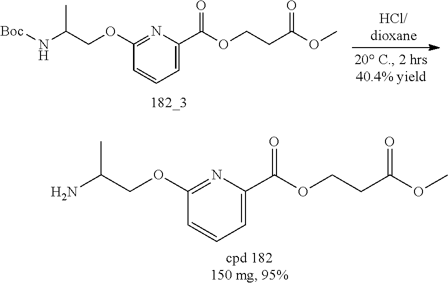 [SMILES: COC(=O)CCOC(=O)c1cccc(OCC(C)NC(=O)OC(C)(C)C)n1 ; COC(=O)CCOC(=O)c1cccc(OCC(C)N)n1]

A solution of 182_3 (0.50 g, 1.31 mmol) in 4 M HCl/dioxane (5 mL) was stirred at 20° C. for 2 hrs. LCMS showed the reaction was complete. The reaction mixture was concentrated under reduced pressure to give a residue, the residue was redisolved in water (20 mL), washed with DCM (15 mL*3), the water phase was concentrated under vacuum to give a residue. The residue was purified by prep-HPLC (HCl condition, column: Xtimate C18 150*40 mm*10 um; mobile phase: [water (0.05% HCl)-ACN]; B %: 0%-35%,10 min) to give cpd 182 (300 mg, 1.06 mmol, 40.4% yield, 99.4% purity) as a white solid.

¹H NMR: (400 MHz, DMSO-d₆) δ 8.39 (br s, 3H), 7.95 (t, J=8.0 Hz, 1H), 7.74-7.64 (m, 1H), 7.15 (d, J=8.0 Hz, 1H), 4.52-4.40 (m, 4H), 4.37-4.30 (m, 1H), 3.64 (s, 3H), 2.86-2.70 (m, 2H), 1.31 (d, J=6.6 Hz, 3H).

### Synthesis of Compound 183

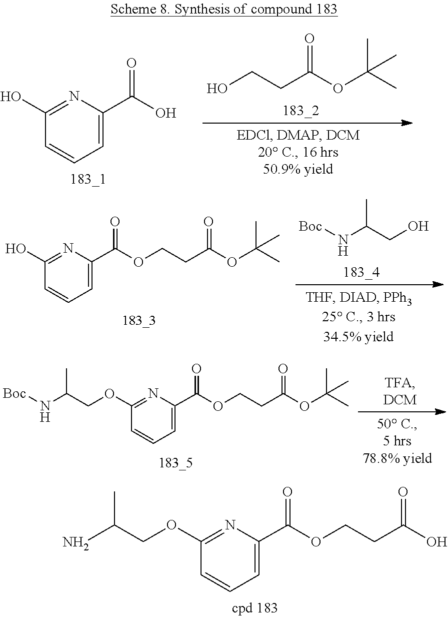

### Step 1—Synthesis of Compound 183_3

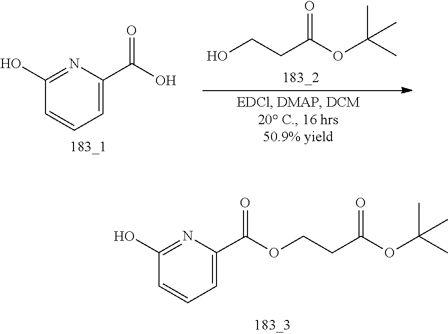

To a solution of compound 1831 (952 mg, 6.84 mmol,) in DCM (40 mL) was added compound 183_2 (1.00 g, 6.84 mmol, 1.01 mL), EDCI (1.70 g, 8.89 mmol) and DMAP (83.6 mg, 684 μmol). The mixture was stirred at 20° C. for 16 hrs. TLC (petroleum ether:ethyl acetate=10:1, R_{f}=0.3) indicated compound 183_2 was consumed completely and one new spot with larger polarity formed. The reaction was clean according to TLC. The reaction mixture was concentrated under reduced pressure to remove solvent. The residue was diluted with water (30 mL) and extracted with DCM (30 mL*3). The combined organic layers were washed with brine (90 mL), dried over MgSO₄, filtered and concentrated under reduced pressure to give a residue. The residue was purified by flash silica gel chromatography (ISCO®; 40.0 g SepaFlash® Silica Hash Column, Eluent of 0-2% MeOH/DCM @ 10.0 mL/min) to give compound 183_3 (930 mg, 3.48 mmol, 50.9% yield) as a white solid.

### Step 2—Synthesis of Compound 183_5

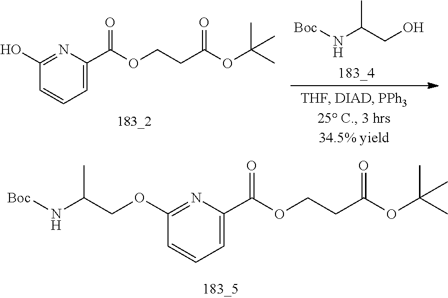

To a mixture of compound 1833 (930 mg, 3.48 mmol) and compound 1834 (670 mg, 3.83 mmol) in dry THF (10 mL) was added PPh₃ (1.00 g, 3.83 mmol), followed by the addition of DIAD (774 mg, 3.83 mmol, 744 μL) at 0° C. under N₂, and then the mixture was stirred at 25° C. for 3 hrs under N₂ atmosphere. LC-MS showed compound 183_4 was consumed completely and one main peak with desired mass was detected. The reaction mixture was concentrated under reduced pressure to remove solvent. The residue was diluted with water (30 mL) and extracted with DCM (30 mL*3). The combined organic layers was washed with brine (90 mL), dried over MgSO₄, filtered and concentrated under reduced pressure to give a residue. The residue was purified by flash silica gel chromatography (ISCO®; 20.0 g SepaHash® Silica Hash Column, Eluent of 0-17% Ethyl acetate/Petroleum ether gradient @ 20 m/min) to give compound 183_5 (510 mg, 1.20 mmol, 34.5% yield) as a white solid.

¹H NMR: (400 MHz, CDCl₃) δ 7.65-7.58 (m, 2H), 6.90-6.84 (m, 1H), 5.13-4.76 (m, 1H), 4.52 (t, J=6.5 Hz, 2H), 4.37-4.19 (m, 2H), 4.01 (br s, 1H), 2.65 (s, 2H), 1.37 (d, J=10.1 Hz, 18H), 1.18 (d, J=6.8 Hz, 3H).

### Step 3—Synthesis of Compound 183

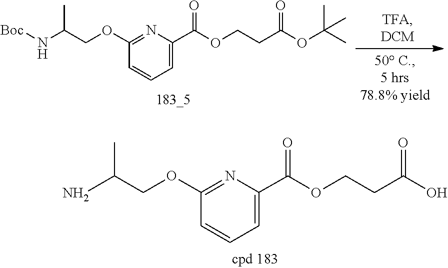 [SMILES: CC(COc1cccc(C(=O)OCCC(=O)OC(C)(C)C)n1)NC(=O)OC(C)(C)C ; CC(N)COc1cccc(C(=O)OCCC(=O)O)n1]

To a solution of compound 1835 (600 mg, 1.41 mmol) in DCM (8 mL) was added TFA (3.08 g, 27.0 mmol, 2.00 mL). The mixture was stirred at 50° C. for 5 hrs. LC-MS showed compound 183_5 was consumed completely and one main peak with desired mass was detected. The reaction mixture was concentrated under reduced pressure to remove solvent. The residue was dissolved in DCM (10 mL) and extracted with water (10 mL*3). The water phase was freeze-dried under vacuum to give a residue. The residue was purified by prep-HPLC [vcolumn: Xtimate C18 150*40 mm*10 m; mobile phase: [water (0.05% HCl)-ACN]; B %: 0%-30%, 10 min] to give cpd 183 (300 mg, 1.11 mmol, 78.8% yield, 99.6% purity) as a white solid.

¹H NMR: (400 MHz, DMSO-d₆) δ 8.35 (br s, 3H), 7.95 (dd, J=7.4, 8.2 Hz, 1H), 7.69 (d, J=7.3 Hz, 1H), 7.15 (d, J=8.3 Hz, 1H), 4.49-4.40 (m, 4H), 3.67-3.59 (m, 1H), 2.73 (t, J=6.2 Hz, 2H), 1.31 (d, J=6.6 Hz, 3H).

### Example 2

### Herbicidal Activity Data

### Applications on a Weed Panel

Herbicidal activity of compounds (active ingredient; A.I.) was demonstrated by the following greenhouse experiments:

### In-Planta Low Through-Put Screens (LTP) Results

### Post-Emergence Treatments

A basic panel of eight weed species (Table 3) sowed in 4×4×7 cm plastic pots containing a garden mix (klasmann). Each specie was sowed in a separate pot. In each pot, 10-15 seeds were sowed according to the specie viability. Timing of application determined at a 1-2 true leaf stage. The plants grew for 30 days in a controlled greenhouse (26±2° C. day, 20±2° C. night). Hood irrigation (tap water+Shefer 5:3:8 8 mM) was given at a 50% water content by weight. Two days before application, the tested plants thinned down to three plants per pot. Compounds were soluble in water (DDW), and commercial herbicide control was soluble in formulation B (Table 4). Before application, 1% (v/v) crop oil and 0.02% (v/v) surfactant (Tergitol™ 15-5-7) were added to the solution. Application was conducted with an industrial sprayer (TeeJet 6502E nozzle) at a rate of 2 kg/ha and spray volume of 4801/ha. Plants were evaluated at 3 time points (4, 8, 12 days after application (DAA)). At each time point, visual phenotyping was recorded using a scale of 0-6 (0: no visible effect, 6: maximum effect). At 12 DAA, plants foliage was harvested, dried and weighed for dry weight analysis.

Post-emergence advanced dose response experiment included 4 species: SETVI, ECHCO, AMAPA, ABUTH (Tables 3, 5). Application was conducted at six rates between 0.6-0.0187 kg/ha and spray volume of 4801/ha. Plants were evaluated at 4 time points (6, 12, 18 and 26 DAA). At each time point, visual phenotyping was recorded using a scale of 0-6 (0: no visible effect, 6: maximum effect). At 26 DAA, plants foliage was harvested, dried and weighed for dry weight analysis.

Post-emergence advanced wide panel experiment included 24 weed species (Table 5). Application was conducted at 2 rates of 2 kg/ha and 0.25 kg/ha and spray volume of 4801/ha. Visual phenotyping was recorded at 4, 11, 17 and 20 DAA using a scale of 0-6 (0: no visible effect, 6: maximum effect). At 21 DAA, plants foliage was harvested, dried, and weighed for dry weight analysis. All experiments included an untreated control, a solvent control, and a positive control (commercial herbicide A.I.). Statistical analysis for visual phenotyping determined by a median value of ≥3.5 and Fisher test (pval≤0.05). Statistical analysis for dry weight determined by % inhibition ≥50 and T test (pval≤0.05), as well as Wilcox test (pval≤0.05).

### Pre-Emergence Treatments

A basic panel of 8 weed species (Table 3) were sowed in 4×4×7 cm plastic pots containing inert sand (Sweet sand), intensively washed using osmosis water. Each specie was sowed in a separate pot. In each pot 10-15 seeds were sowed according to the specie viability. Sowing was performed one day before application. The plants were grown for 21 days in a controlled greenhouse (26±2° C. day 20±2° C. night). Hood irrigation (tap water+Shefer 5:3:8 8 mM) was given at a 50% water content by weight. Compounds were soluble in water (DDW), and commercial herbicide control was soluble in formulation B (Table 4). Application was conducted with an industrial sprayer (TeeJet 6502E nozzle) at a rate of 2 kg/ha and spray volume of 4801/ha.

Pre-emergence advanced dose response experiment application was conducted at six rates between 1-0.0312 kg/ha and spray volume of 4801/ha. Percentage of emergence was evaluated at 15 DAA. Visual phenotyping was recorded using a scale of 0-6 (0: no visible effect, 6: maximum effect) at 18 DAA. All experiments included an untreated control, a solvent control and a positive control (commercial herbicide A.I.). Statistical analysis for visual phenotyping determined by a median value of ≥3.5 and Fisher test (pval≤0.05). Statistical analysis for plant emergence determined by % emergence ≥50 and T test (pval≤0.05), as well as Wilcox test (pval≤0.05).

TABLE 3
Basic weed panel species

| Bayer code | Scientific name |
|---|---|
| ABUTH | Abutilon theophrasti |
| AMBEL | Ambrosia artemisiifolia |
| AMAPA | Amaranthus palmeri |
| MATCH | Matricaria chamomilla |
| ALOMY | Alopecurus myosuroides |
| POAAN | Poa annua |
| LOLPE | Lolium perenne |
| SETVI | Setaria viridis |

TABLE 4
Formulations

| Formulation | Ingredients |
|---|---|
| A | 100% DDW |
| B | 48.5% acetone |
| | 40% DDW |
| | 10% isopropyl alcohol |
| | 1.5% DMSO |

TABLE 5
Advanced weed panel species

| Bayer code | Scientific name |
|---|---|
| ABUTH | Abutilon theophrasti |
| AMBEL | Ambrosia artemisiifolia |
| AMAPA | Amaranthus palmeri |
| MATCH | Matricaria chamomilla |
| ERICA | Conyza Canadensis |
| EPHHL | Euphorbia heterophylla |
| AMARE | Amaranthus retroflexus |
| SOLNI | Solanum nigrum |
| CHEAL | Chenopodium album |
| POROL | Portulaca oleracea |
| GLXMA | Glycine max |
| BRSNN | Brassica napus |
| ALOMY | Alopecurus myosuroides |
| POAAN | Poa annua |
| LOLPE | Lolium perenne |
| SETVI | Setaria viridis |
| DIGSA | Digitaria sanguinalis |
| ECHCO | Echinochloa colona |
| LOLRI | Lolium rigidum |
| PHAPA | Phalaris paradoxa |
| ZEAMX | Zea mays |
| AVEST | Avena sterilis |
| TRZAX | Triticum aestivum |
| ORYSA | Oryza sativa |

### Post-Emergence Applications on Miniature Dicot Model Plants

Arabidopsis thaliana seeds were sown in 96 well plates filled with irrigated Sweet sand (10%>clay) that is washed from salts and minerals using tap water. 5-10 seeds were sown in the center of each well. 7-8 days post sowing, at 2 true leaves stage, thinning out was performed to ensure that compound application is done on a single plant per well. The plates were placed in a controlled greenhouse in a random order inside a bath which allows flooding irrigation with tap water supplemented with a fertilizer. Applied compounds were dissolved into a final solution of 50% acetone, 49.9% DDW, 0.1% tween-20.

A 96 well plate was used as a stock plate for preparing application solutions for 8 repeats. Each row contained different concentrations per chemical. Maximal concentration for application was 1.5 Kg/Ha and dilution factor is 2.5. Chemical application was performed one day after thinning out in a chemical hood. 5 μL applied on first two true leaves in each well using 12 channel pipettes. Data collection: RGB (red, green, blue) data for green area per well was documented using camera. Data is collected at a few time points during the experiment: one day after thinning and before chemical application, two-, six- or nine-days post application. During the last two documentations, visual phenotyping was performed. Data analysis: Given RGB results and visual phenotype scores, a student t-test conducted to compare between treatment and control performance for continuous data (RGB) and Fisher exact test to analyze the non-continuous data (phenotypic scores). Dose-Response curves are generated for each treatment to infer ED₅₀ and max. inhibition parameters, using treatment's log concentration range as the dependent variable and normalized green area.

### Pre-Emergence Applications on Dicot and Monocot Model Plants

These experiments are conducted similarly except that either Arabidopsis thaliana or Eragrostis teff seeds were sowed (5-10 or 5-7, respectively), plant thinning out was not performed, compound dissolved into a final solution of 50% acetone, 49.9% DDW, compound application was done at 30 μL volume per well directly on sowed soil before plant emergence and data was collected 10 or 7 days after chemical application on dicot or monocot plants, respectively.

### Imaging, RGB and Statistical Analysis

Plate imaging performed at 11 and 18 DAA. RGB data for green area per well was documented and used to extract % inhibition. Dose-Response curves were built for each treatment to infer ED₅₀, ED₇₅ and ED₉₀ parameters, using treatment's log concentration range as the independent variable and normalized green area as the dependent variable. Treatments were compared to controls performance given RGB results and using student's t-test (p-val≤0.05).

### Results:

The in-planta HTPS (high throughput on soil) and in vitro inhibitory results for selected compounds are presented in Table 6 below:

TABLE 6
Herbicidal activity (in vitro and in-planta)
for compounds according to this invention.

| | | | HTPS - Dicot - | | HTPS - Dicot - | | HTPS - Monocot - | |
|---|---|---|---|---|---|---|---|---|
| | in vitro | | Post | | Pre | | Pre | |
| | Thermal | | | Max | | Max | | Max |
| Compound | Shift | K_{d} | ED₅₀ | effect | ED₅₀ | effect | ED₅₀ | effect |
| No. | (° C.) | (mM) | (g/ha) | (%) | (g/ha) | (%) | (g/ha) | (%) |
| 108 | 16 | TBD | 31 | 100 | 31 | 100 | 290 | 80 |
| 122 | 16 | TBD | 19 | 100 | | | TBD | 89 |
| 116 | 16 | TBD | 3 | 100 | 10 | 100 | 10 | 100 |
| 107 | 14 | 7.3 | 400 | 100 | 127 | 100 | 660 | 45 |
| 109 | 14 | TBD | TBD | TBD | | | 681 | 80 |
| 106 | 13 | 2.5 | 102 | 100 | 33 | 100 | 380 | 87 |
| 102 | 13 | 8.5 | 127 | 92 | 127 | 100 | 650 | 62 |
| 154 | 13 | TBD | 16 | 100 | | | 150 | 100 |
| 155 | 13 | TBD | 56 | 100 | 60 | 100 | 170 | 100 |
| 101 | 13 | TBD | N/R | 24 | N/R | 20 | N/R | 12 |
| 148 | 12 | TBD | N/R | 17 | N/R | 28 | TBD | 100 |
| 121 | 11 | TBD | 256 | 99 | | | | |
| 139 | 9 | 5.8 | TBD | TBD | 256 | 100 | | |
| 127 | 9 | TBD | 134 | 100 | 180 | 100 | TBD | 94 |
| 129 | 9 | TBD | 45 | 81 | | | 550 | 86 |
| 103 | 8 | TBD | 157 | 100 | | | | |
| 119 | 7 | TBD | N/R | 33 | N/R | 22 | N/R | 3 |
| 133 | 6 | TBD | N/R | 40 | N/R | 5 | N/R | 0 |
| 150 | 6 | TBD | | | 150 | 100 | | |
| 140 | 5 | TBD | N/R | 15 | N/R | 0 | N/R | 33 |
| 110 | 4 | TBD | 38 | 100 | 80 | 100 | | |
| 112 | 4 | TBD | 300 | 100 | 20 | 100 | | |
| 111 | 4 | TBD | 51 | 100 | 24 | 100 | | |
| 152 | 4 | TBD | | | | | | |
| 151 | 5 | TBD | N/A | N/A | N/A | N/A | N/A | N/A |
| 186 | 16 | 15.5 | 200 | 100 | | | | |
| 173 | 14 | 3.8 | 463 | 100 | | | | |
| 174 | 15 | 20.2 | 44 | 100 | 3.8 | 100 | | |
| 175 | 15 | 13.3 | 7 | 100 | 3.8 | 100 | | |
| 219 | 3 | TBD | 48 | 100 | | | | |
| 185 | 10 | 221.7 | 30 | 100 | | | | |
| 184 | 5 | 65.7 | 36 | 100 | | | | |
| 168 | 15 | 16.8 | 22 | 100 | | | | |
| 218 | 8 | TBD | 14 | 100 | | | | |
| 183 | 14 | 25.9 | 17 | 100 | | | | |
| 182 | 14 | TBD | 72 | 100 | | | | |
| 181 | 14 | 22.5 | 23 | 100 | | | | |
| 180 | 12 | TBD | 13 | 100 | | | | |
| 179 | 9 | TBD | 22 | 100 | | | | |
| 178 | 1 | TBD | 39 | 100 | | | | |
| 177 | 6 | TBD | 27 | 100 | | | | |
| 176 | 2 | TBD | 85 | 100 | | | | |

N/A: not active
N/R: not relevant
TBD: To be determined

The in vitro results confirm the target, as determined by the thermal shift caused by the binding of the herbicidal compounds to it. Generally speaking, these compounds show strong herbicidal activity on dicot plants when administered in pre- and/or post-emergence fashion. Also, pre-emergence treatment of monocot plants resulted in strong growth inhibition by most compounds.

The in-planta LTP (Low throughput) results for selected compounds are presented in Table 7 and Table 8 below:

TABLE 7
Herbicidal activity, by phenotype, (in planta, LTP - pre-emergence) for compounds
according to this invention (in parenthesis appears the % effect).

| | Monocot (M)/ | | | | | | |
|---|---|---|---|---|---|---|---|
| Plant | Dicot (D) | 116 | 106 | 110 | 102 | 108 | 129 |
| SETVI | M | DE⁶ (100) | IG¹ (100), | IG (80) | IG (100), | IG (100), | IG (40) |
| | | | DE (100) | | DE (100) | DE (100) | |
| ALOMY | M | IG (100), | | | | | |
| | | W⁵ (100) | | | | | |
| LOLPE | M | DE (100) | DE (60) | | | | IG (40) |
| POAAN | M | DE (100) | IG (100), | | IG (60) | IG (80), | IG (80) |
| | | | DE (100) | | | DE (80) | DE (80) |
| ECHCO | M | DE (100) | | | | | |
| DIGSA | M | DE (100) | | | | | |
| SOLNI | D | IG (80), | | | | | |
| | | DAB² (80) | | | | | |
| EPPHL | D | IG (60), | | | | | |
| | | BL (80), | | | | | |
| | | DAB (80) | | | | | |
| AMAPA | D | IG (80), | DAB (60), | | IG (60) | IG (60) | IG (40) |
| | | C³ (60), | IG (60) | | | | |
| | | DAB (100) | | | | | |
| MATCA | D | IG (80), | | | | | |
| | | BL⁴ (40), | | | | | |
| | | DAB (60) | | | | | |
| ABUTH | D | IG (80), | | | | | |
| | | Bl (80), | | | | | |
| | | DAB (80) | | | | | |
| CHEAL | D | IG (80), | | | | | |
| | | DAB (80) | | | | | |

| | Monocot (M)/ | | | | | |
|---|---|---|---|---|---|---|
| Plant | Dicot (D) | 154 | 109 | 148 | 111 | 112 |
| SETVI | M | DE (100) | DE (100) | DRD⁷ (100), | DE (100), | DE (80), |
| | | | | DE (100), | IG (100) | IG (100) |
| | | | | IG (100) | | |
| ALOMY | M | DE (40), | | DRD (60) | IG (40) | |
| | | IG (80), | | | | |
| | | DAB (60), | | | | |
| | | BL (60) | | | | |
| LOLPE | M | IG (60) | | | | IG (40) |
| POAAN | M | DE (100) | DE (100), | IG (40), | DE (100), | DE (100), |
| | | | IG (100) | DRD (40) | IG (100) | IG (100) |
| ECHCO | M | | | | | |
| DIGSA | M | | | | | |
| SOLNI | D | | | | | |
| EPPHL | D | | | | | |
| AMAPA | D | IG (60) | IG (60) | | IG (60) | IG (60) |
| MATCA | D | | | DE (40) | | IG (60) |

¹IG Inhibited growth
²DAB Disturbed apical bud
³C Chlorosis
⁴BL Bleaching
⁵W Wilting
⁶DE Disturbed emergence
⁷DRD Disturbed root development

TABLE 8
Herbicidal activity, by phenotype, (in planta, LTP-post emergence) for compound
according to this invention (in parenthesis appears the % effect).

| | Monocot (M)/ | | | | | | |
|---|---|---|---|---|---|---|---|
| Plant | Dicot (D) | 106 | 129 | 154 | 109 | 111 | 112 |
| SETVI | M | | | IG (60) | | IG (60) | B (40) |
| POAAN | M | | | IG (60) | | | |
| ECHCO | M | IG¹ (60) | | | | | |
| AMAPA | D | BL² (60) | | BL (60) | BL (40) | BL (40) | IG (60), |
| | | | | | | | B (40) |
| MATCA | D | | LOD³ (40) | | | | |
| ABUTH | D | BL (60) | | | | BL (40) | |

¹IG Inhibited growth
²BL Bleaching
³LOD Lodging

The results show that these compounds can effectively control the growth of dicot and monocot weeds when applied in a pre- or post-emergence fashion. Treated weeds mostly displayed disturbed emergence, inhibited growth, and disturbed apical bud.

1-55. (canceled)

56. A compound represented by the structure of formula I(d): 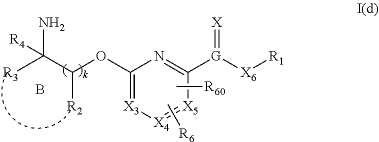
wherein
B ring is absent, or is a substituted or unsubstituted heteroaromatic ring system, or a substituted or unsubstituted C₃-C₁₀ cycloalkyl, or a substituted or unsubstituted C₃-C₁₀ heterocyclic ring (e.g., cyclopropyl, cyclopentyl, cyclohexyl, pyridyl, pyrrolidinyl);
R₁ is H, substituted or unsubstituted C₁-C₁₀ linear or branched, or C₃-C₈ cyclic alkyl (e.g., methyl, ethyl, propyl, butyl, CH₂—CCH, CH₂—C(O)OH, CH₂CH₂C(O)OH), C(O)—R₁₀ (e.g., C(O)—CH₃), R₈—OH (e.g., CH₂—OH), R₈—C(O)O—R₁₀ (e.g., CH₂—C(O)OH, CH₂—C(O)O—CH₃, CH₂—C(O)O—CH₂CH₃, CH₂—CH₂—C(O)OH, CH₂—CH₂—C(O)O—CH₃, CH₂—CH₂—C(O)O—CH₂CH₃), R₈—SH, —R₈—O—R₁₀, (e.g., —CH₂—O—CH₃), R₈—(C₃-C₈ cycloalkyl), R₈-(3-8 membered heterocyclic ring), CF₃, NH₂, NHR, N(R)₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted C₃-C₈ heterocyclic ring, substituted or unsubstituted aryl (e.g., phenyl), or substituted or unsubstituted benzyl
R₂ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(Rn), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₃ is H, substituted or unsubstituted C₁-C₁₀ linear or branched, or C₃-C₈ cyclic alkyl (e.g., methyl, ethyl, propyl, CH₂—CCH, C(H)(OH)(CH₃)), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(Rn) (e.g. CH₂—NH₂); C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₂ and R₃ are joined to form ring B as described above;
R₄ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(Rn) (e.g. CH₂—NH₂), C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
or R₃ and R₄ are joined to form a C₃-C₈ substituted or unsubstituted, saturated or unsaturated, aliphatic or aromatic, carbocyclic or heterocyclic ring (e.g. cyclopropyl), C═O or ═N—OH;
R₆ and R₆₀ are each independently H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(Rn), R₉—R₈—N(R₁₀)(Rn), B(OH)₂, —OC(O)CF₃, —OCH₂Ph, NHC(O)—R₁₀ (e.g., NHC(O)CH₃), NHC(O)—N(R₁₀)(Rn) (e.g., NHC(O)N(CH₃)₂), C(O)OH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₅ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₅ linear or branched, substituted or unsubstituted alkenyl (e.g., ethenyl), C₂-C₅ linear or branched, substituted or unsubstituted alkynyl (e.g., ethynyl, CC—CH₂-cyclobutyl, CC—CF₃), C₁-C₅ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₅ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₅ linear or branched thioalkoxy, C₁-C₅ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl,
X₃, X₄, X₅ are each independently C or N;
X₆ is O or CH₂;
G=X is C═O or SO₂ or G(═X)—X₆—R₁ is a tetrazole moiety;
R₈ is [CH₂]_{q}
wherein p is between 1 and 10;
R₉ is [CH]_{q}, [C]_{q}
wherein q is between 2 and 10;
R₁₀ and R₁₁ are each independently H, CN, C₁-C₅ linear or branched alkyl (e.g., methyl, ethyl), C(O)R (e.g., C(O)(OCH₃)), or S(O)₂R;
or R₁₀ and R₁₁ are joined to form a substituted or unsubstituted 3-8 membered heterocyclic ring;
R is H, C₁-C₅ linear or branched alkyl (e.g., methyl, ethyl), C₁-C₅ linear or branched alkoxy (e.g., methoxy), phenyl, aryl or heteroaryl, or two gem R substituents are joined together to form a 5 or 6 membered heterocyclic ring;
k is an integer number between 1 and 3 (e.g., 1, 2);
or a compound represented by the structure of formula I(e): 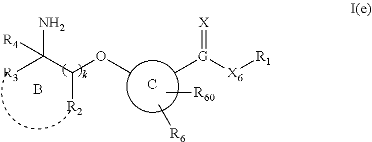
wherein
B ring is absent, or is a substituted or unsubstituted single or fused aromatic or heteroaromatic ring system, or a substituted or unsubstituted single or fused C₃-C₁₀ cycloalkyl, or a substituted or unsubstituted single or fused C₃-C₁₀ heterocyclic ring;
C ring is a saturated or unsaturated, 5 membered heterocyclic ring (e.g., isoxazole, triazole, pyrazole, oxadiazole, furan, thiazole, pyrrole, thiophene), a cyclohexane or a cyclohexene ring;
R₁ is H, substituted or unsubstituted C₁-C₁₀ linear or branched, or C₃-C₈ cyclic alkyl (e.g., methyl, ethyl, propyl, butyl, CH₂—CCH, CH₂—C(O)OH, CH₂CH₂C(O)OH), C(O)—R₁₀ (e.g., C(O)—CH₃), R₈—OH (e.g., CH₂—OH), R₈—C(O)O—R₁₀ (e.g., CH₂—C(O)OH, CH₂—C(O)O—CH₃, CH₂—C(O)O—CH₂CH₃, CH₂—CH₂—C(O)OH, CH₂—CH₂—C(O)O—CH₃, CH₂—CH₂—C(O)O—CH₂CH₃), R₈—SH, —R₈—O—R₁₀, (e.g., —CH₂—O—CH₃), R₈—(C₃-C₈ cycloalkyl), R₈-(3-8 membered heterocyclic ring), CF₃, NH₂, NHR, N(R)₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(Rn), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
R₂ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(Rn), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₃ is H, substituted or unsubstituted C₁-C₁₀ linear or branched, or C₃-C₈ cyclic alkyl (e.g., methyl, ethyl, propyl, CH₂—CCH, C(H)(OH)(CH₃)), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(Rn) (e.g. CH₂—NH₂); C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₂ and R₃ are Joined to form ring B as described above;
R₄ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(R₁₁) (e.g. CH₂—NH₂), C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
or R₃ and R₄ are joined to form a C₃-C₈ substituted or unsubstituted, saturated or unsaturated, aliphatic or aromatic, carbocyclic or heterocyclic ring (e.g. cyclopropyl), C═O or ═N—OH;
R₆ and R₆₀ are each independently H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), COOH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₅ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₅ linear or branched, substituted or unsubstituted alkenyl (e.g., ethenyl), C₂-C₅ linear or branched, substituted or unsubstituted alkynyl (e.g., ethynyl, CC—CH₂-cyclobutyl, CC—CF₃), C₁-C₅ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₅ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₅ linear or branched thioalkoxy, C₁-C₅ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl,
X₆ is O, NH, NR or CH₂;
G=X is C═O or SO₂ or G(═X)—X₆—R₁ is a tetrazole moiety;
R₈ is [CH₂]_{q}
wherein p is between 1 and 10;
R₉ is [CH]_{q}, [C]_{q}
wherein q is between 2 and 10;
R₁₀ and R₁₁ are each independently H, CN, C₁-C₅ linear or branched alkyl (e.g., methyl, ethyl), C(O)R (e.g., C(O)(OCH₃)), or S(O)₂R;
or R₁₀ and R₁₁ are joined to form a substituted or unsubstituted 3-8 membered heterocyclic ring;
R is H, C₁-C₅ linear or branched alkyl (e.g., methyl, ethyl), C₁-C₅ linear or branched alkoxy (e.g., methoxy), phenyl, aryl or heteroaryl, or two gem R substituents are joined together to form a 5 or 6 membered heterocyclic ring;
k is an integer number between 1 and 3 (e.g., 1, 2);
or a compound represented by any one of the following structures:

| Compound | |
|---|---|
| Number | Structure |
| 161 | 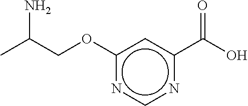 |
| 178 | 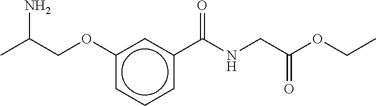 |
| 186 | 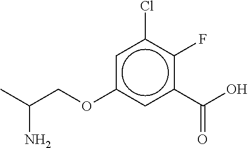 |
| 187 | 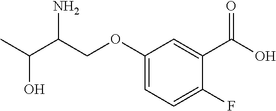 [SMILES: CC(O)C(N)COc1ccc(F)c(C(=O)O)c1] |
| 189 | 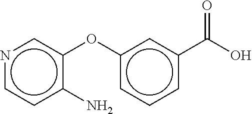 |
| 214 | 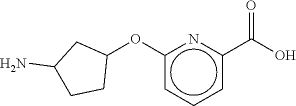 |

or its agrochemically acceptable salt, zwitterion (inner salt), hydrate, N-oxide, isotopic variant or any combination thereof,
wherein the compound is not: 6-(2-aminopropoxy)picolinic; 6-(2-aminopropoxy)pyrazine-2-carboxylic acid, 2-(2-aminopropoxy)pyrimidine-4-carboxylic acid; 6-(2-aminopropoxy)-3-fluoropicolinic acid; 6-(2-aminopropoxy)-4-fluoropicolinic acid; 6-(2-aminobutoxy)picolinic acid; 6-((2-aminocyclopentyl)oxy)picolinic acid; methyl 6-(2-aminopropoxy)picolinate; or ethyl 6-(2-aminopropoxy)picolinate.

57. The compound according to claim 56 as defined by formula I(d),
wherein said “substituted” is substituted with at least one substitution selected from: F, Cl, Br, I, C₁-C₅ linear or branched alkyl, C₂-C₅ linear or branched alkenyl (e.g., ethenyl), C₂-C₅ linear or branched alkynyl, alkyl amide (i.e., C(O)NH—R or NHC(O)—R), C(O)OH, alkyl ester (i.e., C(O)OR or OC(O)—R), OH, alkoxy, N(R)₂, NH₂, CF₃, aryl, phenyl, heteroaryl, C₃-C₈ cycloalkyl, R₈—(C₃-C₈ cycloalkyl), halophenyl, CN, and NO₂;
wherein at least one of R₆ and R₆₀ is not H or F;
wherein X₅ is N;
wherein R₁ is not H, methyl or ethyl;
wherein R₂ and R₃ are not joined to form a cyclopentyl ring;
wherein R₃ and R₄ are both H or are both methyl;
wherein k is not 1;
wherein at least one of R₆ and R₆₀ is not H or F;
wherein at least one of R₆ and R₆₀ is alkyl, Cl, CF₃, CN, substituted or unsubstituted alkoxy, substituted or unsubstituted alkyne, or heteroaromatic ring;
wherein R₁ is not H, methyl or ethyl;
wherein R₁ is R₈—C(O)O—R₁₀, CH₂CH₂—C(O)O—CH₂CH₃, CH₂—C(O)O—CH₂CH₃, CH₂—C(O)O—CH₃, CH₂—C(O)OH, CH₂CH₂—C(O)OH, CH₂CH₂—C(O)O—CH₃, C₃-C₈ alkyl, butyl, or CH₂—CCH;
wherein the compound is a substantially pure single stereoisomer;
wherein the compound comprises a single enantiomer in a purity of >80%;
wherein R₃ and R₄ are both H or both methyl, and R₂ is a H or methyl;
or any combination thereof.

58. The compound of claim 57, wherein the substantial pure single stereoisomer has a purity higher than 90%, preferably higher than 95%, most preferably higher than 98%.

59. The compound according to claim 56 as defined by formula I(d), represented by any one of the following structures:

| Compound | |
|---|---|
| Number | Structure |
| 157 | 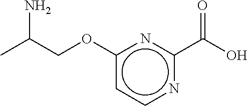 |
| 174 | 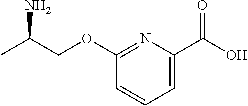 |
| 175 | 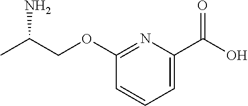 |
| 176 | 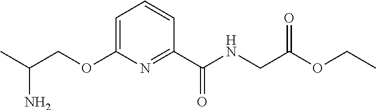 [SMILES: CCOC(=O)CNC(=O)c1cccc(OCC(C)N)n1] |
| 177 | 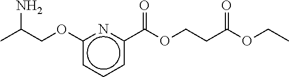 |
| 179 | 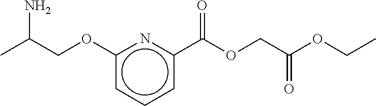 |
| 180 | 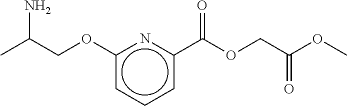 |
| 181 | 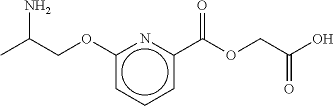 |
| 182 | 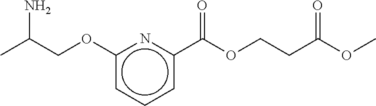 |
| 183 | 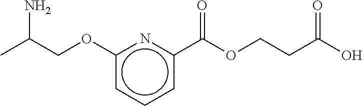 |
| 184 | 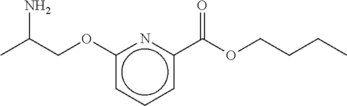 |
| 185 | 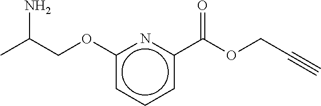 |
| 188 | 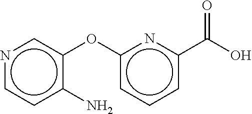 |
| 190 | 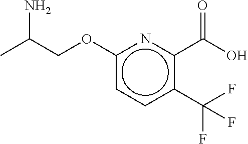 |
| 191 | 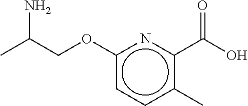 |
| 192 | 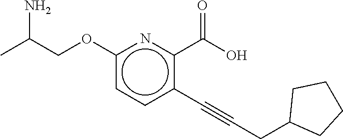 |
| 193 | 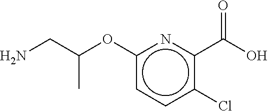 |
| 194 | 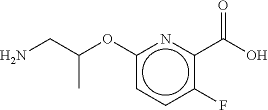 |
| 195 | 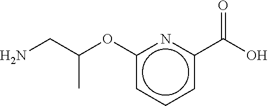 |
| 196 | 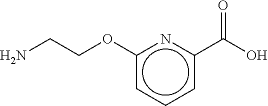 |
| 197 | 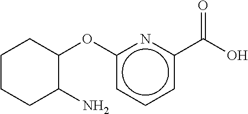 |
| 198 | 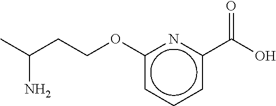 |
| 199 | 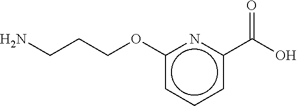 |
| 200 | 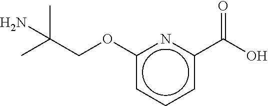 |
| 201 | 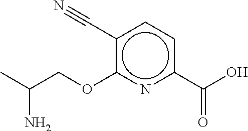 |
| 202 | 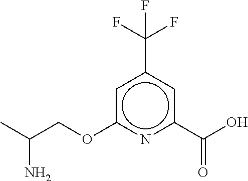 |
| 203 | 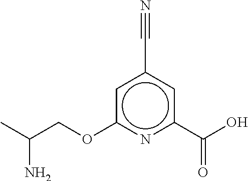 |
| 204 | 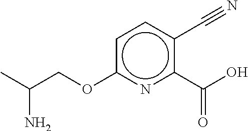 |
| 205 | 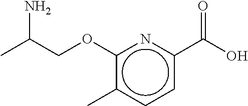 |
| 206 | 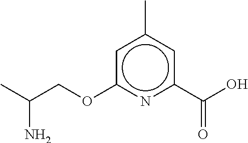 |
| 207 | 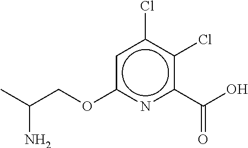 |
| 208 | 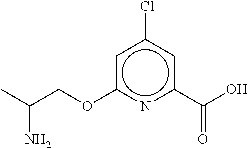 |
| 209 | 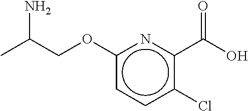 |
| 210 | 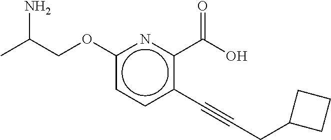 |
| 211 | 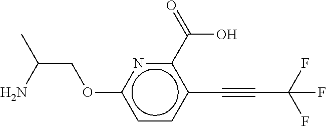 |
| 212 | 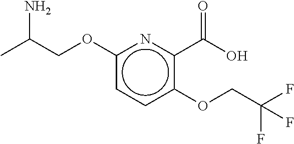 |
| 213 | 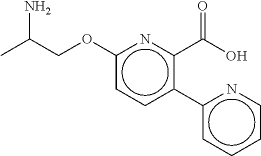 |
| 215 | 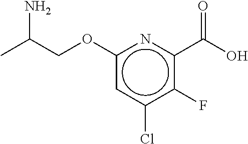 |
| 216 | 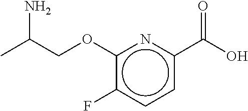 |

60. The compound of claim 56, as defined by formula I(e), selected from any one of the following structures:

| Compound | |
|---|---|
| Number | Structure |
| 169 | 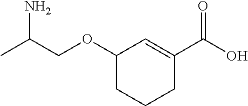 |
| 170 | 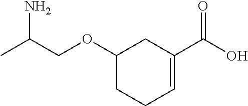 |
| 171 | 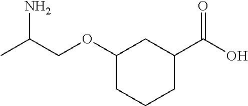 |
| 400 | 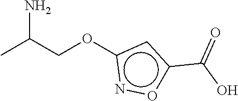 |
| 401 | 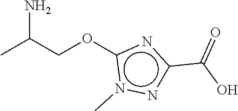 |
| 402 | 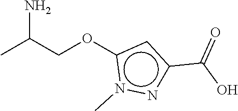 |
| 403 | 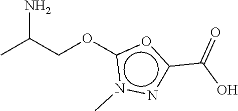 |
| 404 | 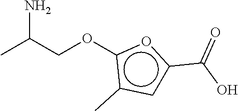 |
| 405 | 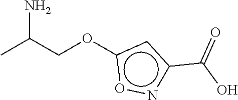 |
| 406 | 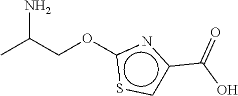 |
| 407 | 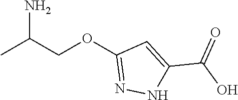 |
| 408 | 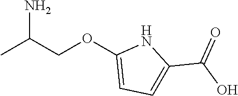 |
| 409 | 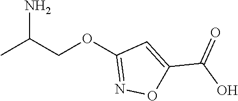 |
| 410 | 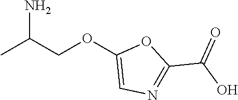 |
| 411 | 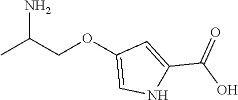 |
| 412 | 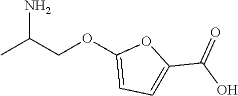 |
| 413 | 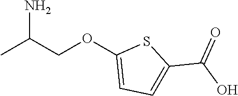 |
| 414 | 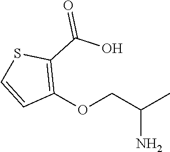 |
| 415 | 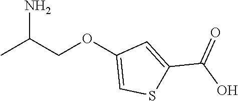 |

61. The compound of claim 56, wherein the compound is an herbicide or a pesticide.

62. An agrochemical composition comprising a pesticidally and/or herbicidally effective amount of a compound as defined in claim 56, and an agrochemically-acceptable diluent or carrier.

63. A method of controlling the growth of undesired plants, comprising applying a compound according to claim 56, to crop fields.

64. The method of claim 63,
wherein the plant is a eudicot (dicot) or a monocotyledon (monocot);
wherein the plant is a weed;
or combination thereof.

65. The method of claim 64,
wherein said weed comprises: Abutilon theophrasti, Amaranthus palmeri, Ambrosia artemisiifolia, Alopecurus myosuroides, Avena sterilis, Chenopodium album, Conyza Canadensis, Digitaria sanguinalis, Echinochloa colona, Euphorbia heterophylla, Lolium perenne, Lolium rigidum, Matricaria chamomilla, Phalaris paradoxa, Poa annua, Portulaca oleracea, Setaria viridis, Solanum nigrum or any combination thereof;
wherein the dicot plant is Arabidopsis thaliana, and/or the monocot plant is Dactyloctenium aegyptium or Eragrostis teff;
or combination thereof.

66. The method of claim 63, for use in pre-plant treatments, pre-emergence treatments, post-emergence treatments, or any combination thereof.

67. A method for controlling the growth of undesired plants, comprising applying a compound or an agrochemical composition thereof, to a crop field, wherein the compound is represented by the structure of formula I: 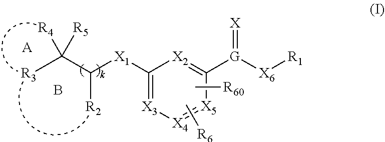
wherein
A ring is absent, or is a substituted or unsubstituted single or fused aromatic or heteroaromatic ring system, or a substituted or unsubstituted single or fused C₃-C₁₀ cycloalkyl, or a substituted or unsubstituted single or fused C₃-C₁₀ heterocyclic ring (e.g., cyclopropyl);
B ring is absent, or is a substituted or unsubstituted single or fused heteroaromatic ring system, or a substituted or unsubstituted single or fused C₃-C₁₀ cycloalkyl, or a substituted or unsubstituted single or fused C₃-C₁₀ heterocyclic ring (e.g., cyclopentyl);
R₁ is H, substituted or unsubstituted C₁-C₁₀ linear or branched, or C₃-C₈ cyclic alkyl (e.g., methyl, ethyl, propyl, butyl, CH₂—CCH, CH₂—C(O)OH, CH₂CH₂C(O)OH), C(O)—R₁₀ (e.g., C(O)—CH₃), R₈—OH (e.g., CH₂—OH), R₈—C(O)O—R₁₀ (e.g., CH₂—C(O)OH, CH₂—C(O)O—CH₃, CH₂—C(O)O—CH₂CH₃, CH₂—CH₂—C(O)OH, CH₂—CH₂—C(O)O—CH₃, CH₂—CH₂—C(O)O—CH₂CH₃), R₈—SH, —R₈—O—R₁₀, (e.g., —CH₂—O—CH₃), R₈—(C₃-C₈ cycloalkyl), R₈-(3-8 membered heterocyclic ring), CF₃, NH₂, NHR, N(R)₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(Rn), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
R₂ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, R₈—N(R₁₀)(R₁₁), C(O)H, C₁-C₅ linear or branched C(O)-haloalkyl (e.g., C(O)—CF₃), —C(O)NH₂, C(O)NHR, C(O)N(R₁₀)(Rn), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
R₃ is H, substituted or unsubstituted C₁-C₁₀ linear or branched, or C₃-C₈ cyclic alkyl (e.g., methyl, ethyl, propyl, CH₂—CCH, C(H)(OH)(CH₃)), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(Rn) (e.g. CH₂—NH₂); C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl;
or R₂ and R₃ are joined to form a C₃-C₈ substituted or unsubstituted, saturated or unsaturated, aliphatic (e.g. cyclopentyl, cyclohexyl), or aromatic (e.g. pyridine), carbocyclic or heterocyclic ring (e.g. pyrrolidine);
R₄ is H, substituted or unsubstituted C₁-C₁₀ linear or branched, or C₃-C₈ cyclic alkyl (e.g., methyl, ethyl, propyl, CH₂—CCH), F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CN, NO₂, NH₂, NHR, N(R)₂, R₈—N(R₁₀)(Rn) (e.g. CH₂—NH₂), C(O)H, C₁-C₅ linear or branched C(O)-haloalkyl (e.g., C(O)—CF₃), —C(O)NH₂, C(O)NHR, C(O)N(R₁₀)(R₁₁), C₂-C₅ linear or branched, substituted or unsubstituted alkenyl (e.g., CH═C(Ph)₂), C₁-C₅ linear or branched or C₃-C₈ cyclic haloalkyl, substituted or unsubstituted 3-8 membered heterocyclic ring, substituted or unsubstituted aryl (e.g., phenyl), or substituted or unsubstituted benzyl
or R₃ and R₄ are joined to form a C₃-C₈ substituted or unsubstituted, saturated or unsaturated, aliphatic or aromatic, carbocyclic or heterocyclic ring (e.g. cyclopropyl), C═O or ═N—OH;
R₅ is H, OH, SH, NH₂, NHNH₂, NHR, N(R₁₀)(R₁₁) (e.g., N(H)CH₃, N(H)CH₂CH₃), R₈—N(R₁₀)(R₁₁) (e.g. CH₂—NH₂); or R₂ and R₅ are joined to form a substituted or unsubstituted 4-8 membered heterocyclic ring (e.g. pyrrolidine);
R₆ and R₆₀ are each independently H, F, Cl, Br, I, OH, SH, R₈—OH (e.g., CH₂—OH), R₈—SH, —R₈—O—R₁₀, (e.g., —CH₂—O—CH₃), R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀ (e.g., NHC(O)CH₃), NHC(O)—N(R₁₀)(R₁₁) (e.g., NHC(O)N(CH₃)₂), C(O)OH, —C(O)Ph, C(O)O—R₁₀ (e.g. C(O)O—CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR, C(O)N(R₁₀)(R₁₁) (e.g., C(O)N(CH₃)₂), SO₂R, SO₂N(R₁₀)(R₁₁) (e.g., SO₂N(CH₃)₂, SO₂NHC(O)CH₃), C₁-C₅ linear or branched, substituted or unsubstituted alkyl (e.g., methyl, ethyl, propyl, iso-propyl, t-Bu, iso-butyl, pentyl, benzyl), substituted or unsubstituted C₃-C₈ cycloalkyl (e.g., cyclopropyl, cyclopentyl), C₂-C₅ linear or branched, substituted or unsubstituted alkenyl (e.g., ethenyl), C₂-C₅ linear or branched, substituted or unsubstituted alkynyl (e.g., ethynyl, CC—CH₂-cyclobutyl, CC—CF₃), C₁-C₅ linear or branched or C₃-C₈ cyclic haloalkyl (e.g., CF₃, CF₂CH₃, CH₂CF₃, CF₂CH₂CH₃, CH₂CH₂CF₃, CF₂CH(CH₃)₂, CF(CH₃)—CH(CH₃)₂), substituted or unsubstituted C₁-C₅ linear or branched or C₃-C₈ cyclic alkoxy (e.g. methoxy, ethoxy, propoxy, isopropoxy, O—CH₂—CF₃), optionally wherein at least one methylene group (CH₂) in the alkoxy is replaced with an oxygen atom, C₁-C₅ linear or branched thioalkoxy, C₁-C₅ linear or branched haloalkoxy (e.g., OCF₃, OCHF₂), substituted or unsubstituted 3-8 membered heterocyclic ring (e.g., thiophene, oxazole, oxadiazole, imidazole, furane, triazole, tetrazole, pyridine, pyrimidine, pyrazine), substituted or unsubstituted aryl (e.g., phenyl), substituted or unsubstituted benzyl,
X₁ is O, S, NH, NR (e.g., N—CH₃), S═O, SO₂; or X₁ and R₄ are joined to form a C₄-C₈ heterocyclic ring (e.g., pyrrolidine);
X₂, X₃, X₄, X₅ are each independently C or N;
X₆ is O, NH, NR or CH₂;
G=X is C═O or SO₂ or G(═X)—X₆—R₁ is a tetrazole moiety;
R₈ is [CH₂]_{q}
wherein p is between 1 and 10;
R₉ is [CH]_{q}, [C]_{q}
wherein q is between 2 and 10;
R₁₀ and R₁₁ are each independently H, CN, C₁-C₅ linear or branched alkyl (e.g., methyl, ethyl), C(O)R (e.g., C(O)(OCH₃)), or S(O)₂R;
or R₁₀ and R₁₁ are joined to form a substituted or unsubstituted 3-8 membered heterocyclic ring;
R is H, C₁-C₅ linear or branched alkyl (e.g., methyl, ethyl), C₁-C₅ linear or branched alkoxy (e.g., methoxy), phenyl, aryl or heteroaryl, or two gem R substituents are joined together to form a 5 or 6 membered heterocyclic ring;
k is an integer number between 1 and 3 (e.g., 1, 2);
or its agrochemically acceptable salt, zwitterion (inner salt), stereoisomer, tautomer, hydrate, N-oxide, reverse amide analog, isotopic variant (e.g., deuterated analog), or any combination thereof.

68. The method of claim 67,
wherein ring A is absent or is a cyclopropyl, or ring B is absent or is a cyclopentyl or cyclohexyl;
wherein R₁ is H, substituted or unsubstituted C₁-C₁₀ linear or branched, or C₃-C₈ cyclic alkyl, C(O)—R₁₀, R₈—OH, R₈—C(O)O—R₁₀, or C₂-C₅ linear or branched alkenyl;
or combination thereof.

69. The method of claim 68, wherein the alkyl is methyl, ethyl, propyl, butyl, CH₂—CCH, CH₂—C(O)OH or CH₂CH₂C(O)OH; the C(O)—R₁₀ is C(O)—CH₃; the alkenyl is ethenyl; the R₈—OH is CH₂—OH; and/or the R₈—C(O)O—R₁₀ is CH₂—C(O)OH, CH₂—C(O)O—CH₃, CH₂—C(O)O—CH₂CH₃, CH₂—CH₂—C(O)OH, CH₂—CH₂—C(O)O—CH₃ or CH₂—CH₂—C(O)O—CH₂CH₃.

70. The method of claim 67,
wherein k is 1 or 2;
wherein R₂ is H, substituted or unsubstituted C₁-C₁₀ linear or branched, or C₃-C₈ cyclic alkyl (e.g., methyl);
wherein R₃ is H, substituted or unsubstituted C₁-C₁₀ linear or branched, or C₃-C₈ cyclic alkyl, or R₈—N(R₁₀)(Rn);
wherein R₄ is H, substituted or unsubstituted C₁-C₁₀ linear or branched, or C₃-C₈ cyclic alkyl,
or wherein R₂ and R₃ are joined to form a 3-8 membered, substituted or unsubstituted, saturated or unsaturated, aliphatic or aromatic carbocyclic or heterocyclic ring,
or wherein R₃ and R₄ are joined to form a C₄-C₈ substituted or unsubstituted, saturated or unsaturated, aliphatic or aromatic carbocyclic or heterocyclic ring.

71. The method of claim 70,
wherein the alkyl is methyl, ethyl, or C(H)(OH)(CH₃); R₈—N(R₁₀)(R₁₁) is CH₂—NH₂; and the C₄-C₈ substituted or unsubstituted, saturated or unsaturated, aliphatic or aromatic carbocyclic or heterocyclic ring is cyclopropyl, cyclopentyl, cyclohexyl, pyrrolidinyl or pyridyl;
wherein R₃ and R₄ are joined to form a cyclopropyl ring, C═O or a ═N—OH;
wherein R₄ is H, substituted or unsubstituted C₁-C₁₀ linear or branched, or C₃-C₈ cyclic alkyl, preferably methyl, or R₄ is R₈—N(R₁₀)(R₁₁) (e.g., CH₂—NH₂);
wherein R₅ is OH, NH₂, N(R₁₀)(R₁₁) (e.g., N(H)CH₃ or N(H)CH₂CH₃) or R₅ is R₈—N(R₁₀)(R₁₁) (e.g., CH₂—NH₂);
wherein X₁ is O, S, NH, or N—CH₃; or wherein X₁ and R₄ are joined to form a C₄-C₅ heterocyclic ring, preferably pyrrolidine;
wherein X₆ is O, NH or NR;
wherein R₆₀ is H, Cl or F;
wherein at least one of R₆ and R₆₀ is not H;
wherein at least one of X₂-X₅ is N, preferably X₂;
wherein R₁ is not H;
wherein the compound is a substantially pure single stereoisomer; preferably wherein the substantial pure stereoisomer has a purity higher than 90%, more preferably higher than 95%, most preferably higher than 98%;
or any combination thereof.

72. The method of claim 67, wherein R₆ is H, F, Cl, Br, I, CF₃, CN, C₁-C₅ linear or branched, substituted or unsubstituted alkyl, C₂-C₅ linear or branched, substituted or unsubstituted alkenyl, C₂-C₅ linear or branched, substituted or unsubstituted alkynyl, C₁-C₅ linear or branched or C₃-C₈ cyclic haloalkyl, substituted or unsubstituted C₁-C₅ linear or branched or C₃-C₈ cyclic alkoxy, or substituted or unsubstituted 3-8 membered heterocyclic ring.

73. The method of claim 72, wherein the alkyl is methyl or ethyl; wherein the alkenyl is ethenyl, the C₁-C₅ linear or branched or C₃-C₈ cyclic haloalkyl is CF₃, the alkoxy is methoxy or O—CH₂—CF₃; the alkynyl is ethynyl, CC—CH₂-cyclobutyl, or CC—CF₃, wherein the substituted or unsubstituted 3-8 membered heterocyclic ring is pyridine; or any combination thereof.

74. The method of claim 67, wherein the compound is represented by any one of the following structures:

| Compound | |
|---|---|
| Number | Structure |
| 101 | 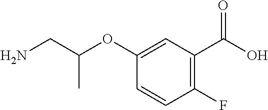 |
| 102 | 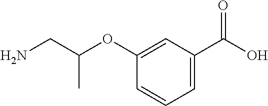 |
| 103 | 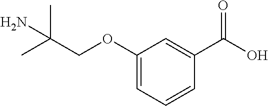 |
| 104 | 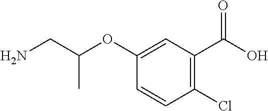 |
| 105 | 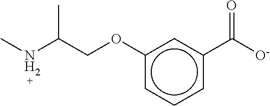 |
| 106 | 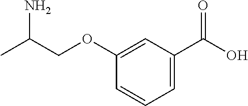 |
| 107 | 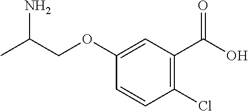 |
| 108 | 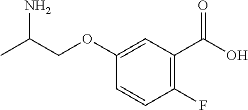 |
| 109 | 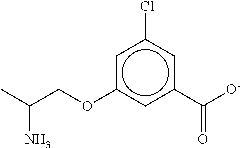 |
| 110 | 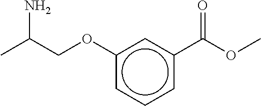 |
| 111 | 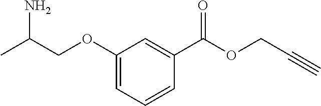 [SMILES: C#CCOC(=O)c1cccc(OCC(C)N)c1] |
| 112 | 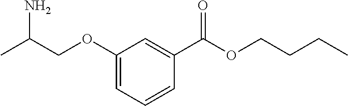 [SMILES: CCCCOC(=O)c1cccc(OCC(C)N)c1] |
| 113 | 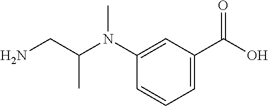 |
| 114 | 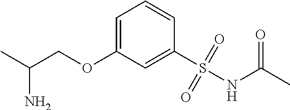 [SMILES: CC(=O)NS(=O)(=O)c1cccc(OCC(C)N)c1] |
| 115 | 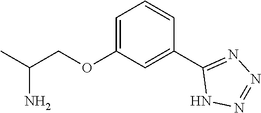 [SMILES: CC(N)COc1cccc(-c2nnn[nH]2)c1] |
| 116 | 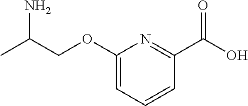 |
| 117 | 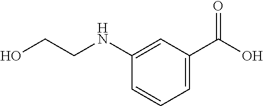 [SMILES: O=C(O)c1cccc(NCCO)c1] |
| 118 | 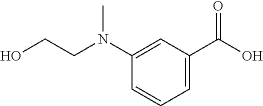 |
| 119 | 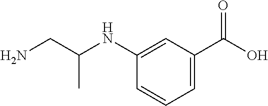 |
| 120 | 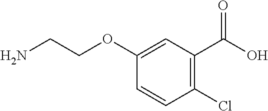 [SMILES: NCCOc1ccc(Cl)c(C(=O)O)c1] |
| 121 | 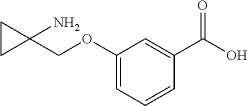 [SMILES: NC1(COc2cccc(C(=O)O)c2)CC1] |
| 122 | 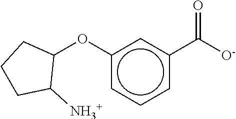 |
| 123 | 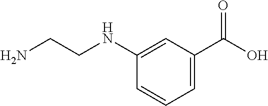 [SMILES: NCCNc1cccc(C(=O)O)c1] |
| 124 | 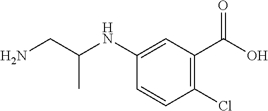 |
| 125 | 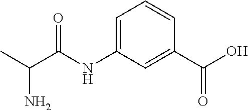 |
| 126 | 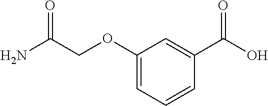 [SMILES: NC(=O)COc1cccc(C(=O)O)c1] |
| 127 | 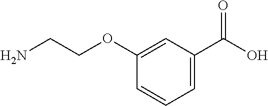 [SMILES: NCCOc1cccc(C(=O)O)c1] |
| 128 | 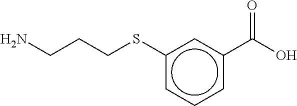 |
| 129 | 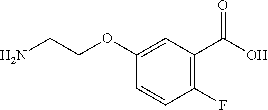 [SMILES: NCCOc1ccc(F)c(C(=O)O)c1] |
| 130 | 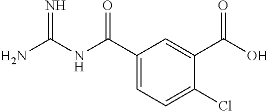 [SMILES: N=C(N)NC(=O)c1ccc(Cl)c(C(=O)O)c1] |
| 131 | 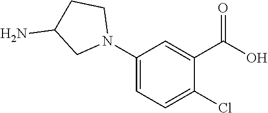 [SMILES: NC1CCN(c2ccc(Cl)c(C(=O)O)c2)C1] |
| 132 | 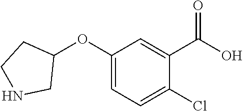 [SMILES: O=C(O)c1cc(OC2CCNC2)ccc1Cl] |
| 133 | 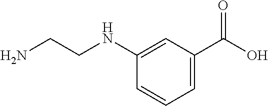 [SMILES: NCCNc1cccc(C(=O)O)c1] |
| 134 | 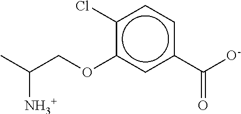 |
| 135 | 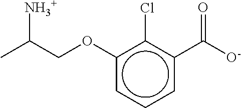 |
| 136 | 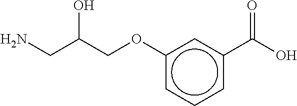 |
| 137 | 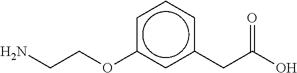 |
| 138 | 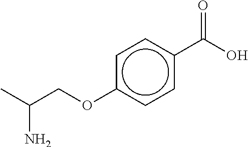 |
| 139 | 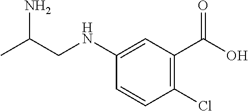 |
| 140 | 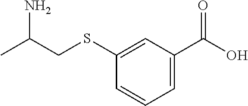 |
| 141 | 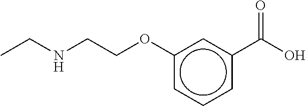 |
| 142 | 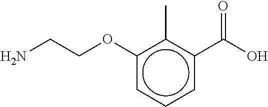 |
| 143 | 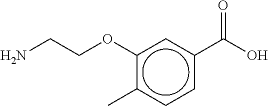 |
| 144 | 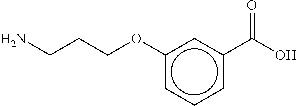 |
| 145 | 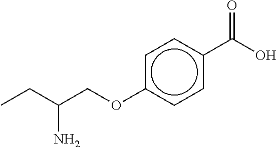 |
| 146 | 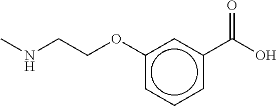 |
| 147 | 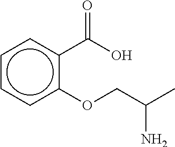 |
| 148 | 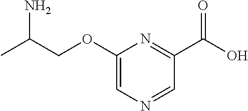 |
| 149 | 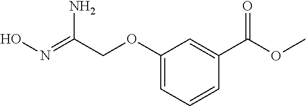 [SMILES: COC(=O)c1cccc(OC/C(N)=N/O)c1] |
| 150 | 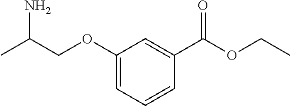 [SMILES: CCOC(=O)c1cccc(OCC(C)N)c1] |
| 151 | 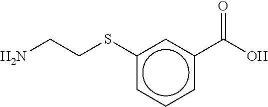 |
| 152 | 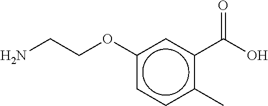 |
| 153 | 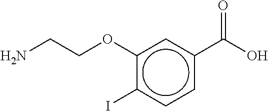 |
| 154 | 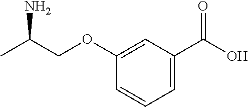 |
| 155 | 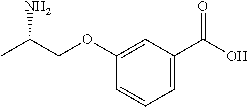 |
| 156 | 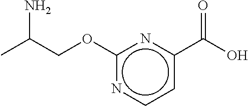 |
| 157 | 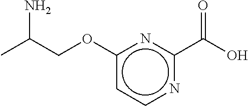 |
| 158 | 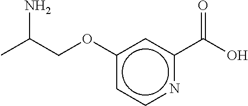 |
| 159 | 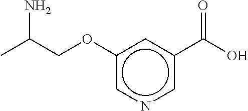 |
| 160 | 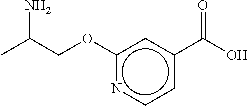 |
| 161 | 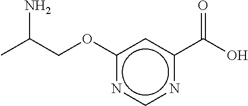 |
| 162 | 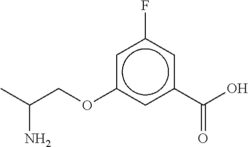 |
| 163 | 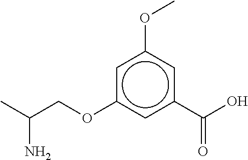 |
| 164 | 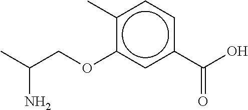 |
| 165 | 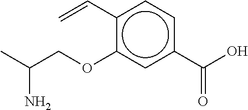 |
| 166 | 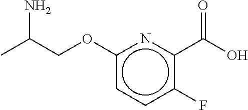 |
| 167 | 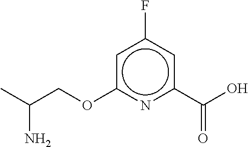 |
| 168 | 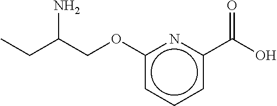 |
| 172 | 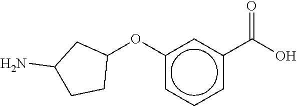 |
| 173 | 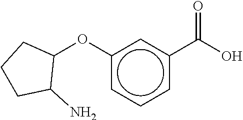 |
| 174 | 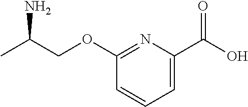 |
| 175 | 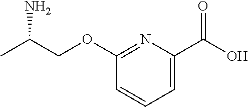 |
| 176 | 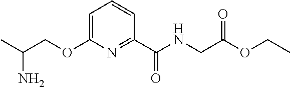 [SMILES: CCOC(=O)CNC(=O)c1cccc(OCC(C)N)n1] |
| 177 | 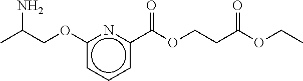 |
| 178 | 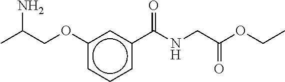 |
| 179 | 402 |
| 180 | 403 |
| 181 | 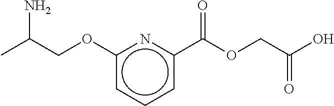 |
| 182 | 405 |
| 183 | 406 |
| 184 | 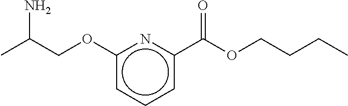 |
| 185 | 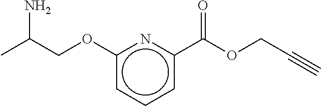 |
| 186 | 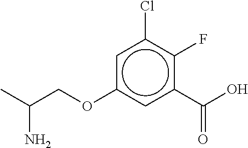 |
| 187 | 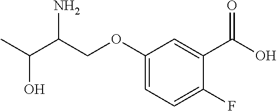 [SMILES: CC(O)C(N)COc1ccc(F)c(C(=O)O)c1] |
| 188 | 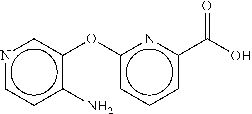 |
| 189 | 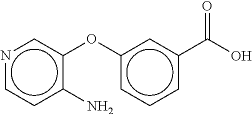 |
| 190 | 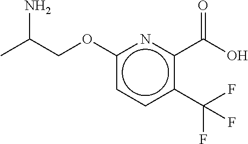 |
| 191 | 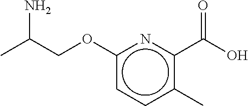 |
| 192 | 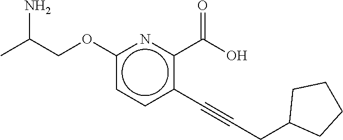 |
| 193 | 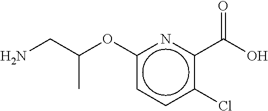 |
| 194 | 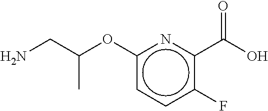 |
| 195 | 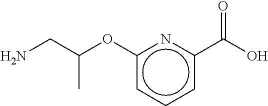 |
| 196 | 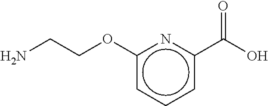 |
| 197 | 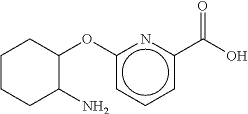 |
| 198 | 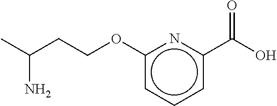 |
| 199 | 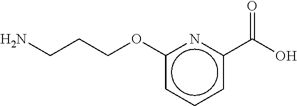 |
| 200 | 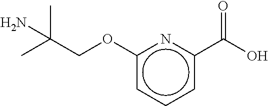 |
| 201 | 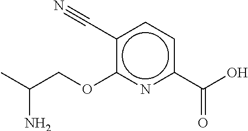 |
| 202 | 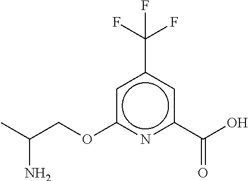 |
| 203 | 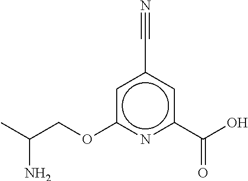 |
| 204 | 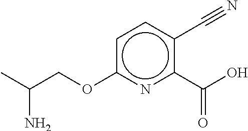 |
| 205 | 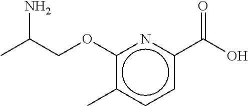 |
| 206 | 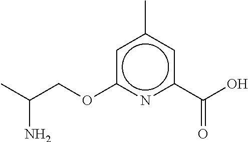 |
| 207 | 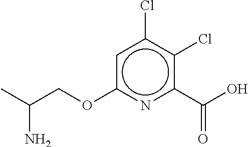 |
| 208 | 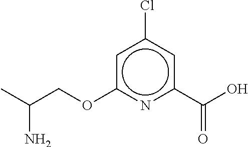 |
| 209 | 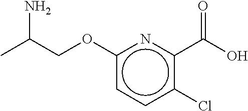 |
| 210 | 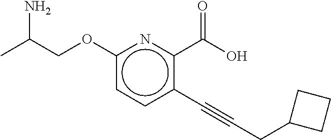 |
| 211 | 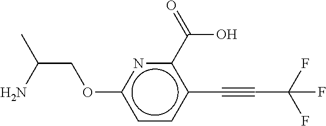 |
| 212 | 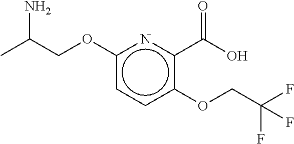 |
| 213 | 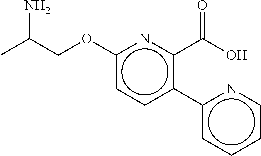 |
| 214 | 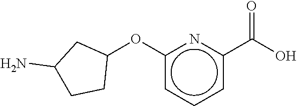 |
| 215 | 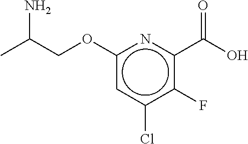 |
| 216 | 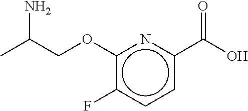 |
| 217 | 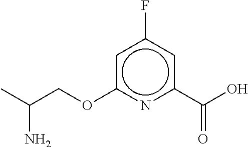 |
| 218 | 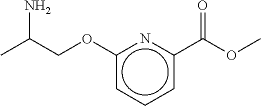 |
| 219 | 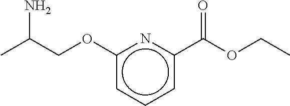 |

75. The method of claim 67,
wherein the compound is an herbicide or a pesticide;
wherein the plant is a eudicot (dicot) or a monocotyledon (monocot);
wherein said plant is a weed;
or combination thereof.

76. The method of claim 75,
wherein said weed comprises: Abutilon theophrasti, Amaranthus palmeri, Ambrosia artemisiifolia, Alopecurus myosuroides, Avena sterilis, Chenopodium album, Conyza Canadensis, Digitaria sanguinalis, Echinochloa colona, Euphorbia heterophylla, Lolium perenne, Lolium rigidum, Matricaria chamomilla, Phalaris paradoxa, Poa annua, Portulaca oleracea, Setaria viridis, Solanum nigrum or any combination thereof;
wherein the dicot plant is Arabidopsis thaliana, and/or the monocot plant is Dactyloctenium aegyptium or Eragrostis teff;
or combination thereof.

77. The method of claim 67, for use in pre-plant treatments, pre-emergence treatments, post-emergence treatments, or any combination thereof.
